(19) United States
(12) Patent Application Publication
(10) Pub. No.: US 2019/0271032 A1
(43) Pub. Date: Sep. 5, 2019

(54) A METHOD FOR AMPLIFICATION OF NUCLEIC ACID SEQUENCES

(71) Applicant: Nucleotrace Pty. Ltd., Toongabbie, Victoria (AU)

(72) Inventor: Nicholas Alexander Owen, Victoria (AU)

(21) Appl. No.: 16/319,546

(22) PCT Filed: Jul. 21, 2017
(86) PCT No.: PCT/AU2017/050757

§ 371 Date: Jan. 22, 2019

(30) Foreign Application Priority Data
Jul. 22, 2016 (AU) 2016902892

(51) Int. Cl. C12Q 1/6848

(52) U.S. Cl. CPC C12Q 1/6848

(57) ABSTRACT

The present invention relates generally to a method for the amplification of nucleic acid sequences, more specifically, to a method for the amplification and identification of target nucleic acid sequences using primers containing locked nucleic acids for tracing a product to its origin.

### TECHNICAL FIELD

The present invention relates generally to a method for the amplification of nucleic acid sequences, more specifically, to a method for the amplification and identification of target nucleic acid sequences.

### BACKGROUND

Counterfeiting and piracy has increased substantially over the last two decades, with counterfeit and pirated products found in almost every country across the globe and in virtually all sectors of the economy. Estimates of the levels of counterfeiting and the value of such products vary. However, the value of global trade in counterfeit and pirated products in 2013 was estimated at $461 billion (OECD and EUIPO, 2016, Trade in Counterfeit and Pirated Goods: Mapping the Economic Impact). Anti-counterfeiting measures are employed by many manufacturers to minimize the impacts of counterfeiting. Such measures include security printing with special watermarks, inks and dyes, holograms, tamper-proof labels, laser surface authentication and magnetic and radio frequency identification (RFID) tags. While all of these methods are somewhat effective, they are generally not counterfeit-proof and may be overcome by forgery. By contrast, molecular tagging of products using nucleic acid molecules, also referred to as molecular “taggants”, has proven to be a virtually counterfeit proof means to authenticate and track products. Illustrative examples include pollutant tracing (e.g. hydrocarbons), product authentication (e.g. artwork, electrical goods), and security applications (e.g. bank note and document authentication).

Nucleic acid molecules are ideal molecular tags (also referred to as “taggants”) because they are inherently stable, information dense, non-toxic, and synthesised and sequenced using commercially mature technologies. Non-biological information may be encoded in fragments of DNA using the nucleic acid base pair (bp) ‘alphabet’, where the set of letters available is S={A (adenine), T (thymine), G (guanine) and C (cytosine)} for DNA and {A (adenine), U (uracil), G (guanine) and C (cytosine)} for RNA, and the size of the set is s=4 (if the letter length l=lbp). This base-four system allows vast amounts of information to be stored in relatively short fragments of DNA, with the number of unique codewords (taggants) available of string length n symbols (‘letters’) being wₙ=sⁿ. Whilst synthetic nucleotide tagging systems are not new, methodologies to efficiently identify and decode a taggant, or a mixed set of taggants, are still lacking. Identification, as distinct from authentication, is where the set of all possible taggants is screened to determine a subset of unknown taggants (Wᵤ⊆Wₙ). Authentication, in contrast, is where the presence of a known subset of taggants is tested (i.e. Wₖ⊆Wₙ) using a particular set or sets of primer ‘keys’. For applications that require identification capabilities, prior knowledge of the primers required to recover individual taggants is absent by definition. Using conventional techniques to identify one object from a pool of millions is clearly not feasible as this would require screening samples with millions of pairs of primer. The capacity to decode a subset of unknown taggants simultaneously additionally offers taggant ‘layering’ capabilities where the elements of a product are marked, combined, and subsequently decoded from the final product.

Existing taggant technologies are reasonably well-suited to authentication applications but are highly inefficient for identification and layering purposes. One way to address this problem of ‘identification vs authentication’ is to design taggant libraries with universal forward and reverse primer site sequences and variable encoding regions that are decoded by fragment length separation or sequencing. However, the use of universal primer site sequences invariably results in cross-fragment hybridization during recovery and amplification steps (usually involving polymerase chain reaction, PCR). This is a particularly difficult problem if taggant codewords are generated from a library of common letter sequences. In this case, the same letter used in two different codewords are likely to form heterodimers in a solution of mixed taggants, resulting in cross-hybridised PCR products. A number of problems precipitate from this fact: (1) each taggant must contain a unique set or sets of distinguishing primer pairs for recovery and amplification, (2) each taggant in a library must use substantially different sequences to encode the same symbols, (3) without prior knowledge of the taggants present in a product (which, by definition, is identification), samples must be screened for all possible primer pairs in the library Wₙ (4) large-scale screenings (e.g. >300 PCR reactions) are impractical, expensive, and not conducive to low fragment copy-number recovery, and (5) these constraints restrict current technologies to a practical taggant library size limit of wₙ<3000 and a layering limit of 20 taggants (U.S. Pat. No. 8,735,327). This has severely restricted the layering capacity, and therefore the potential applications, of existing taggant technologies.

WO 2004/063856 (Connolly, 2004) describes a device for detecting target nucleic acid molecules using an electrical conductor with attached capture probes. The capture probes are complementary to one of the target nucleic acid molecules, which allows for the detection of the nucleic acid molecule when electricity is conducted between the probes. This method is designed to test for the presence of a known target nucleic acid molecule (i.e., for authentication) and is therefore not suitable for the identification of unknown target nucleic acid molecules or a mixture of more than one target nucleic acid molecule.

An alternative method for the amplification and detection of molecular taggants is provided by U.S. Pat. No. 8,735,327, which describes a DNA taggant system that attempts to address the problems of taggant layering and identification using a primer site encoding system that applies combinatorial mathematical approaches to decode amplification reaction products. Accordingly, primer sites are selected from a library of non-hybridising sequences that correspond to a bit value and string position. Taggants are decoded by screening with all primer pair combinations in the library, and Wₙ is decoded from the resulting network graph of positive PCR reactions (Gᵤ). Samples become un-decodable, however, when Gᵤ contains overlapping sub-graph cliques that are not contained in the set of taggant present in a sample, Wᵤ.

In U.S. Pat. No. 8,735,327 the use of strings of primer sites to encode information limits the information storage capacity due to constraints on the coding string length. Furthermore, reliance on primer sites to encode information means that a large number of non-hybridizing primer sequences are required and many hundreds of screening reactions must be performed to decode the information within the taggants. For example, a binary system (s=2) of length n=5 has a library size of sⁿ=2⁵=32 taggants requires (n(n−1)s²)/2=40 reactions to decode (i.e. 40 primer pair combinations). Furthermore, the method of Macula is also incompatible with deeply layered applications due to restrictions on the number of taggants that can be mixed and subsequently decoded. For example, the n5-s2 binary system has a maximum layering depth of ns−n+1=6. The layering depth (ie. mixing capacity) may be increased through the use multiple libraries or ternary or quaternary encoding systems. However, both of these approaches dramatically increase the number of screening reactions required. For example, an n5-s5 system has a library size of 3,125 taggants, but requires 625 reactions to decode and has a maximum mixing limit of 21 taggants. As such, all existing taggant systems remain out of reach for identification applications that require a mixing limit exceeding approximately 20 taggants. The large number of samples needed, as required by the Macula approach, is also not compatible with forensic and trace DNA recovery applications.

Although it is relatively simple to tag matter with a molecular taggant, the taggant is only of value where the nucleic acid sequence can be amplified and subsequently decoded to identify and/or authenticate the taggant. However, existing nucleotide taggant systems remain cumbersome, impractical, and expensive for identification purposes and are not adapted to be efficiently decoded in a manner that allows for the identification of a subset of unknown taggants (and taggant layering). The large number of samples required to identify a product is also not conducive to low-copy number and forensic applications.

Accordingly, there remains a need for methods that allow for the identification of molecular taggants and taggant layering.

### SUMMARY

In an aspect disclosed herein, there is provided a method for high fidelity amplification of two or more target nucleic acid sequences in a mixture thereof, wherein each of the two or more target nucleic acid sequences are flanked by a first primer site and a second primer site, wherein the amplification comprises thermocycling comprising a melting phase, an annealing phase and an extension phase, the method comprising using a first primer complementary to each of the first primer sites and a second primer complementary to each of the second primer sites, wherein each of the first and second primers comprise at least one locked nucleic acid (LNA) and wherein an elevated temperature is used during the annealing phase of the thermocyling such that, during the annealing phase, there is substantially no annealing of nucleic acid sequences other than of the first and second primers to the first and second primer sites, respectively, wherein one or more or all of the following apply,

i) the two or more target nucleic acid sequences are amplified in a single thermocycling reaction;

ii) the two or more target nucleic acid sequences encode non-biological information; or

iii) each of the two or more target nucleic acids are flanked by a common first primer site and a common second primer site.

In another aspect disclosed herein, there is provided a method of tracing a product to its origin, the method comprising:

(a) providing a product to which at least one nucleic acid sequence has been incorporated, wherein the at least one nucleic acid sequence is flanked by a first primer site and a second primer site;
(b) optionally recovering the at least one nucleic acid sequence from the product;
(c) amplifying the at least one nucleic acid sequence by high fidelity amplification comprising thermocycling using a first primer complementary to the first primer site and a second primer complementary to the second primer site, wherein the first and second primers each comprise at least one locked nucleic acid (LNA), wherein the thermocycling comprises a melting phase, an annealing phase and an extension phase, and wherein an elevated temperature is used during the annealing phase of the thermocycling such that, during the annealing phase, there is substantially no annealing of nucleic acid sequences other than of the first and second primers to the first and second primer sites, respectively; and
(d) identifying the at least one nucleic acid sequence amplified in step (c);
wherein the sequence and/or length of the at least one nucleic acid sequence identified in step (d) is indicative of the origin of the product.

In another aspect disclosed herein, there is provided a kit comprising a first component and a second component, wherein the first component comprises a library of two or more nucleic acid sequences, wherein each of the two or more nucleic acid sequences is flanked by a common first primer site and a common second primer site, and wherein the second component comprises a first primer complementary to the first primer site and a second primer complementary to the second primer site, and wherein the first and second primers each comprise at least one locked nucleic acid (LNA).

In another aspect disclosed herein, there is provided a method for high fidelity amplification of a target nucleic acid sequence flanked by a first primer site and a second primer site, wherein the amplification comprises thermocycling comprising a melting phase, an annealing phase and an extension phase, the method comprising using a first primer complementary to the first primer site and a second primer complementary to the second primer site, wherein the first and second primers each comprise at least one locked nucleic acid (LNA) and wherein an elevated temperature is used during the annealing phase of the thermocycling such that, during the annealing phase, there is substantially no annealing of nucleic acid sequences other than of the first and second primers to the first and second primer sites, respectively.

In another aspect disclosed herein, there is provided a method for high fidelity amplification of two or more target nucleic acid sequences in a mixture thereof, wherein each of the two or more target nucleic acid sequences are flanked by a first primer site and a second primer site, wherein the amplification comprises thermocycling comprising a melting phase, an annealing phase and an extension phase, the method comprising using a first primer complementary to each of the first primer sites and a second primer complementary to each of the second primer sites, wherein each of the first and second primers comprise at least one locked nucleic acid (LNA) and wherein an elevated temperature is used during the annealing phase of the thermocyling such that, during the annealing phase, there is substantially no annealing of nucleic acid sequences other than of the first and second primers to the first and second primer sites, respectively.

In another aspect disclosed herein, there is provided a method of tracing a product to its origin, the method comprising:

(a) providing a product to which at least one nucleic acid sequence has been incorporated, wherein the at least one nucleic acid sequence is flanked by a first primer site and a second primer site;
(b) recovering the at least one nucleic acid sequence from the product;
(c) amplifying the recovered at least one nucleic acid sequence by high fidelity amplification comprising thermocycling using a first primer complementary to the first primer site and a second primer complementary to the second primer site, wherein the first and second primers each comprise at least one locked nucleic acid (LNA), wherein the thermocycling comprises a melting phase, an annealing phase and an extension phase, and wherein an elevated temperature is used during the annealing phase of the thermocycling such that, during the annealing phase, there is substantially no annealing of nucleic acid sequences other than of the first and second primers to the first and second primer sites, respectively; and
(d) identifying the at least one nucleic acid sequence amplified in step (c);
wherein the sequence and/or length of the at least one nucleic acid sequence identified in step (d) is indicative of the origin of the product.

### BRIEF DESCRIPTION OF THE FIGURES

FIG. 1 is a schematic representation of an example of the use of taggant layering (mixing) for supply chain tracing and product identification. In this example, seven product precursors are marked with seven oligonucleotide taggants (1-7). The intermediate and final combined products contain multiple oligonucleotide taggants that are indicative of the product's origin. For the UniKey-Tag embodiments disclosed in this document, there is essentially no limit to layering depth/mixing limit (millions). In any sample, all oligonucleotide taggants can be recovered and amplified in one reaction with annealing temperature discrimination polymerase chain reaction (ATD PCR).

FIG. 2 is a schematic representation of the experimental procedure for ATD PCR used for random access capabilities in oligonucleotide based archival data storage systems. The archived data is comprised of a pool (P) of oligonucleotide fragments (τ) that encode the three pictures files (a, b, c). In the example, each set of fragments used to encode a particular picture file contains a pair of primer site sequences that are common to that file. Random access data recovery is performed using a universal set of LNA-primers to recover the file of interest. For example, UPFb and UPRb will recover picture (b). The higher binding temperature of ATD PCR also allows greater encoding flexibility in the variable region by reducing Watson-Crick binding constraints that may lead to heterodimer formation and cross-hybridised PCR products.

FIG. 3 shows how LNA-primers can be used to decrease letter length (1) and therefore increase the codeword string length (n) in primer site encoded systems (UniKey-Tag 2 systems). In primer site encoded systems, Watson-Crick DNA binding biochemistry typically requires that (a) the letter length is about 20-30 bp, which limits the string length to a maximum of n=5 for a variable coding region of 100 bp. The high binding affinity of LNA-primers allows (b) a reduction in primer length, and therefore letter length, which allows an inversely proportional increase in the string length n for any set variable region length v. An increase in n increases the information storage capacity of the variable region and the size of the taggant library available wₙ (ie. number of codewords available).

FIG. 4 is a graphical representation that shows how annealing temperature discrimination PCR (ATD PCR) minimizes cross-fragment hybridisation. The diagrams show amplification reaction products of fragments that contain common primer site sequences using conventional PCR and ATD PCR. In (a), a mixture of denatured single stranded fragments with common primer sites but different variable regions (V_1 and V_2) are shown. During PCR, ssDNA fragments are cooled to allow primers to bind to the exposed strands. In conventional PCR (b) primer-fragment and fragment-fragment annealing occurs at a similar temperature, which results in (b i) cross-fragment priming, (b ii) cross-fragment hybridization, and (b iii) non-specific hybrid fragment annealing and elongation. These processes ultimately result in PCR products that contain (b iv) fragment hybrids of variable origin and length. Conversely in ATD PCR (c) the amplification reaction annealing temperature is set to permit LNA primer-fragment interactions but prevent fragment-fragment interactions. The LNA primer-fragment complex is ideally designed to anneal at a temperature >5° C. higher than fragment-fragment complementary primer site interactions. This prevents cross-tag hybridization. Abbreviations include: cap region (Cp), universal forward primer sequence (PF), universal forward primer complementary sequence (PF_{c}), universal reverse primer sequence (PR), universal reverse primer complementary sequence (PR_{c}), variable region x (V_x), variable region x complementary sequence V_x (Vc_x).

FIG. 5 shows how common symbol sequences used in different codewords may result in heterodimer formation and cross-fragment hybridisation during conventional PCR. In (a) the crumb sequence (equivalent to a binary byte) for the symbol 27 is used in two different codewords, which in (b) permits cross-fragment priming and hybridisation. ATD PCR allows the annealing temperature to be set sufficiently high to discriminate against these interactions. This reduces Watson Crick DNA binding constraints and permits greater encoding flexibility which is particularly advantageous for encoding non-biological information into DNA, since almost all encoding systems use common symbol sequences.

FIG. 6 shows the thermal cycle for conventional PCR and ATD PCR. The PCR thermal cycle steps shown include: (a) initial activation step for hot start polymerases, (b) dsDNA strand denaturation (c1) high temperature LNA-primer fragment annealing used in ATD PCR (c2) low-temperature conventional primer (and fragment-fragment) annealing, (d) polymerase mediated strand elongation. Steps (b) to (d) are repeated n times for exponential amplification, step (e) is a final elongation phase and in step (f) the PCR product is cooled for storage. The LNA containing primers were designed such that the temperature difference (Δ_{TA}) between (c1) and (c2) was at least 5° C. in ATD PCR experiments to prevent cross-fragment hybridisation.

FIG. 7 is a generic design of a double-stranded taggant. The diagram shows a generic dsDNA taggant comprised of a template and complementary strand. Locations marked on the template strand are (left to right): optional capping region (Cp), region identical to the forward primer sequence (PF), variable encoding region (V_x), region complementary to the reverse primer (PRO, and optional region complementary to the capping region on the opposing complementary strand (Cp_{c}). The subscript ‘c’ indicates ‘complementary to’. The regions identified by lowercase letters have length units in base pairs (bp) and include: fragment length (k), capping length (j), primer site length (p), variable region length (v) and symbol/letter length (l). The number of letters in the variable region string is n, where n=v/l.

FIG. 8 is a schematic representation of (a) a target nucleic acid sequence whereby the sequence of nucleotides is indicative of origin (UniKey-Tag 1 system); and (b) a target nucleic acid whereby the length of the target sequence is indicative of origin (UniKey-Tag 2 system). In the UniKey-Tag 1 system, (a), each letter L in the codeword string n is encoded by ≥1 nucleotide (l≥1), and n is decoded by sequencing. In the UniKey-Tag 2 system, (b), each primer pair encodes a particular letter L_{A}, L_{B}, L_{C} (i.e. PF(A), PF(B), PF(C)) and the position of L in string n is determined by the length of v which is variable (//). The codeword n is decoded by ATD PCR amplification and product length separation. For all taggant types: k is the length of the oligonucleotide fragment (bp), j is the length of the optional 3′ and 5′ cap (bp), p is the length of the forward and reverse primer (bp), v is the length of the variable region (bp), l is the length of each letter (bp) in a codeword string of n letters. Regions within the taggants are: capping region (Cp), universal forward primer site (UPF), universal reverse primer site (UPR), and variable region (V_x). In (b), PF(A, B, C) are primer sites specific to the letters A, B, C. The subscript ‘c’ denotes ‘complementary to.’

FIG. 9 shows ADT PCR product preparation for sequencing by synthesis (Illumina platform). The diagrams show sample preparation steps for next generation sequencing. In the first step (a) adapter sequences are ligated to the oligonucleotide tags using the primer regions that contain LNAs from ATD PCR. The second adapter sequence is added to opposite end of each strand (b), such that the template and complementary strands now include both 5′ and 3′ adapter sequences. The final products for Illumina sequencing (c) only contain conventional nucleotides since LNA containing regions are eliminated during ligation steps. This occurs because the adapter sequences do not contain LNAs. Abbreviations are: In (locked nucleotides), cv (conventional nucleotides), UPF (universal forward primer), UPR (Universal reverse primer), V_1 (variable region 1), subscript ‘c’ denotes a complementary region, and P7 and P5 are adapter sequences given in the Illumina protocol.

FIG. 10 shows how multiple samples can be barcoded with LNA primers and ATD PCR for parallel sequencing. In this example a unique barcode identifier sequence is added to the 5′ end of the LNA primer to identify sample (either the forward or reverse primer may be used). The samples are amplified by ATD PCR, pooled together, sequenced in parallel and then decoded. In this example, (a) Sample 1 is labelled by barcode 1 and contains fragments encoded with variable region 1 (V_1), in (b) Sample 2 is labelled with barcode 2 and contains fragments encoded with variable regions 2 and 3 (V_2, V_3) and (c) shows the pooled barcoded samples prepared for parallel sequencing.

FIG. 11 is another illustrative example of the UniKey-Tag 2 system: (a) multiple taggants of variable length encode each letter L in the coding string n, and (b) diagram of amplified products decoded by gel electrophoresis fragment length separation. The diagram (a) shows a group of eight n₈-s₃ oligonucleotide taggants, where the length of each taggant encodes the position of the symbol in the string n and each primer pair encodes a letter L in the set S={A, B, C}, ie. s=3. A final combined product (b) can be marked with two or more sets of layered taggants at the level of the individual taggant τ, the set of taggants for each letter L, and the set of letters in the alphabet S. The diagram (c) shows amplification products that would be generated from the combined product in (b) separated by gel electrophoresis. Fragments are decoded by noting the migration distance (which is inversely proportional to fragment length) and the gel lane (letter). This effectively forms a two-dimensional codeword on the gel, where each lane represents a different letter (x-axis) and the migration distance of the DNA bands (y-axis) represents the position of the letter in the codeword. Note that two different letters can occupy the same position in the codeword. As each L in the set S is decoded simultaneously using ATD PCR, only three screening reactions are required for the 11 taggants in this example.

FIG. 12 shows photographs of electrophoresis gels comparing the amplification products of (a) ATD PCR and (b) conventional primer PCR over variable annealing temperature range (4, 2, and 0° C. below the design temperature). For both protocols, amplification was performed on a prepared standard solution containing 25 pM of OligoTag_1-4_Ser1 taggants in Table 1. These taggants have the same post-PCR amplification length of 74 bp and identical forward and reverse primer sites. The UniKey-Tag protocol (a) shows no visual evidence of cross-fragment hybridisation, with a single clear band present for annealing temperatures (AT) of 65-69° C. In contrast, conventional recovery and amplification techniques (b) show smearing and striations over an annealing temperature (AT) range of 49-53° C., which is indicative of cross-taggant priming and amplification. The faint band at 20 bp is overloaded primer that has not been incorporated into PCR product. For both (a) and (b) the lanes are as follows: (1) Hyperladder 25, (2) AT at 4° C. below design Tₘ, (3) AT at 2° C. below design Tₘ, and (4) AT at design Tₘ.

FIG. 13 is a photograph of an electrophoresis gel showing the amplification products of a mixture of universal primer site encoded fragments of different length using the ATD PCR protocol (lanes 3 and 5) and conventional PCR (lanes 2 and 4) with variable cycle times. For both protocols, amplification was performed on a prepared standard solution containing 25 pM of: OligoTags_1-4_Ser1, OligoTags_9-12_Ser1, and OligoTags_17-20_Ser1 (sequences are provided in Table 1). These fragments have post-amplification length of 74, 64, and 54 bp, respectively. For lanes 2 and 3, amplification was performed with longer annealing and elongation times (15s, and 20s respectively) and in lanes 4 and 5 the standard thermo-cycle protocol was used (5s and 10s respectively). Lanes 1 and 3 show the products of conventional PCR at annealing temperature (AT)=51° C., and lanes 3 and 5 show the products of the ATD PCR at the designed AT=69° C. (Δ_{AT}=18° C.). The smears and striations in lanes 2 and 4 indicate that cross-fragment hybridisation occurred when conventional PCR was used. In contrast, lanes 3 and 5 show three distinct bands indicating that ATD prevented cross-fragment hybridisation. The control for the UniKey-Tag protocol is shown in lane 6. The faint bands at 20 bp are excess primer that was not incorporated into PCR product.

FIG. 14 is a photograph of an electrophoresis gel showing the amplification products of samples taken from recovered bullets after firing using ATD PCR methodology (Example 4). Ammunition cartridges were separated into three groups and marked with UniKey-Tags: OligoTag_4, 12, and 20_Ser1 (see Example 1). These taggants have common forward and reverse primer sequences and post-amplification lengths of 74, 64, or 54 bp., respectively. The presence of multiple defined bands indicates (a) the transfer of taggants onto successive cartridges loaded into the magazine, (b) that cross-tag hybridisation did not occur during amplification, and (c) the viability of UniKey-Tag technology in the field for the purpose of ammunition tracing. The lanes are as follows: (1) Hyperladder 25; recovered bullets that were tagged with (2) OligoTag_4_Ser1, (3) OligoTag_12_Ser1, (4) OligoTag_20_Ser1, (5) OligoTag_4_Ser1, (6) OligoTag_12_Ser1, (7) OligoTag_20_Ser1, (8) OligoTag_4_Ser1, (9) OligoTag_12_Ser1, (10) OligoTag_20_Ser1, (11) OligoTag_4_Ser1, (12) OligoTag_12_Ser1, (13) OligoTag_20_Ser1, (14) OligoTag_4_Ser1; and (15) Hyperladder 25.

FIG. 15 is a diagram showing how Hamming (8,4,4) encoded fragments were prepared for Series 2 experiments. Each Ham(8,4,4) crumb is comprised of data nucleotides (d₀-d₃ in blue) and parity nucleotides (p₀-p₃ in black). Codewords of length n6 were assembled from the crumb library (Table 3) flanked by universal forward and reverse complementary primer sites (UFPS and URCPS, respectively) in pink (sequences provided in Table 4). Candidate codewords were selected after screening for high complementarity against the Kingdom Metazoa (E≤0.1) and for CG-rich regions.

FIG. 16 is a schematic representation of the ammunition tracing experimental arrangement for the five-point taggant recovery analysis. The shooter was positioned 10 m from the target consisting of a section of biological material (supermarket pork belly) backed with plywood and sandbags. The five taggant recovery points labeled are the (a) hand; (b) firearm; (c) spent cartridge cases; (d) bullet entry point; and (e) bullet recovered from the sandbags. Results of the combined Series 1 experiments (a and b) for the UniKey-Tag 2 system are shown (fragment length separation results). The y-axis units are percentage, n=70.

FIG. 17 is a graphical representation of the combined results for the Series 1 (UniKey-Tag 2 system) ammunition tracing experiments (a) and (b). The y-axis shows the frequency that the expected fragment was detected (%) for each of the five recovery points listed on the x-axis.

FIG. 18 shows the results of the accelerated degradation experiments for the DNA-taggant fixing solutions given in Table 10.

FIG. 19 shows the results for Series 1 (9 mm handgun) and Series 2 (.22 and .207 calibre firearms) ammunition tracing experiments where samples were decoded by sequencing (ie. the UniKey-Tag 1 system). This includes (a) the frequency that the expected DNA trace was detected in all samples. Expected signal (ES) to noise (N) ratios, based on sequencing record count, are given for (b) case, (c) entry point and (d) recovered bullet samples respectively. The left y-axis shows ES and N values normalized to mean ES, the right y-axis shows mean ES/N. The probability of ES as a function of record rank is shown in (e). Here, nₛ=no. samples, nᵣ=no. sequencing records, nₜ=no. traces.

FIG. 20 is a photograph of electrophoresis gels showing ATD PCR products from ammunition cartridge cases for Series 1 (a) experiment a and (b) experiment b (UniKey-Tag 2).

FIG. 21 is a photograph of electrophoresis gels showing ATD PCR products from entry site samples for Series 1 (a) experiment a and (b) experiment b (UniKey-Tag 2).

FIG. 22 is a photograph of electrophoresis gels showing ATD PCR products from bullets for Series 1 (a) experiment a and (b) experiment b (UniKey-Tag 2).

### KEY TO THE SEQUENCE LISTING

| | |
|---|---|
| SEQ ID NO: 1 | OligoTag_1T_Ser1 |
| SEQ ID NO: 2 | OligoTag_1C_Ser1 |
| SEQ ID NO: 3 | OligoTag_2T_Ser1 |
| SEQ ID NO: 4 | OligoTag_2C_Ser1 |
| SEQ ID NO: 5 | OligoTag_3T_Ser1 |
| SEQ ID NO: 6 | OligoTag_3C_Ser1 |
| SEQ ID NO: 7 | OligoTag_4T_Ser1 |
| SEQ ID NO: 8 | OligoTag_4C_Ser1 |
| SEQ ID NO: 9 | OligoTag_5T_Ser1 |
| SEQ ID NO: 10 | OligoTag_5C_Ser1 |
| SEQ ID NO: 11 | OligoTag_6T_Ser1 |
| SEQ ID NO: 12 | OligoTag_6C_Ser1 |
| SEQ ID NO: 13 | OligoTag_7T_Ser1 |
| SEQ ID NO: 14 | OligoTag_7C_Ser1 |
| SEQ ID NO: 15 | OligoTag_8T_Ser1 |
| SEQ ID NO: 16 | OligoTag_8C_Ser1 |
| SEQ ID NO: 17 | OligoTag_9T_Ser1 |
| SEQ ID NO: 18 | OligoTag_9C_Ser1 |
| SEQ ID NO: 19 | OligoTag_10T_Ser1 |
| SEQ ID NO: 20 | OligoTag_10C_Ser1 |
| SEQ ID NO: 21 | OligoTag_11T_Ser1 |
| SEQ ID NO: 22 | OligoTag_11C_Ser1 |
| SEQ ID NO: 23 | OligoTag_12T_Ser1 |
| SEQ ID NO: 24 | OligoTag_12C_Ser1 |
| SEQ ID NO: 25 | OligoTag_13T_Ser1 |
| SEQ ID NO: 26 | OligoTag_13C_Ser1 |
| SEQ ID NO: 27 | OligoTag_14T_Ser1 |
| SEQ ID NO: 28 | OligoTag_14C_Ser1 |
| SEQ ID NO: 29 | OligoTag_15T_Ser1 |
| SEQ ID NO: 30 | OligoTag_15C_Ser1 |
| SEQ ID NO: 31 | OligoTag_16T_Ser1 |
| SEQ ID NO: 32 | OligoTag_16C_Ser1 |
| SEQ ID NO: 33 | OligoTag_17T_Ser1 |
| SEQ ID NO: 34 | OligoTag_17C_Ser1 |
| SEQ ID NO: 35 | OligoTag_18T_Ser1 |
| SEQ ID NO: 36 | OligoTag_18C_Ser1 |
| SEQ ID NO: 37 | OligoTag_19T_Ser1 |
| SEQ ID NO: 38 | OligoTag_19C_Ser1 |
| SEQ ID NO: 39 | OligoTag_20T_Ser1 |
| SEQ ID NO: 40 | OligoTag_20C_Ser1 |
| SEQ ID NO: 41 | OligoTag_1T_Ser2 |
| SEQ ID NO: 42 | OligoTag_1C_Ser2 |
| SEQ ID NO: 43 | OligoTag_2T_Ser2 |
| SEQ ID NO: 44 | OligoTag_2C_Ser2 |
| SEQ ID NO: 45 | OligoTag_3T_Ser2 |
| SEQ ID NO: 46 | OligoTag_3C_Ser2 |
| SEQ ID NO: 47 | OligoTag_4T_Ser2 |
| SEQ ID NO: 48 | OligoTag_4C_Ser2 |
| SEQ ID NO: 49 | OligoTag_5T_Ser2 |
| SEQ ID NO: 50 | OligoTag_5C_Ser2 |
| SEQ ID NO: 51 | OligoTag_6T_Ser2 |
| SEQ ID NO: 52 | OligoTag_6C_Ser2 |
| SEQ ID NO: 53 | OligoTag_7T_Ser2 |
| SEQ ID NO: 54 | OligoTag_7C_Ser2 |
| SEQ ID NO: 55 | OligoTag_8T_Ser2 |
| SEQ ID NO: 56 | OligoTag_8C_Ser2 |
| SEQ ID NO: 57 | OligoTag_9T_Ser2 |
| SEQ ID NO: 58 | OligoTag_9C_Ser2 |
| SEQ ID NO: 59 | OligoTag_10T_Ser2 |
| SEQ ID NO: 60 | OligoTag_10C_Ser2 |
| SEQ ID NO: 61 | FwdPrimer_Ser1 |
| SEQ ID NO: 62 | RevPrimer_Ser1 |
| SEQ ID NO: 63 | LNA_FwdPrimer_Ser1 |
| SEQ ID NO: 64 | LNA_RevPrimer_Ser1 |
| SEQ ID NO: 65 | FwdPrimer_Ser2 |
| SEQ ID NO: 66 | RevPrimer_Ser2 |
| SEQ ID NO: 67 | LNA_FwdPrimer_Ser2 |
| SEQ ID NO: 68 | LNA_RevPrimer_Ser2 |

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

The present application claims priority from AU 2016902892 filed 22 Jul. 2016, the entire contents of which are incorporated herein by reference.

Unless otherwise indicated, the molecular biology techniques described herein are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996) and F. M. Ausubel et al. (editors), Current Protocols in Molecular Biology, Greene Pub. Associates and Wiley-Interscience (1988, including all updates until present).

All publications mentioned in this specification are herein incorporated by reference in their entirety.

It must be noted that, as used in the subject specification, the singular forms “a”, “an” and “the” include plural aspects unless the context clearly dictates otherwise. Thus, for example, reference to “a fragment” includes a single fragment, as well as two or more fragments.

Nucleic acids are ideal molecular tags due to their inherent stability, information density and ease of synthesis. Non-biological information may also be encoded into nucleic acid sequences and decoded using routine molecular biology techniques that are known in the art. Molecular tags comprising nucleic acids may be incorporated into a product or its packaging to allow for the identification, authentication and tracing of the product or its packaging. The information encoded by the molecular taggant can be used for any suitable purpose, illustrative examples of which include the place of origin and the date of manufacture.

Although it is relatively simple to tag matter with molecular tags, the tag is only of limited value unless it can be identified. Previously developed nucleic acid taggant systems cannot efficiently detect and decode unknown tags or an unknown mixed subset of tags from a larger pool of tags. This is largely due to a reliance on specific primer-tag combinations that require independent amplification reactions to authenticate such tags.

The present disclosure is predicated on the inventor's finding that LNA containing primers may be used to introduce a selective parameter ‘annealing temperature’ to discriminate against fragment-fragment interactions during amplification of a plurality of nucleic acids with (a) common primer site sequences and/or (b) common subsequences between the primer sites, and therefore prevent cross-fragment hybridisation.

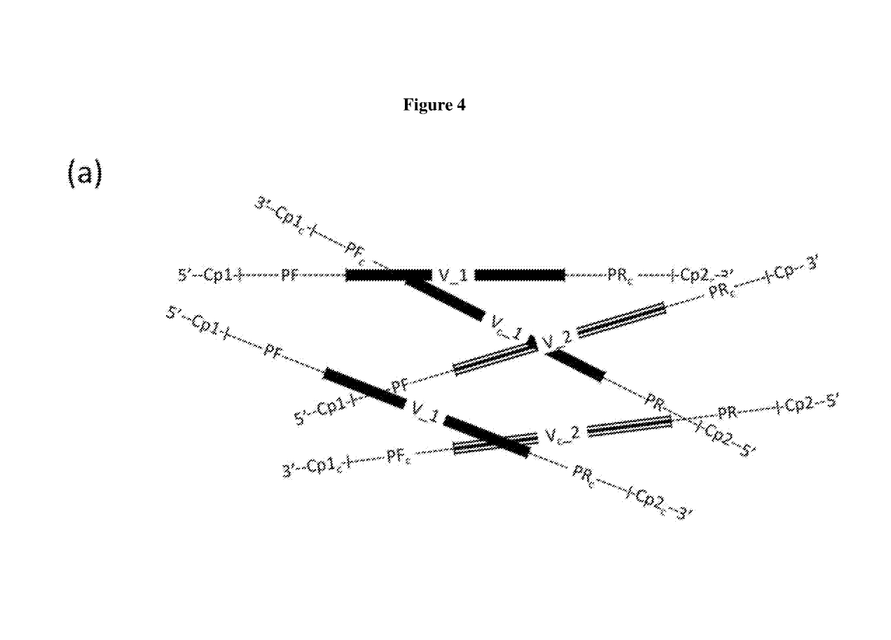

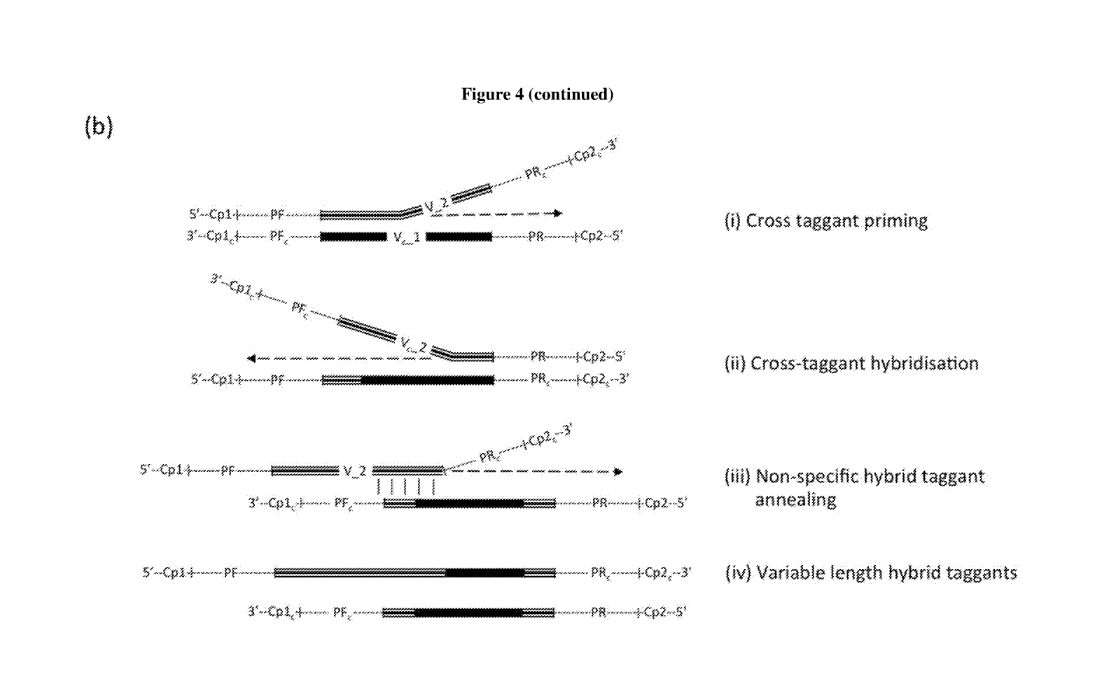

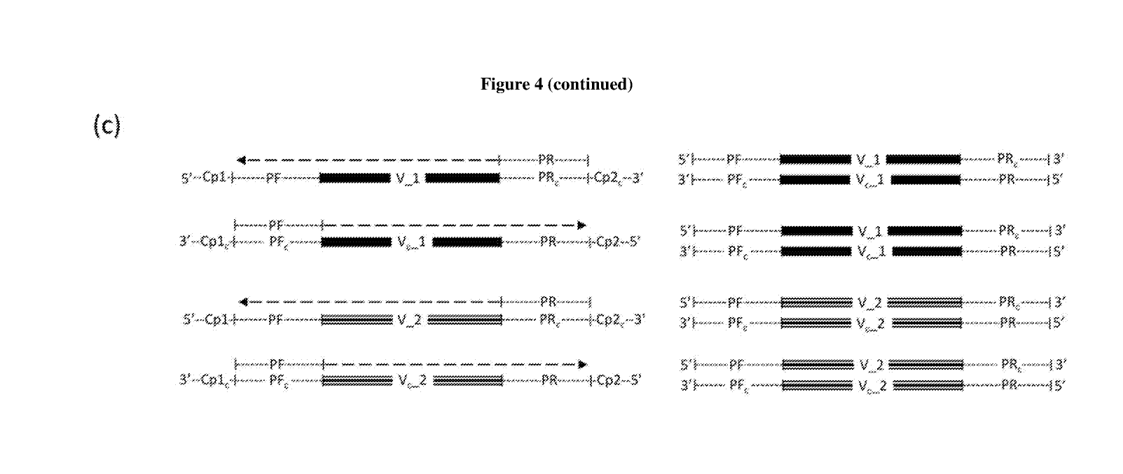

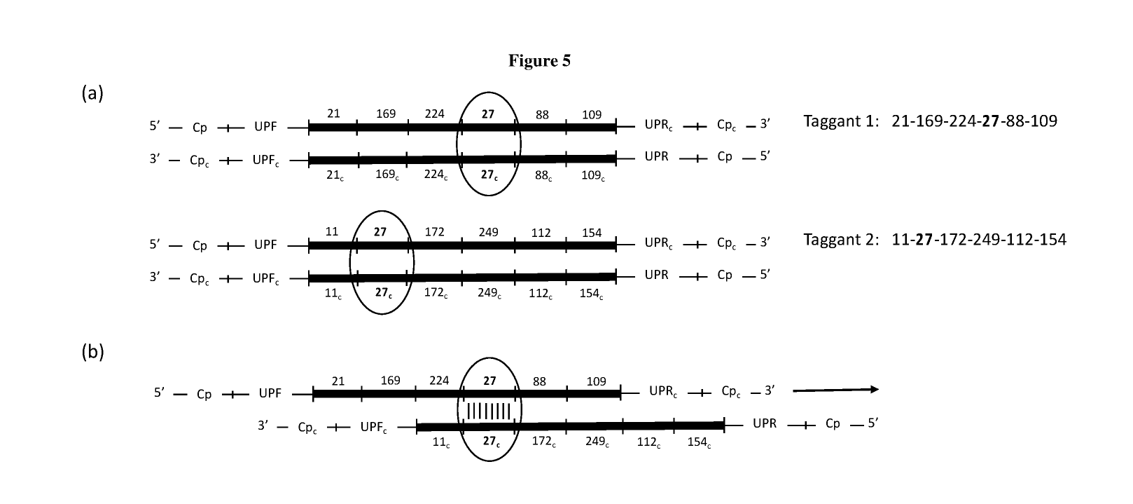

By using LNA-primers, the annealing temperature of a thermocycling amplification reaction can be elevated to allow for the formation of LNA primer-fragment complexes, but discriminate against the formation of complementary conventional nucleotide complexes (via universal primer sites or common symbol subsequences) and non-specific complexes that would otherwise occur at lower annealing temperatures. This method is particularly useful for the simultaneous amplification of multiple tags comprising different nucleic acid sequences where unwanted specific and non-specific fragment-fragment cross-hybridization is problematic. For example, specific cross-hybridisation is a problem when the target pool of nucleic acids contain (a) common primer sequences, or (b) common subsequences between the primer sites (See also FIGS. 4 and 5). Here, specific means that the unwanted interactions occur between two subsequences that are substantially complementary.

Accordingly, in an aspect disclosed herein, there is provided a method for high fidelity amplification of a target nucleic acid sequence flanked by a first primer site and a second primer site, wherein the amplification comprises thermocycling comprising a melting phase, an annealing phase and an extension phase, the method comprising using a first primer complementary to the first primer site and a second primer complementary to the second primer site, wherein the first and second primers each comprise at least one locked nucleic acid (LNA) and wherein an elevated temperature is used during the annealing phase of the thermocycling such that, during the annealing phase, there is substantially no annealing of nucleic acid sequences other than of the first and second primers to the first and second primer sites, respectively.

### Target Nucleic Acid Sequences and Tags

It is to be understood that the methods disclosed herein are suited to the high fidelity amplification of any target nucleic acid sequence. The terms “target nucleic acid”, “target nucleic acid sequence”, “target nucleotide sequence”, “target nucleic acid molecule”, “nucleic acid”, “nucleic acid sequence”, “nucleotide sequence”, “nucleic acid molecule”, “oligo”, “oligonucleotide”, “nucleic acid fragment”, “fragment” and the like, are understood to mean a covalently linked sequence of nucleotides in which the 3′ position of the phosphorylated pentose of one nucleotide is joined by a phosphodiester group to the 5′ position of the pentose of the next nucleotide and in which the nucleotide residues are linked in specific sequence; i.e. a linear order of nucleotides. The target nucleic acid can be single stranded or double stranded.

Target nucleic acid sequences may be naturally-occurring (e.g., isolated from a natural or transgenic organism) or may be artificial (i.e., synthesized). Target nucleic acids may comprise natural or non-natural nucleotides, or a combination of both. Natural nucleotides typically refer to the five naturally occurring bases—adenine, thymine, guanine, cytosine and uracil. In an embodiment disclosed herein, the target nucleic acid sequence comprises synthetic nucleotides. Synthetic nucleotides have some advantages over naturally-occurring nucleotides, such as improved stability, solubility and resistance to nuclease activity, heat and/or ultraviolet radiation (UV). In some embodiments, non-natural or synthetic nucleic acids include those incorporating inosine bases and derivatized nucleotides, such as 7-deaza-2′deoxyguanosine, methyl- or longer alkyl-phosphonate oligodeoxynucleotides, phosphorothioate oligodeoxynucleotides, and alpha-anomeric oligodeoxynucleotides.

In an embodiment, one or more of the target nucleic acids comprise a nucleic acid sequence selected from SEQ ID NOs: 1 to 60.

As noted elsewhere herein, nucleic acids are ideal molecular tags due to their inherent stability, information density and ease of synthesis. The terms “tag” and “taggant” are used interchangeably herein to mean a nucleic acid molecule that can be attached, applied or otherwise incorporated into or onto a product to allow for subsequent identification, authentication and/or tracing of the product by detection of the nucleic acid tag, whether the tag is detected on the product to which it was attached, applied or otherwise incorporated, or on a surface to which the tagged product has come in contact (e.g., the surface of an entry point of a tagged projectile, such as a bullet fired from a handgun or rifle).

Thus, the terms “tag” and “taggant” are used interchangeably herein with “nucleic acid”, “nucleic acid sequence”, “nucleotide sequence”, “nucleic acid molecule”, “target nucleic acid”, “target nucleic acid sequence”, “target nucleotide sequence”, “target nucleic acid molecule”, “oligo”, “oligonucleotide”, “nucleic acid fragment”, “fragment” and the like. These terms, collectively, are to be understood to include both single stranded (ss) and double stranded (ds) forms of the aforementioned.

Where the target nucleic acid sequences are nucleic acid tags applied, attached or otherwise incorporated in a product, article or substance, in some embodiments at least a sample of the nucleic acid will need to be recovered for subsequent amplification by the methods disclosed herein. In some embodiments, recovery of the nucleic acid tag from the product is not necessary. For example, when the product is a pharmaceutical product, then the product can be directly dissolved into the amplification reaction mixture. Suitable methods for the recovery of a nucleic acid tag from a product or substance will be familiar to persons skilled in the art, illustrative examples of which include extracting the tag from the product with either distilled water or a buffered solution. Physiological pH is typically preferred, as acidic or basic pH levels may degrade the nucleic acid tags. Where the nucleic acid tag is attached, applied or otherwise incorporated into a charged product or substance, the product or substance may require a wash in high molarity salt buffer to act as an ion exchanger with the electrostatically bound nucleic acid tag. Ionic or non-ionic detergents may also be helpful to remove nucleic acids from surfaces or from complex mixtures. Phenol based extractions or phenol/chloroform extractions can also be used to recover nucleic acid from complex biological substances or from oil-based substances.

In some embodiments, the recovered nucleic acid tags can be concentrated by standard techniques known to persons skilled in the art, such as precipitation with alcohol, evaporation, or microfiltration.

### Information-Encoded Nucleic Acid Sequences

According to the methods described herein, the elevated annealing temperature during thermocycling substantially reduces the occurrence of interactions between universal primer site encoded taggants in a mixture. In some embodiments, there is substantially no annealing of nucleic acid sequences other than of the first and second primers to the first and second primer sites respectively. Thus, the elevated annealing temperature reduces the probability of cross-taggant heterodimer formation. This is particularly important to address three problems specific to the application described here: (1) conventional extended cycle PCR amplification (>20 cycles) of universal primer site encoded libraries is required to produce a sufficient amount of product for sequencing but results in cross-fragment hybridisaiton, (2) different taggant codewords that contain the same symbol encoded by a common subsequence (such as in FIG. 5) are more likely to form cross-hybridised products if conventional PCR is used, and (3) the low annealing temperature of conventional PCR imposes more stringent biochemical constraints on the sequences available to encode information due to non-specific heterodimer formation (for eg., GC rich sequences are problematic).

In an embodiment disclosed herein, the target nucleic acid sequence encodes non-biological information. The phrase “non-biological information” typically means that the sequence is not designed to perform a function when expressed in a living cell. Thus, nucleic acid sequences encoding non-biological information do not comprise an open reading frame encoding a functional polypeptide. Therefore, in some embodiments, the tag will comprise, consist or consist essentially of a nucleic acid sequence that does not exist in a naturally occurring organism.

In some embodiments, information can be encoded into target nucleic acid sequences using nucleotides, or subsets of nucleotides, as characters or symbols (i.e., alphanumeric characters, special characters, etc.) or binary codes (i.e., ones and zeros). For example, the basic nucleotides A (adenine), G (guanine), C (cytosine) and T (thymine) for DNA (or A, G, C and U (uracil) for RNA) allow for vast amounts of information to be stored in relatively short nucleic acid sequences, wherein each nucleotide base, or string of nucleotide bases, represent a character or symbol (ie. l=1 bp), such as a letter of an alphabet or, in the context of a binary code, ones and zeros.

In an embodiment disclosed herein, the information encoded by the target nucleic acid provides the address in a directory (e.g., a computer database) where additional information is stored. In another embodiment, the information is encoded directly into the target nucleic acid so that it can be decoded/deciphered by anyone who knows the method of encoding/encryption that was used.

In an embodiment, the taggant encodes a binary code, wherein each nucleotide A, G, C and T (or U) represents a “zero” or a “one”. In an illustrative example, nucleotides A and G represent “ones” and nucleotides C, T and U represent “zeros”. Thus, a binary code may be encoded into a taggant by the suitable arrangement of nucleotides. The binary code “010011010” can therefore be encoded by the nucleic acid sequences “CATTAGTAC”, “TGCCGGCAT”, “AGCTGAUAC” and so on. Conversely, nucleotides A and G may represent “zeros” and nucleotides C, T and U represent “ones”.

In an embodiment a set of nucleotides {A, C, G, T} is mapped to any combination of a set of binary numbers {00, 01, 10, 11}. For instance, where {A, C, G, T} is mapped to {00, 01, 10, 11} respectively, the string of nucleotides GATTACA would encode the binary string 10001111000100.

In an embodiment, a short string of binary digits encodes a byte and each byte corresponds to a symbol (also referred to as a “letter”) that can be used to construct a string of symbols to form a codeword. The symbols in the codeword may include alphanumeric and special characters such as j#n5@$$mc*&!m.

In another embodiment, nucleotides A, G, C, T and U are arranged in subsets of two or more nucleotides, wherein each subset represents a character or symbol. Thus, a word may be encoded into the nucleic acid sequence of a taggant by the suitable arrangement of nucleotide subsets. In an illustrative example, the subset “AGC” represents the letter T, subset “GCT” represents the letter A, subset “CTU” represents the letter G, and subset “ACC” represents the letter N. Thus, the word TAGGANT can be encoded into a taggant by the nucleic acid sequence “AGCGCTCTUCTUGCTACCAGC”.

In another embodiment, nucleotides A, G, C, T and U, individually or in subsets of two or more nucleotides, represents binary, ternary, quaternary, and so on, to n-ary bits in a symbol. In an illustrative example, for a quaternary encoding system, the subset “AGC” represents the number 0, subset “GCT” represents the number 1, subset “CTU” represents the number 2, and subset “ACC” represents the number 3. Thus, the number 1230 can be encoded into a taggant by the nucleic acid sequence “GCTCTUACCGCT”, the number 133102 can be encoded into a taggant by the nucleic acid sequence “GCTACCACCGCTAGCCTU”, and so on. In the case of quaternary code, a string of two or more quaternary digits, for example 133102, may comprise a “crumb”. A “crumb” in quaternary code is equivalent to a ‘byte’ in binary code. A crumb may encode any character or symbol (letter), so that a string of crumbs encodes a string of symbols in a codeword. The string of n-ary digits used to encode each byte, crumb etc. should be designed with a specified mutual minimum Hamming or Levenshtein distance as will be familiar to persons skilled in the art.

In an embodiment disclosed herein, the target nucleic acid sequence comprises nucleotides selected from the group consisting of adenine, thymine, guanine, cytosine and uracil and wherein the nucleic acid sequence is a binary code where each of the nucleotides represent a string of 1's or 0's of length≥lbp.

In an embodiment disclosed herein, the target nucleic acid sequence comprises a subset nucleotides selected from the group consisting of adenine, thymine, guanine, cytosine and uracil, wherein the subset encodes a character.

In an embodiment disclosed herein, the target nucleic acid codeword sequence is assembled from a string of subsequences that are of length 2 bp or more (that are equivalent to a binary byte). The subsequences encode alphanumeric or special character symbols (e.g., j#n5@$$mc*&!m) so that variable region of the taggant encodes a string of alphanumeric and/or special characters that form a codeword. The codeword can then used to lookup information associated with a product, item or object on a database.

It is to be understood that the information that can be encoded into a taggant is limited only by the size the taggant and the arrangement of nucleotides, or subset of nucleotides, as representative of a binary, ternary, quaternary, . . . , n-ary code. In some instances, introducing redundancy will be desirable in view of sequencing and synthesis errors. Therefore, building in redundancy and error detecting and correcting capabilities may be incorporated into encoding design to increase decoding reliability. In these cases each crumb contains data nucleotides that encode the symbol and parity nucleotides that give error detection and correction capabilities. For example, taggant codewords can be constructed from Hamming (8,4,4) encoded symbols that contain four data nucleotides and four parity nucleotides. Other illustrative examples of encoding systems that have built in redundancy and/or error detecting and correcting capabilities include: Huffman encoding, Reed-Solomon encoding, Levenshtein encoding, differential encoding, single parity check encoding, Goldman encoding and XOR encoding¹⁻⁸.

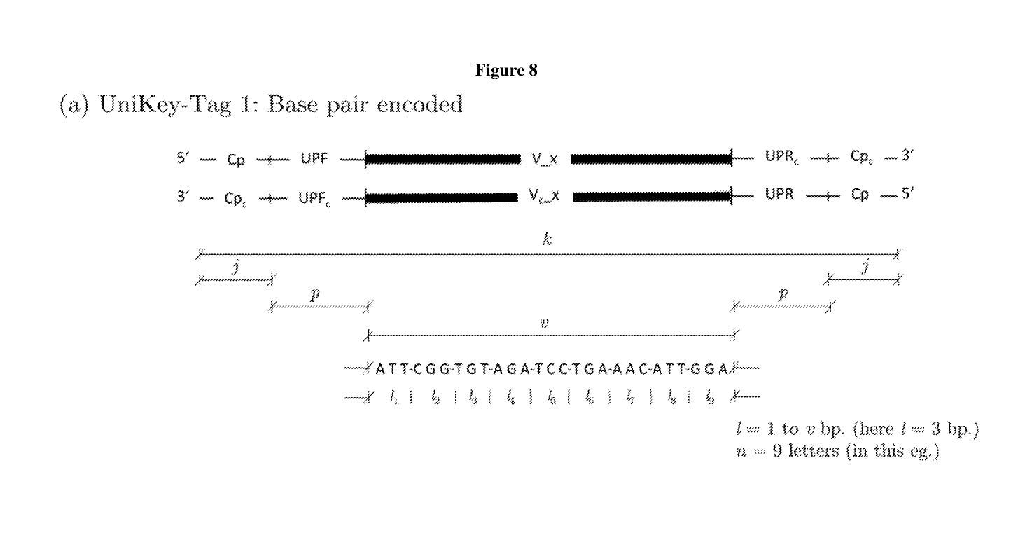

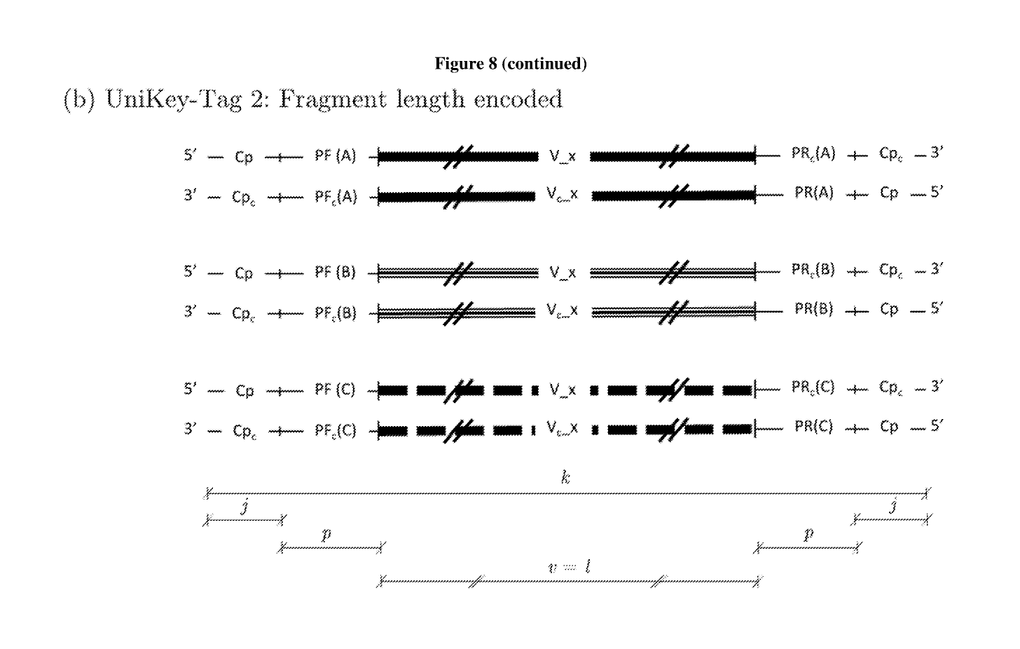

The string of nucleotides in the taggant may be subdivided to include information such as, for example, the expiry date, manufacturer, manufacturing facility and batch number of each precursor of a pharmaceutical product. In the simplest form, direct encoding requires each nucleotide to encode a letter (see, for e.g. FIG. 8 (a) where l≥1 bp).

In an embodiment, the taggant is encoded with a unique identifying alphanumeric and/or special symbol code that points to product, object, or identification information stored in a database. For example, the taggant may be encoded with codeword symbols 134-12-145-8-255-89 which is used to look up information in a database that may include the manufacturer, product type, manufacturing facility, product batch number, manufacturing date, and expiry date, for example.

In an embodiment the information encoded into a taggant is indicative of the date of manufacture. For instance, a manufacture can apply to its product or products a proprietary taggant comprising a nucleic acid sequence that encodes information of the date of manufacture; for example, “11 Jun. 2016”. The date of manufacture of a product or products can then be ascertained by sampling the product or products in such a way as to obtain the taggant (e.g., via a swab) and performing the methods disclosed herein to amplify the taggant or taggants present in the sample(s), wherein the information encoded by the taggant or taggants is indicative of the date of manufacture.

In an embodiment disclosed herein, the information encoded into a taggant is indicative of origin. Such methods can therefore be used to trace a product or products to their place or origin. For instance, a manufacturer can apply to its product or products a proprietary taggant comprising a nucleic acid sequence that encodes a proprietary n-ary code corresponding to characters that is indicative of that manufacturer, such as the name of the manufacturer, the address of the manufacturer, and the like. The place or origin can then be ascertained by sampling the product or products in such a way as to obtain the taggant (e.g., via a swab) and performing the methods disclosed herein to amplify the proprietary taggant or taggants, wherein the presence of the taggant is indicative of the product originating from the manufacturer, whereas the absence of the proprietary taggant may be indicative of a counterfeit product.

The information encoded by the target nucleic acid sequence can be decoded using routine methods known to persons skilled in the art. As used herein, the terms “decode” or “decoding” mean the conversion of nucleic acid sequences into an understandable form (e.g., an n-length codeword comprised of alphanumeric and/or special character symbols).

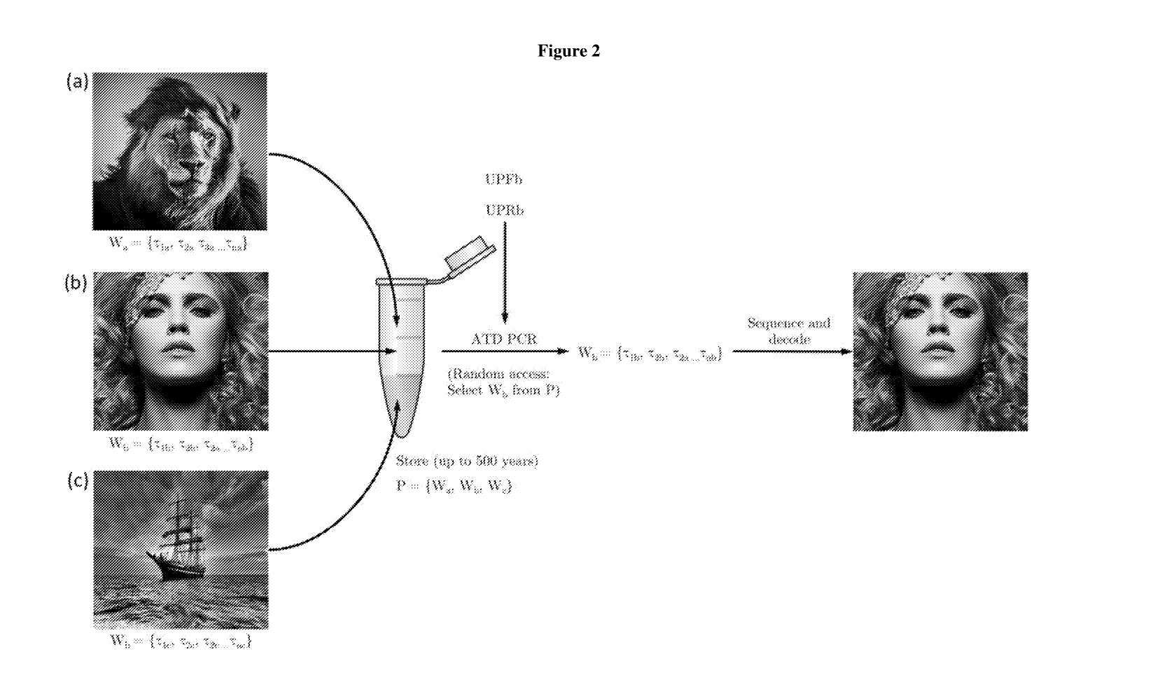

In an embodiment, the target sequence provides a means for archival data storage. As nucleic acids are inherently stable, molecular taggants are well suited to the archival storage of data, wherein the data are encoded by the arrangement of nucleotides or subset of nucleotides therein as representative of n-ary code that may be used to encode text, picture, or video files for example. Synthetic DNA sequences have been demonstrated to provide an effective means for the storage of data. For example, Bornholt et al. (2016) describes an architectural framework for a DNA-based archival storage system that is modeled as a key-value store. In an embodiment, DNA-based archival data is decoded by sequencing. For example, FIG. 2 shows three image files that are encoded by a specific library of fragments. Each library is defined by a specific set of forward and reverse primer sites that are universal to the file (e.g. UPFb, UPRb). The files are archived as a mixed pool of DNA fragments (P) comprising data for all three pictures encoded within the target sequences.

The methods disclosed herein also allow random access of a particular file in a single amplification reaction, while minimizing or otherwise avoiding fragment-fragment cross-fragment hybridization that can disrupt the decoding of DNA-based archival data. The amplification products produced by the methods disclosed herein may subsequently be sequenced and the image decoded from the resulting sequence. Files may also be divided into smaller library sets to allow for greater random access capability to, for example, access a particular part of a file.

As noted elsewhere herein, the information that can be encoded into a taggant is limited only by the size the taggant and the arrangement of nucleotides, or subset of nucleotides, as representative of binary, ternary, quaternary, . . . , or n-ary code.

For primer site encoded systems where samples are decoded by fragment length separation and/or the presence of PCR product and not sequencing (UniKey-Tag 2), an advantage of using LNA-containing primers is that the encoding letter length within a taggant can be compressed to a length 1 of about 10-15 bp without sacrificing binding affinity. LNA primers can therefore alter the design of the taggant. This reduction in 1 allows for an inversely proportional increase in the word sting length n according to Equation 1, below, which has significant implications for information storage capacity and taggant library size. Considering an alphabet of size s=5, if n is doubled from 5 to 10 the number of unique taggants available is increased from w₅=3,125 to w₁₀≈9.8 million according to Equation 3. LNA-primers may also be used to more efficiently decode primer pair based systems through the use of a universal forward primer and hierarchical sets of reverse primers to encode each L in S.

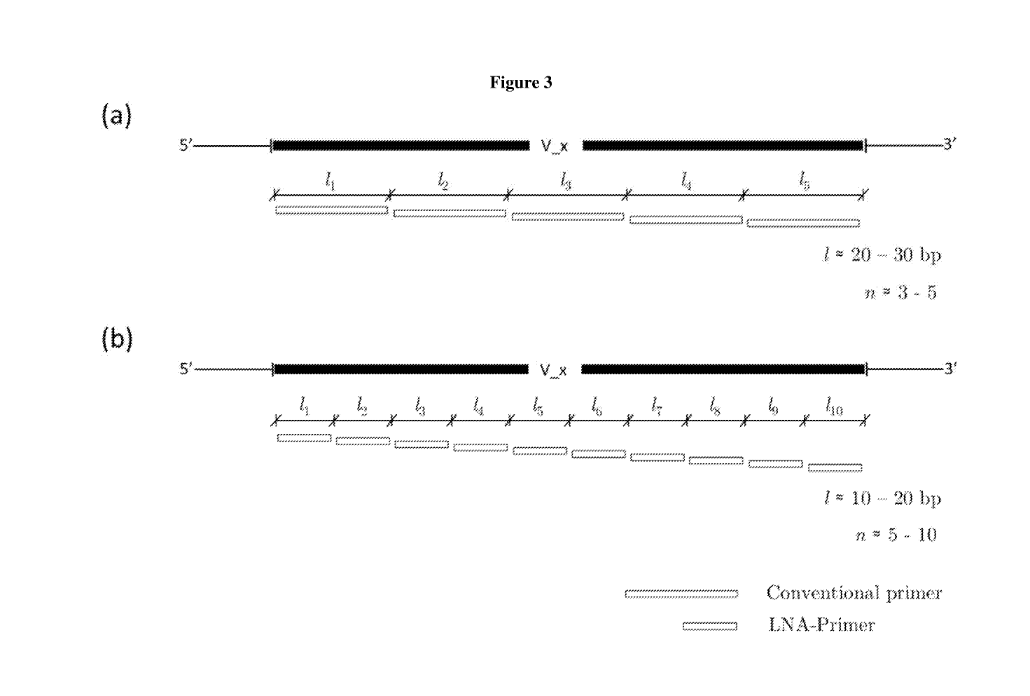

Encoding unit length compression also reduces the letter length 1 (bp) and thereby increases the codeword string length n of the taggants. For instance, the variable region of a taggant may be comprised of a string of nucleotides or nucleotide subsets that encode a letter from the set of characters in the alphabet, S. Watson-Crick DNA biochemistry dictates that l=about 20-30 bp for conventional nucleic acid primers to bind, and at the same time, oligonucleotide synthesis restrictions may, in some embodiments, limit the total fragment length to about k=100 bp. FIG. 3 (a) shows that this restricts the maximum coding string length to n=v/l=100/20=5 letters (where v=k), which, in turn, severely limits the size of the taggant library available wₙ according to the equation wₙ=sⁿ (see also Equation 3, below). For current taggant encoding technologies, wₙ is also restricted by s due to the number of amplification reactions required to screen Wₙ, which has flow-on implications for cost and low-copy number sampling. For example, the taggant library size of an n5-s5 system is w₅=5⁵=3,125 and the number of amplification screening reactions required for identification purposes is 250.

### Equation 1

The length of the coding string n is a function of the variable region length v bp and the letter length l bp:

$$n = \frac{v}{l}$$

### Equation 2

The size of the set of all codewords (such as a word string) w over the set of all symbols S is a function of the string length n and the size of the set of symbols s:

$${w}_{*} = {\sum }_{n = 1}^{n = {n}_{m ax}} {s}^{n}$$

### Equation 3

Where the taggant library size wₙ for a defined string length n is given as:

wₙ=sⁿ

As noted elsewhere herein, the target nucleic acid sequence (tag) comprises a first primer site and a second primer site. In some embodiments, the information encoded by the tag is found in the nucleic acid sequence between the first and second primer sites and therefore excludes the nucleic acid sequence of the first and second primer sites. In other examples, the information encoded by the tag can be found in the nucleic acid sequence that includes the first and/or second primer site. In one embodiment, the information is encoded by the nucleic acid sequence of the first primer site and the variable sequence. In another embodiment, the information is encoded by the nucleic acid sequence in the variable region and the second primer site. In yet another embodiment, the information is encoded by the nucleic acid sequence of the first primer site, the variable sequence and the second primer site.

### Tagging

The term “tagging”, as used herein, means the process of attaching, applying or otherwise incorporating a nucleic acid tag into or onto a product or article to allow for subsequent identification, authentication and/or tracing of the product by detection of the nucleic acid tag. The terms “product” and “article” are used interchangeably herein to denote a substance to which a nucleic acid tag can be applied, attached or otherwise incorporated. Nucleic acid tags can be applied to, attached to, or otherwise incorporated into, a product during the manufacture of said product or article. Alternatively, or in addition, nucleic acid tags can be applied to, attached to, or otherwise incorporated into, a product subsequent to its manufacture.

Suitable methods of tagging a product or article with a nucleic acid tag will be familiar to persons skilled in the art, illustrative examples of which are described in US 20050008762 to Sheu et al. In an embodiment, where the product is a liquid, a gas or an emulsion, the taggant can be distributed throughout the liquid, gaseous or emulsified medium by mere admixture. In another embodiment, where the product is a solid, the taggant can be applied in solution to the product or article and subsequently allowed to dry thereon.

Other illustrative examples of products or articles to which the nucleic acid taggants may be applied, attached or otherwise incorporated include plants and plant products (e.g., fruit, vegetables and grain), animals and animal products (e.g., meat, milk, cheese), explosives (e.g., plastic explosives and gunpowder), aerosols (e.g., automobile or industrial pollutants), organic solvents (e.g., from chemical processing plants), paper goods (e.g., newsprint, money, and legal documents), inks, perfumes, and pharmaceutical products or precursors thereof. For example, a precursor of a pharmaceutical product can be one component of a multi-component pharmaceutical composition. For instance, the precursor may be an active ingredient or an excipient. Therefore, where the pharmaceutical product comprises two or more active ingredients and/or two or more excipients, a different nucleic acid tag can be applied to each active ingredient or excipient prior to formulating the final pharmaceutical product.

As noted elsewhere herein, the product to which a nucleic acid tag can be applied, attached or other incorporated can be a solid, a liquid or a gas, whether inert or chemically active. Illustrative examples of inert solids include paper, pharmaceutical products or precursors thereof, wood, foodstuffs and polymer compounds (e.g., plastics).

In some embodiments, nucleic acid tags can be deposited (for example by spraying) onto the surface of a solid product. In other embodiments, the nucleic acid tags can be admixed with a liquid or gaseous product. For gases, the tag may be simply mixed with the gas. For example, containerized gases would have the tag placed in the container. For gases being released into the atmosphere, the tag could be mixed before release or at the time of release. For example, to track the pattern of dispersal of gases released by industry, one could attach an aerosol delivery device to an exhaust outlet and introduce a metered mount of tag as the gas is released. In another embodiment, nucleic acid tag or tags may be attached to a microparticle or nanoparticle and subsequently dispersed throughout the gaseous or liquid substance.

In some embodiments, the product may be exposed, or at risk of exposure, to conditions that may degrade the nucleic acid tag, such as nuclease activity, heat, pressure and UV light. It may therefore be advantageous to further provide a protective composition to the nucleic acid tags, either during application to the product or subsequent thereto. Suitable protective compositions will be familiar to persons skilled in the art, illustrative examples of which include encapsulating the nucleic acid tags (e.g., within liposomes, micelle bodies, silica) to protect them from enzymatic or chemical degradation, polymeric substances (e.g., proteins) and fixing agents. In an embodiment disclosed herein, the nucleic acid tag is applied with a solution comprises a fixing agent (e.g., an agent capable of fixing the tag to the product or article and/or protecting the taggant against adverse conditions such as high temperature, high pressure, UV light and nuclease activity). Suitable fixing agents will be familiar to persons skilled in the art. In some embodiments, the fixing agent is selected from the group consisting of polyvinyl alcohol, D-(+)-trehalose dehydrate and α,β-trehalose.

### Amplification of Nucleic Acids.

As used herein, the phrase “high fidelity amplification” typically means the amplification of a target nucleic acid sequence while minimizing or avoiding amplification of products that may be formed, for example, by non-specific fragment-fragment cross-hybridization through target sequence heterodimer formation, wherein such products would otherwise impact the amplification and/or identification of target nucleic acid sequences. Non-specific hybrid amplification products are also referred to herein as “non-specific amplicons”. As used herein, “cross-hybridization” refers to the hybridization of target nucleic acid fragments with other target nucleic acid fragments during thermocycling, in particular during the annealing phase of thermocycling, resulting in hybrid fragments of mixed origin and length. Cross-hybridization is the result of fragment-fragment priming and strand elongation during amplification.

Cross-hybridization is particularly problematic when amplifying multiple taggants comprising different nucleic acid sequences, because of the potential for cross-hybridization of complementary strands across the different target sequences occurring at a similar annealing temperature as primer-fragment hybridization. Cross-hybridization is especially problematic during the amplification of multiple nucleic acids comprising different nucleic acid sequences with common forward and reverse primer sites. Where cross-hybridization of complementary strands occurs, the resulting fragments are subsequently amplified, producing amplification products of mixed origin and length that makes it difficult, if not impossible, to identify a target sequence. As noted elsewhere, the methods disclosed herein minimize or otherwise avoid fragment-fragment cross-hybridization by using forward and reverse primers, each comprising at least one locked nucleic acid (LNA). This enables the annealing phase of the thermocycling amplification reaction to be conducted at a higher temperature that allows for the formation of LNA primer-fragment hybridization, but discriminates against the formation of fragment-fragment complexes that would otherwise occur at lower annealing temperatures.

Cross-fragment hybridization typically occurs during amplification reactions when fragment-fragment interactions occur at the same or similar conditions as primer-fragment interactions. This presents major problems when amplifying target nucleic acids sequences with common forward and reverse primer sites (see FIG. 4) and/or fragments contain common subsequences that encode a symbol (see FIG. 5). The benefit of using common primers, as described elsewhere herein, is that a universal set of primer ‘keys’ dramatically reduces the number of samples and reactions required to screen a sample. Examples of the mechanisms by which cross-fragment hybridization can occur are described with reference to the fragment-priming diagrams in FIG. 4 and FIG. 5.

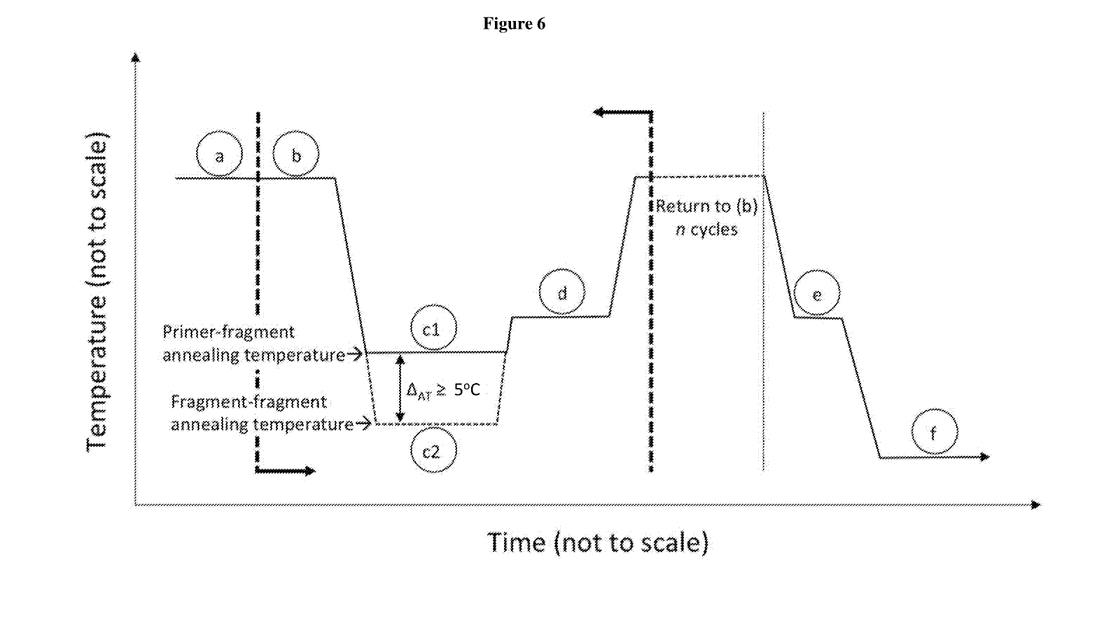

FIG. 4 shows a pool of oligonucleotide fragments that have different variable regions but the same forward and reverse primer sites. During the first step of PCR dsDNA fragments are denatured at high temperature (FIG. 6a) to form a mixture of ssDNA fragments with exposed base pairs (FIG. 4a). The reaction is then cooled to allow primers to bind to the exposed strands, providing a double stranded template for DNA polymerase mediated strand elongation. The temperature at which primers bind to the template is referred to as the annealing temperature (FIG. 6; c1 and c2). Cross-fragment hybridization between different oligonucleotides that share common primer sites occurs because interactions between these complementary sites occur at the same or similar annealing temperature conditions as primer-fragment interactions.

The mechanisms of cross-fragment hybridization are shown in FIG. 4 (b, iv). First (i) cross-fragment priming and elongation occurs between common primer sites, which (ii) results in the first generation of fragments with hybridized variable regions. Successive generations of cross-priming continues with each PCR thermal cycle, further ‘shuffling’ the variable regions. Non-specific binding between the hybrid fragments results in runaway priming and elongation (iii) and ultimately produces products of mixed origin and length (iv). In the majority of cases, cross-fragment hybridization makes it impossible to determine the sequence of the original fragments/taggants.

FIG. 5 shows the mechanism of cross fragment hybridization between common symbol sequences in different taggant codewords. Cross-symbol priming is a particular problem when the same symbol sequences (referred to ‘bytes’ in binary code and ‘crumbs’ in quaternary code) are used in a different codewords. Cross-symbol priming is likely to occur if the same encoding system is used to generate taggant codewords that are subsequently mixed together. Similarly, cross fragment hybridization is more likely to occur between variable regions that are GC-rich according to Watson Crick DNA binding biochemistry.

Suitable techniques for thermocycling amplification of target nucleic acid sequences are known to the person skilled in the art, illustrative examples of which include polymerase chain reaction (PCR), ligase chain reaction (LCR), gap filling LCR (GLCR), Qβ replicase, Strand Displacement Amplification (SDA), Self-Sustained Sequence Replication (3 SR), Nucleic Acid Sequence-Based Amplification (NASBA) and variations thereof.

In an embodiment, amplification is performed by PCR, illustrative examples of which are described in U.S. Pat. No. 4,683,195 and related U.S. Pat. Nos. 4,683,202; 4,800,159 and 4,965,188. In an embodiment, PCR is initiated by combining a sample suspected of comprising a target nucleic acid sequence (also referred to herein as a nucleic acid “template”), two primer sequences (forward and reverse), PCR buffer, free deoxynucleoside tri-phosphates (dNTPs) and thermostable DNA polymerase, such as Taq polymerase. Thereafter, the mixture is heated to separate, or “melt” the double-stranded DNA template, also referred to herein as the “melting phase”. A subsequent “annealing phase” allows the primers to anneal to complementary sequences on the single-stranded template or target sequence to be amplified. Replication of the target sequence occurs during the “extension phase”, whereby the DNA polymerase produces a strand of DNA that is complementary to the template. Repetition of this process doubles the number of copies of the sequence of interest, and multiple cycles increase the number of copies exponentially. In an embodiment, amplification comprises at least 10 cycles of melting, annealing and extension. In an embodiment, amplification comprises at least 20 cycles of melting, annealing and extension. In an embodiment, amplification comprises at least 30 cycles of melting, annealing, and extension. In an embodiment, amplification comprises at least 40 cycles of melting, annealing, and extension. In an embodiment, amplification comprises at least 50 cycles of melting, annealing, and extension. In an embodiment, amplification comprises between 10 and 50 cycles of melting, annealing and extension. In an embodiment, amplification comprises between 20 and 50 cycles of melting, annealing and extension. In an embodiment, amplification comprises between 30 and 50 cycles of melting, annealing and extension.

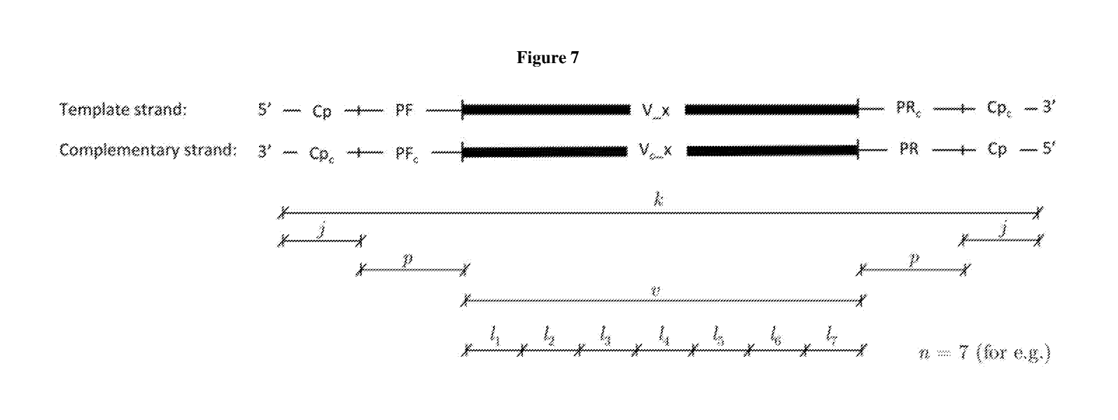

It is to be understood that the methods disclosed herein are not limited to the amplification of target nucleic acid sequences of a finite size. However, persons skilled in the art will recognize that amplification efficiency is dependent, at least in part, on the size of the target nucleic acid sequence. In an embodiment, the taggant is not more than 2000 base pairs (bp) in length. In an embodiment, the taggant is not more than 1000 base pairs (bp) in length. In an embodiment, the taggant is not more than 500 base pairs (bp) in length. In an embodiment, the taggant is not more than 300 base pairs (bp) in length. In an embodiment, the taggant is not more than 200 base pairs (bp) in length. In another embodiment, the taggant is not more than 100 base pairs (bp) in length. In an embodiment, the taggant is not more than 50 base pairs (bp) in length. An illustrative example of a taggant suitable for amplification in accordance with the present invention is provided in FIG. 7 and FIG. 8. The taggant suitable for use with the present invention suitably comprises a first primer site, a second primer site, and a variable region in between the first and second primer sites. In an embodiment, the taggant further comprises 5′ and 3′ capping regions.

### LNA Primers

As used herein the term “primer” means an oligonucleotide that is capable of annealing to another nucleic acid of interest under conditions suitable for amplification by thermocycling. The ability of a primer to anneal to a primer site is dependent, at least in part, on the degree of complementarity between the nucleotide sequence of the primer and the nucleotide sequence of the primer sites.

A primer is typically a short nucleic acid sequence of about 8 to about 60 bases, preferably of about 8 to about 30 nucleotides. In some embodiments, the primers are between 15 and 25 nucleotides in length. In some embodiments, the first and/or second primers (forward and/or reverse primers) may comprise additional nucleic acids at the 5′ end. This can be advantageous where the length of the target nucleic acid (tag) may otherwise be too small for detection (e.g., below the minimum read length limit of a sequencing protocol); hence, incorporating additional nucleic acids at the 5′ end of the forward and/or reverse primers can generate larger fragments suitable for subsequent detection. It is to be understood that, where it may be desirable to incorporate additional nucleic acids at the 5′ end of the forward and/or reverse primers, it is unnecessary to incorporate LNA into the extended portion (i.e., the 5′ tail) of the forward and/or reverse primer.

As used herein the terms “complementary” or “complementarity” mean nucleic acids (i.e. a sequence of nucleotides) related by the well-known base-pairing rules that A pairs with T or U and C pairs with G. For example, the sequence 5′-A-G-T-3′ is complementary to the sequence 3′-T-C-A-S′ in DNA and 3′-U-C-A-S′ in RNA. Complementarity can be “partial” in which only some of the nucleotide bases are matched according to the base pairing rules. On the other hand, there may be “complete” or “total” complementarity between the nucleic acid strands when all of the bases are matched according to base-pairing rules. The degree of complementarity between nucleic acid strands has significant effects on the efficiency and strength of hybridization between nucleic acid strands as known well in the art. This is of particular importance in embodiments where target sequences contain common primer sites and/or common symbol sequences in the variable encoding region of the taggant.

As used herein the terms “locked nucleic acid” or “LNA” means a nucleic acid analogue that contains a methylene bridge connecting the 2′-O and the 4′-C atom of the ribose monosaccharide.

As described herein, the inventor has shown that the incorporation of at least one LNA into the first and/or second primer allows the annealing temperature of an amplification reaction to be elevated to allow for the formation of LNA primer-fragment complexes, whilst discriminating against the formation of fragment-fragment complexes that would otherwise occur at lower annealing temperatures. As used herein, the terms “LNA primer” and “LNA-primer” refer a primer that comprises at least one LNA.

The thermocycling amplification reaction employed by the present invention is also referred to herein as “annealing temperature discrimination PCR” or “ATD PCR”. This method eliminates cross-fragment hybridization by (1) artificially elevating the annealing temperature of primer-fragment interactions and (2) setting the PCR annealing temperature to facilitate the formation primer-fragment complexes (see FIG. 6; c1) but discriminate against the formation fragment-fragment complexes that occur at a lower temperature (see FIG. 6; c2). To discriminate against cross-hybridization, the primer-fragment annealing temperature is elevated (e.g., by at least 5° C.) above the fragment-fragment annealing temperature (i.e., Δ_{AT} is at least 5° C.). This is achieved by incorporating at least one locked nucleic acid (LNA) monomer into the forward and/or reverse primers, preferably both the forward and reverse primers.

The elevated annealing temperature, therefore, reduces the affinity of interactions between complementary or near complementary sequences in the target fragments (ie. fragment-fragment interactions). Thus, the higher annealing temperature of ATD PCR can be used as a selective condition to allow the thermal cycle annealing temperature to be set sufficiently high to eliminate cross-fragment interactions between (1) common primer sequences and/or (2) common symbol sequences.

Thus, ATD PCR comprises the use of first and second primers that include at least one LNA and wherein the temperature of the annealing phase is elevated such that it allows for the formation of LNA primer-fragment complexes, but discriminates against the formation of fragment-fragment complexes that would otherwise occur at a lower annealing temperature. By elevating the annealing temperature of the thermocycling reaction, ATD PCR allows for the formation of LNA primer-fragment complexes whilst ensuring there is substantially no annealing of nucleic acid sequences that do not include at least one LNA (e.g., fragment-fragment complexes). In an embodiment, the temperature of the annealing phase is elevated such that there is substantially no annealing of nucleic acids between the first and second primer sites of the target nucleic acid sequences.

As used herein, the phrase “substantially no annealing” refers to a level of annealing that would be insufficient to produce an amplification product detectable by, for example, gel electrophoresis and labelling with ethidium bromide. Therefore, the phrase “substantially no annealing of nucleic acid sequences that do not include at least one LNA” means that at least 90%, at least 95%, or preferably at least 99% of detectable amplification products are the result of the annealing of nucleic acid sequences that include at least one LNA.

The number of LNA in each of the first and second primers should be such that it allows annealing of the primers to the corresponding primer sites of the target nucleic acid sequence at an elevated temperature at which there is substantially no annealing of nucleic acid sequences that do not include at least one LNA; that is, at a temperature that discriminates against fragment-fragment cross-hybridization.

In an embodiment disclosed herein, the number of LNA in the first or second primer is selected such that it allows the primers to hybridize to their respective primer sites during the annealing phase at a temperature that is at least 5° C. higher than the temperature at which the first and/or second primers would hybridize to their respective primer sites in the absence of an LNA; that is, at least 5° C. higher than the temperature at which nucleic acid sequences that do not include at least one LNA would anneal. In an embodiment, the annealing temperature is at least 6° C. higher, preferably at least 7° C. higher, preferably at least 8° C. higher, preferably at least 9° C. higher and more preferably at least 10° C. higher, or 5° C. to 10° C. higher, than the temperature at which the first and/or second primers would hybridize to their respective primer sites in the absence of an LNA; that is, higher than the temperature at which nucleic acid sequences that do not include at least one LNA would anneal.

Persons skilled in the art will understand that the optimum or near optimum annealing temperature of a thermocyclic amplification reaction such as PCR will largely depend on the length and composition of the primers. In an embodiment, the temperature used during the annealing phase is between about 50° C. and 72° C. In another embodiment, the temperature used during the annealing phase is between about 65° C. and 72° C. In another embodiment, the temperature used during the annealing phase is between about 67° C. and 72° C. In another embodiment, the temperature used during the annealing phase is between about 67° C. and 69° C.

In an embodiment disclosed herein, the first and/or second primers each comprise between 1 to 14 LNA. In an embodiment, the first primer comprises between 1 and 8 LNA. In an embodiment, the first primer comprises between 2 and 10 LNA. In an embodiment, the first primer comprises between 2 and 8 LNA. In a preferred embodiment, the first primer comprises between 3 and 7 LNA. In an embodiment, the first primer comprises at least 1 LNA, at least 2 LNA, at least 3 LNA, at least 4 LNA, at least 5 LNA, at least 6 LNA, at least 7 LNA, at least 8 LNA, at least 9 LNA, at least 10 LNA, at least 11 LNA, at least 12 LNA, at least 13 LNA, or at least 14 LNA. In an embodiment, the second primer comprises between 1 and 8 LNA. In an embodiment, the second primer comprises between 2 and 10 LNA. In an embodiment, the second primer comprises between 2 and 8 LNA. In a preferred embodiment, the second primer comprises between 3 and 7 LNA. In an embodiment, the second primer comprises at least 1 LNA, at least 2 LNA, at least 3 LNA, at least 4 LNA, at least 5 LNA, at least 6 LNA, at least 7 LNA, at least 8 LNA, at least 9 LNA, at least 10 LNA, at least 11 LNA, at least 12 LNA, at least 13 LNA, or at least 14 LNA.

In an embodiment, the first and second primers comprise the same number of LNA. It is to be understood, however, that there is no requirement that the first and second primers comprise the same number of LNA and that the methods disclosed herein can be performed where the first and second primers comprise a different number of LNA. As illustrative examples, the first primer comprises 1 LNA and the second primer comprises 2 LNA, the first primer comprises 2 LNA and the second primer comprises 1 LNA, the first primer comprises 1 LNA and the second primer comprises 3 LNA, the first primer comprises 3 LNA and the second primer comprises 1 LNA, the first primer comprises 3 LNA and the second primer comprises 2 LNA, the first primer comprises 4 LNA and the second primer comprises 1 LNA, and so on.

LNAs may be incorporated into the first and second primers at any suitable location. In an embodiment, the first primer and/or second primer comprises at least one adjacent pair of LNA. In an embodiment at least one of the adjacent pair of LNA is an adenine (A) or a thymine (T).

The incorporation of at least one LNA into the first primer and second primer has the additional advantage of reducing the length of the primer required to specifically anneal to the first and second primer sites, respectively. Conventional nucleic acid primers are generally restricted to between 20-30 bp due to biochemical limitations. However, LNA-primers may be reduced to between 5 and 15 nucleotides in length without a substantial reduction in their ability to hybridize (i.e., anneal) to the complementary strands of the first and second primer sires.

In an embodiment, the first and second primers each comprise between 5 and 30 nucleotides. In another embodiment, the first and second primers each comprise between 8 and 20 nucleotides. In another embodiment, the first and second primers each comprise between 5 and 10 nucleotides. In an embodiment, the first and/or second primer comprise a nucleic acid sequence selected from SEQ ID NOs: 61 to 68.

### Taggant Layering

As noted elsewhere herein, the present invention is particularly suited to taggant layering; that is, to the identification of multiple target nucleic acid sequences in a mixture thereof, as it avoids or minimizes the probability of fragment-fragment cross-hybridization between different target sequences during the annealing phase of thermocycling amplification. This is of particular importance to the invention disclosed herein, which relates to the amplification of target nucleic acid sequences that have common primer sequences that flank a variable region that may contain common symbol sequences in the codeword.

Thus, in another aspect disclosed herein, there is provided a method for high fidelity amplification of two or more target nucleic acid sequences in a mixture thereof, wherein each of the two or more target nucleic acid sequences are flanked by a first primer site and a second primer site, wherein the amplification comprises thermocycling involving a melting phase, an annealing phase and an extension phase, the method comprising using a first primer complementary to each of the first primer sites and a second primer complementary to each of the second primer sites, wherein each of the first and second primers comprise at least one locked nucleic acid (LNA) and wherein an elevated temperature is used during the annealing phase of the thermocycling such that, during the annealing phase, there is substantially no annealing of nucleic acid sequences other than of the first and second primers to the first and second primer sites, respectively.

In another aspect disclosed herein, there is provided A method for high fidelity amplification of two or more target nucleic acid sequences in a mixture thereof, wherein each of the two or more target nucleic acid sequences are flanked by a first primer site and a second primer site, wherein the amplification comprises thermocycling comprising a melting phase, an annealing phase and an extension phase, the method comprising using a first primer complementary to each of the first primer sites and a second primer complementary to each of the second primer sites, wherein each of the first and second primers comprise at least one locked nucleic acid (LNA) and wherein an elevated temperature is used during the annealing phase of the thermocyling such that, during the annealing phase, there is substantially no annealing of nucleic acid sequences other than of the first and second primers to the first and second primer sites, respectively,

wherein one or more or all of the following apply,
i) the two or more target nucleic acid sequences are amplified in a single thermocycling reaction;
ii) the two or more target nucleic acid sequences encode non-biological information; or
iii) each of the two or more target nucleic acids are flanked by a common first primer site and a common second primer site.

In an embodiment, the methods described herein further comprise high fidelity amplification of an additional two or more target nucleic acid sequences flanked by a third primer site and a fourth primer site, which are different to the first and second primer site.

The term “taggant layering” is used herein to denote the process of intentionally marking elements of matter (i.e. product precursors) with different taggants so that the authenticity or identity of each element can be established from the combined matter. For example, the precursors of a pharmaceutical product may be marked with a taggant that identifies the origin, date of production, manufacturer or other relevant information of each precursor. Identification, as opposed to authentication, aims to establish the origin of unknown matter. Authentication aims to validate a hypothesis that unknown matter is of a particular origin and only gives a yes or no outcome. For example, authentication asks the question ‘Is this product X?’, and gives the ‘yes’ or ‘no’. Identification asks the question, ‘What product is this?’ and gives the answer ‘this is product X, Y, and/or Z’.

For example, ammunition may be marked so that a taggant signature is left on the user, gun, casing, bullet entry point and bullet. Without prior knowledge of the taggant(s) present on the bullet, the entire library of millions or billions of possible candidate taggants (i.e. for a country, region or the world) may be screened simultaneously using the amplification methods disclosed herein to identify the subset of taggants present. Both taggant layering and identification require the capacity to screen and decode a subset of unknown taggants from a library of billions of taggants.

As used herein, the term “layering depth” means the size of a subset of taggants in a defined taggant library that may be mixed and decoded.

As used herein the term “deep layering” means the marking of more than 100 elements of matter that may be mixed and decoded.

Where the methods disclosed herein are employed for taggant layering and identification (i.e., the amplification of two or more target nucleic acid sequences), it is to be understood that each taggant may comprise its own set of first and second (forward and reverse) primer sites. Thus, in an embodiment, the first of the two or more target sequences is flanked by a first primer site and a second primer site, the second of the two or more target sequences is flanked by a third primer site and a fourth primer site, and so on.

However, since the methods disclosed herein provide amplification of target nucleic acid sequences while minimizing, or otherwise avoiding, cross-fragment hybridization, a universal set of primers (also referred to herein as primer ‘keys’) can be used to ‘unlock’ millions of fragments in one amplification reaction. Advantages over the prior art include orders of magnitude improvements in information storage capacity, information decoding efficiency and taggant layering capacity. The methods disclosed herein also allow an unknown subset of taggants to be identified from a pool of billions of taggants.

It is also to be understood that at least two of the two or more target nucleic acid sequences may share a common forward or reverse primer site. As an illustrative example, the first of the two or more target sequences are flanked by a first primer site and a second primer site and the second of the two or more target sequences is flanked by the first primer site of the first target sequence and a third primer site.

In an embodiment disclosed herein, the two or more target nucleic acid sequences are flanked by a common first primer site and a common second primer site. The term “common” is used interchangeably herein with the term “universal” to mean that the first primer sites and the second primer sites across two or more target sequences have the same or substantially the same nucleic acid sequence. Ideally, the sequence of the first primer site (e.g. forward primer site) of a first target nucleic acid sequence is identical to the sequence of the first primer site of a second target nucleic acid sequence. It is to be understood, however, that the methods disclosed herein can also be performed where the primer sites are not completely (i.e., 100%) identical, but rather substantially identical or substantially the same. The terms “substantially the same” and “substantially identical” mean that the sequence of the first primer site (e.g. forward primer site) of a first target nucleic acid sequence differs from the sequence of the first primer site of a second target nucleic acid sequence by 1 or more bases (e.g., by 1 base, by 2 bases, by 3 bases, by 4 bases, etc), while still retaining a degree of complementarity that would allow a primer to hybridize to the first primer sites of the first and second target nucleic acid sequence during the annealing phase of the thermocycling reaction.

Similarly, the terms “substantially the same” and “substantially identical” mean that the sequence of the second primer site (e.g. reverse primer site) of a first target nucleic acid sequence differ from the sequence of the second primer site of a second target nucleic acid sequence by 1 or more nucleic acids (e.g., by 1 base, by 2 bases, by 3 bases, by 4 bases, etc), while still retaining a degree of complementarity that would allow a primer to hybridize to the second primer sites of the first and second target nucleic acid sequence during the annealing phase of the thermocycling reaction.

In an embodiment, the two or more target nucleic acids are flanked by a common first primer site. In an embodiment, the two or more target nucleic acids are flanked by a common second primer site. In a preferred embodiment, the two or more target nucleic acids are flanked by a common first primer site and a common second primer site.

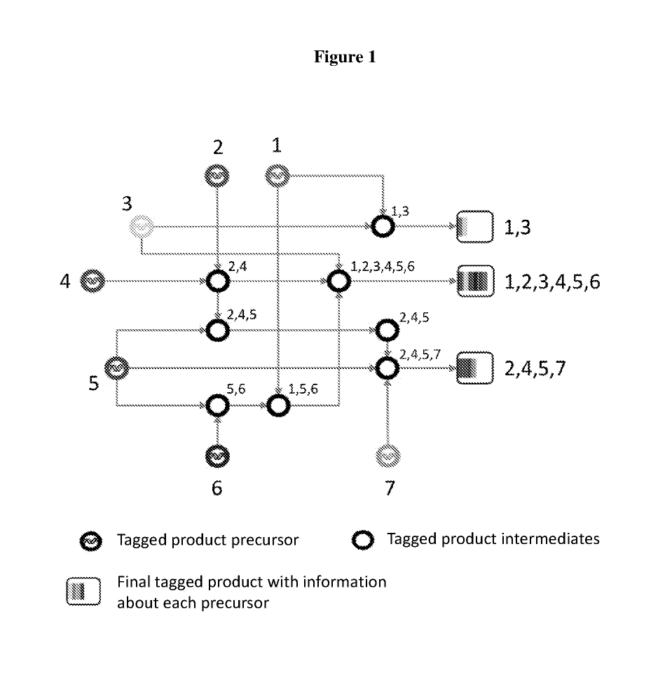

The use of common first and second primer sites allows for the amplification of multiple taggants in a single thermocycling reaction. Thus, in an embodiment, the two or more target nucleic acid sequences are amplified in a single thermocycling reaction. In an embodiment, no more than one first primer and one second primer are used. Accordingly, amplification of the nucleic acids can be achieved in a single step, without the need for additional primers, reagents, or thermocycling conditions. This is particularly useful for deep layering applications, for example, supply chain tracing in the pharmaceuticals or cosmetics industries. The capacity to screen billions of taggants simultaneously allows tagged product precursors to be mixed and decoded from the final product in a single reaction. A diagram of taggant layering/mixing is shown in FIG. 1. Although FIG. 1 shows seven tagged product precursors that are combined into a final product, there are no practical restrictions on the number of taggants that can be layered/mixed in the present invention. In an embodiment, a manufacturer may use a single set of common primer sites to define a class or batch of pharmaceutical products. Alternatively, it may be possible to use multiple sets of common primer sites to define individual precursors that may be used across different pharmaceutical products.

By using common primer sites, the methods disclosed herein solve the duel problems of taggant layering and identification by using one universal pair of primer ‘keys’ for each taggant library. This is achieved through the development of a novel amplification protocol that discriminates against fragment-fragment interactions, and designing taggants that exploit the full capabilities of sequencing by synthesis and nanopore technologies. The advantages of using common primer sites and one universal set of primer ‘keys’ (also referred to herein as the UniKey-Tag system) over existing technologies include orders of magnitude improvements in the size of the taggant library available, layering capacity, and efficiency with which the library is screened (in terms of number of reactions). The UniKey-Tag 1 system, for example, requires only one reaction to screen a library of billions of taggants whereas the current state-of-the-art requires several hundred reactions to screen a library of only thousands (U.S. Pat. No. 8,735,327).

The capacity of the UniKey-Tag system to trace and identify matter of mixed and uncertain origin (in the billions) opens a wide range of new applications including, for example, the tracing of illegal and counterfeit goods, pharmaceutical precursors, bank notes, cosmetics, electrical goods, food ingredients and clothing. As described elsewhere herein, UniKey-Taggants were successfully demonstrated to mark ammunition, such that a traceable chemical signature was recoverable from the user, gun, spent cartridge cases, bullet entry point and bullet after firing. For this application, the technology presents clear benefits for tracing illegal and black market arms transfers, detecting arms embargo violations, exposing weaknesses in stockpile management, tracing 3D-printed and modular weapons, identifying groups involved in the illegal wildlife trade, increasing forensic capabilities, and as a deterrent to gun crime.

The aim of taggant layering is to identify an unknown subset of taggants from an entire library of taggants. Increasing the layering depth could expand the range of applications to include, for example, product precursor tracing and regulated or black market goods identification. In an embodiment, the two or more target nucleic acid sequences are recovered from a pharmaceutical product or precursor thereof. In an embodiment, the two or more target nucleic acid sequences are recovered from a product selected from the group consisting of a firearm, ammunition, a projectile, firearm residue and a surface that has come into contact with a firearm, ammunition and/or projectile to which the two or more target nucleic acid sequences are applied.

### Identification

In an embodiment, the methods disclosed herein further comprise a step of detecting or identifying the amplified target nucleic acid sequences. Suitable methods of detecting or identifying the amplified target nucleic acid sequences will be familiar to persons skilled in the art, illustrative examples of which include sequencing (UniKey-Tag 1) and fragment size discrimination (UniKey-Tag 2) which includes, for example, running the amplified product(s) through an agarose or polyacrylamide gel and labeling the amplicon(s) with a suitable detectable label, such as ethidium bromide.

Where the methods disclosed herein are employed to identify two or more target nucleic acid sequences, such as in a mixture thereof, it is to be understood that the identification step needs to be sufficient to discriminate between the two or more target sequences. For instance, each of the two or more target nucleic acid sequences may have a different nucleic acid sequence, allowing the amplified targets to be identified by sequencing. Thus, in an embodiment, the methods disclosed herein further comprising the step of identifying the amplified two or more target nucleic acid sequences by sequencing. Alternatively, or in addition, each of the two or more target nucleic acid sequences can have a different length, allowing the amplified targets to be identified by size discrimination. Thus, in an embodiment, each of the two or more target nucleic acid sequences have a different length. In an embodiment, the methods disclosed herein further comprise the step of identifying the amplified two or more target nucleic acid sequences by size separation.

The term “identification”, as used herein, typically means determining the identity of a target nucleic acid sequence following amplification of the sequence in accordance with the methods disclosed herein. This is to be contrasted with “authentication”, which typically means testing for the presence of a known taggant or group of known taggants, wherein the taggants comprise nucleic acid sequences that are known prior to screening and decoding.

Identification of the amplification products may be achieved by any suitable means known to persons skilled in the art. As an illustrative example, nucleotides containing a detectable label may be incorporated in an amplicon during the extension phase of the thermocycling reaction, such that the amplicons can then be detected based on the presence of the detectable label. Suitable detectable labels will be familiar to persons skilled in the art, illustrative examples of which include radioisotopes, fluorophores and biotin. In another embodiment, the target nucleic acid sequence is identified based on fragment size. For example, following amplification, the reaction mixture is subjected to agarose gel electrophoresis, optionally alongside nucleotide markers of known sizes (base pairs). The target amplicon, having a predetermined size based on nucleotide length, and the markers migrate through the agarose gel and are subsequently stained with a detectable reagent such as ethidium bromide. The presence of the target nucleic acid sequence is then verified by the presence of an amplicon having a size that corresponds to the length of the target nucleic acid sequence, as determining by comparison to the adjacent markers.

Alternatively, or in addition, the identity of the target nucleic acid sequence is determined by sequencing the amplicon(s) from the amplification reaction and verifying the presence of an amplicon that has the same sequence as the target sequence. Suitable means of sequencing amplicons will be familiar to persons skilled in the art, illustrative examples of which include Sanger sequencing, next generation “sequencing by synthesis” and nanopore sequencing.

Where the methods disclosed herein are used to amplify two or more target nucleic acid sequences, the two or more amplicons may be identified by sequencing and/or by size. In an embodiment, where the methods disclosed herein are used to amplify two or more target nucleic acid sequences, each target nucleic acid sequence has a different length. Thus, the presence of the two or more target sequences can be determined by size (e.g., agarose gel electrophoresis). In another embodiment, where the methods disclosed herein are used to amplify two or more target nucleic acid sequences, each target nucleic acid sequence has a different sequence, whether or not each of the target sequences has the same length. Thus, the presence of the two or more target sequences can be determined by size (e.g., agarose gel electrophoresis) or sequence.

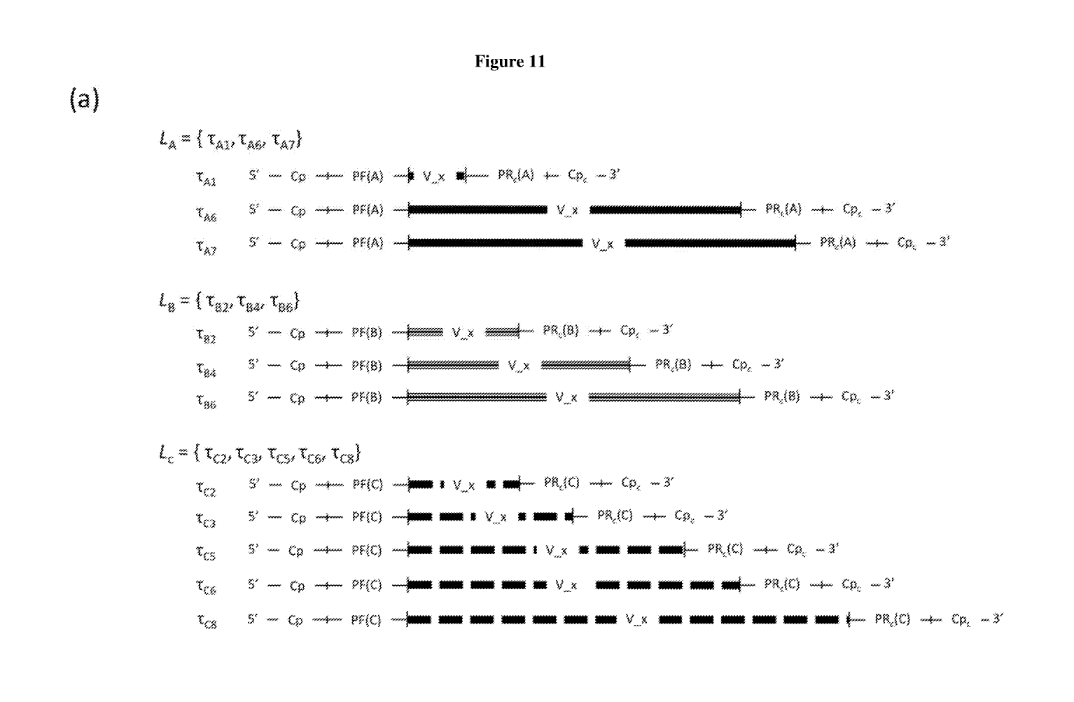

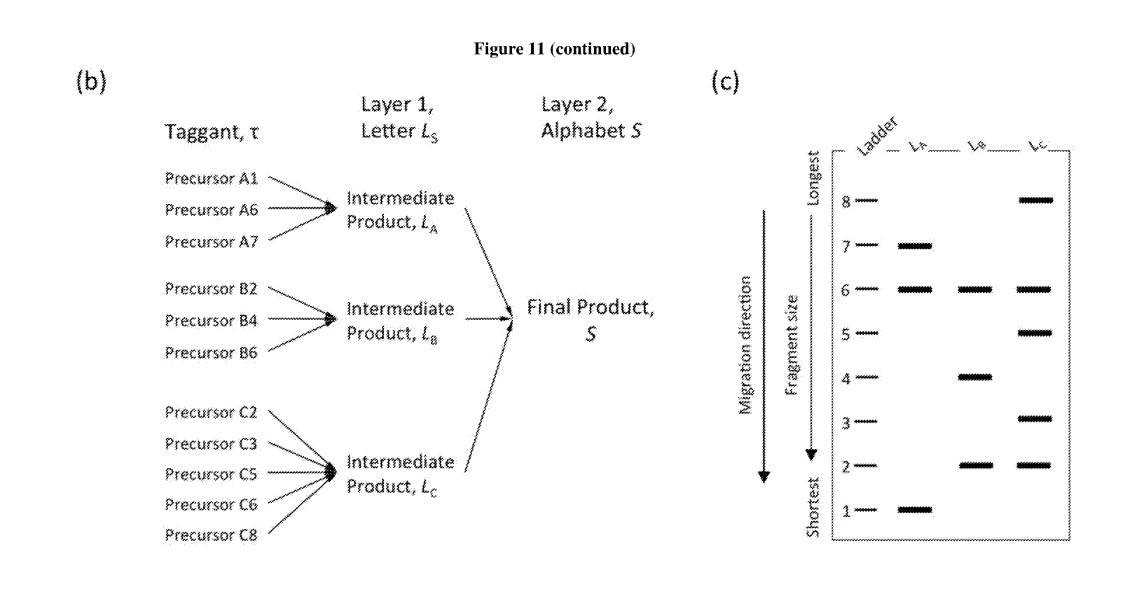

In the case of fragment identification by size (UniKey-Tag 2) the presence and length of a target sequence is indicative of origin (see, e.g., FIG. 11). For example, the presence of an ATD PCR product indicates the symbol type (for a particular primer pair) and the fragment lengths observed in a sample indicate the position of the symbol in the codeword string. Therefore, the number of amplification screening reactions required for the decoding of taggants according to target sequence length is equal to the size of the set of letters used, s (ie. number of symbols/‘letters’ in the alphabet). This is because each letter in the alphabet is identified with a unique set of primers that is amplified in a single reaction without cross-fragment hybridization. As such, each additional letter increases the layering depth in increments of up to 30, as defined by the fragment length separation resolution of polyacrylamide or agarose gels for fragments <100 bp, and requires only one additional screening reaction to decode.

### Taggant Libraries and Kits

As noted elsewhere herein, tagging of products using nucleic acid tags, as herein described, can be an effective means of identifying, authenticating, tracking and tracing products to which the taggants are applied, attached or otherwise incorporated. While nucleic acid tags can be attached, applied or otherwise incorporated into a product during its manufacture, it may be more convenient to attach, apply or otherwise incorporate nucleic acid tags into a product subsequent to its manufacture. The present disclosure therefore extends to a library of two or more nucleic acid tags that can be attached, applied or otherwise incorporated into a product during or subsequent to its manufacture. Accordingly, a single nucleic acid tag or multiple nucleic acid tags may be selected from the library to be applied or otherwise incorporated into the product.

Thus, in another aspect disclosed herein, there is provided a library of two or more nucleic acid tags, wherein each of the two or more nucleic acid tags is flanked by a common first primer site and a common second primer site. As noted elsewhere herein, the use of common first and second primer sites allows for the amplification of multiple taggants in a single thermocycling reaction. In an embodiment, each of the two or more nucleic acid tags has a different nucleic acid sequence, relative to the other tag(s) in the library.

In another aspect disclosed herein, there is provided a kit comprising a first component and a second component, wherein the first component comprises a library of two or more nucleic acid tags, wherein each of the two or more nucleic acid tags is flanked by a common first primer site and a common second primer site, and wherein the second component comprises a first primer complementary to the first primer site and a second primer complementary to the second primer site, and wherein the first and second primers each comprise at least one locked nucleic acid (LNA). In an embodiment, each of the two or more nucleic acid tags has a different nucleic acid sequence, relative to the other tag(s) in the library.

In an embodiment, the library comprises one or more nucleic acid sequences selected from SEQ ID NOs: 1 to 60.

In an embodiment, the two or more nucleic acid sequences encodes non-biological information, as herein described. In another embodiment, each of the first and second primers comprises between 1 and 14 LNA, as herein described. In an embodiment, each of the first and second primers comprises between 1 and 8 LNA. In an embodiment, each of the first and second primers comprises between 2 and 10 LNA. In an embodiment, each of the first and second primers comprises between 2 and 8 LNA. In a preferred embodiment, each of the first and second primers comprises between 3 and 7 LNA. In yet another embodiment, each of the first and second primers comprises at least one adjacent pair of adenine and thymine LNA.

In an embodiment, the first and/or second primer comprise a nucleic acid sequence selected from SEQ ID NOs: 61 to 68.

In an embodiment, the kit further comprises written instructions for the high fidelity amplification of the two or more nucleic acid tags in accordance with the methods described herein.

In another embodiment, the kit further comprises reagents for tagging a product with the library of two or more nucleic acid tags, which may include a fixing agent, as herein described.

In an embodiment, the kit further comprises a product selected from the group consisting of a firearm, ammunition and projectile to which the two or more target nucleic acid sequences are applied. In an embodiment, the kit further comprises a pharmaceutical product or precursor thereof to which the two or more target nucleic acid sequences are applied.

In another embodiment, the kit further comprises reagents for the high fidelity amplification of the two or more nucleic acid tags in accordance with the methods described herein, such as DNA (e.g., Taq) polymerase, buffers and nucleotide bases.

The first and second components of the kit are typically provided in separate containers or packaging. In some embodiments, however, one or more nucleic acid tags from the library are already applied, attached or otherwise incorporated into a product. For example, the library of two or more nucleic acid tags is applied, attached or otherwise incorporated into a product selected from the group consisting of a firearm, ammunition and a projectile.

### Tracing

As noted elsewhere herein, molecular tagging of products or articles using nucleic acid tags, as herein described, can be an effective means of identifying, authenticating, tracking and tracing products and articles to which the taggants are applied, attached or otherwise incorporated. Thus, in another aspect disclosed herein, there is provided a method of tracing a product to its origin, the method comprising:

(a) providing a product to which at least one nucleic acid sequence has been incorporated, wherein the at least one nucleic acid sequence is flanked by a first primer site and a second primer site;
(b) optionally recovering the at least one nucleic acid sequence from the product;
(c) amplifying the recovered at least one nucleic acid sequence by high fidelity amplification comprising thermocycling using a first primer complementary to the first primer site and a second primer complementary to the second primer site, wherein the first and second primers each comprise at least one locked nucleic acid (LNA), wherein the thermocycling comprises a melting phase, an annealing phase and an extension phase, and wherein an elevated temperature is used during the annealing phase of the thermocycling such that, during the annealing phase, there is substantially no annealing of nucleic acid sequences other than of the first and second primers to the first and second primer sites, respectively; and
(d) identifying the at least one nucleic acid sequence amplified in step (c);
wherein the sequence and/or length of at least one nucleic acid sequence identified in step (d) is indicative of the origin of the product.

In an embodiment, the product is selected from the group consisting of a firearm, ammunition, a projectile and firearm residue. In an embodiment, the product is a pharmaceutical product or precursor thereof. In an embodiment, the product is a cosmetic product or precursor thereof.

In an embodiment, the at least one nucleic acid sequence is recovered from the product. Thus, in an embodiment, step (b) is performed.

In an embodiment, the temperature used during the annealing phase of step (c) is such that there is substantially no annealing of nucleic acid sequences that do not include at least one LNA. In another embodiment, the temperature used during the annealing phase of step (c) is at least 5° C. higher than the temperature at which nucleic acid sequences other than the first and second primers would anneal. In an embodiment, the temperature used during the annealing phase of step (c) is at least 10° C. higher than the temperature at which nucleic acid sequences other than the first and second primers would anneal. In an embodiment, the temperature used during the annealing phase is between about 50° C. and 72° C. In another embodiment, the temperature used during the annealing phase is between about 67° C. and 72° C.

In an embodiment, each of the first and second primers comprises between 1 and 8, or 1 and 14, LNA. In a preferred embodiment, the first and second primers comprise between 3 and 7 LNA. In an embodiment, each of the first and second primers comprises at least one adjacent pair of LNA. In an embodiment at least one of the adjacent pair of LNA is an adenine (A) or a thymine (T).

In an embodiment, the method comprises recovering, amplifying and identifying two or more nucleic acid sequences. In an embodiment, each of the two or more nucleic acid sequences is flanked by a common first primer site. In an embodiment, each of the two or more nucleic acid sequences is flanked by a common second primer site. In an embodiment, each of the two or more oligonucleotide taggants has a different nucleic acid sequence. In an embodiment, step (d) comprises identifying the amplified two or more nucleic acid sequences by sequencing.

In another embodiment, each of the two or more nucleic acid sequences has a different length. In an embodiment, step (d) comprises identifying the amplified two or more nucleic acid sequences by size separation. In an embodiment, each of the two or more nucleic acid sequences encodes non-biological information.

As noted elsewhere herein, the methods and nucleic acid tags disclosed herein can be used to trace illegal firearms, detect arms embargo violations, expose weaknesses in stockpile management, trace 3D printed and modular weapons and identify groups involved in the illegal wildlife trade. For instance, a nucleic acid tag may be applied, attached or otherwise incorporated onto the surface of ammunition cartridges to provide an unbroken chain of identification linking the tag to a user, a gun, a cartridge case, bullet and/or a bullet entry point. An illustrative example is given in the Examples disclosed herein. For instance, one or more nucleic acid tags may be applied to ammunition or firearms so that a taggant signature is left, for example, on the user, gun, casing, bullet (projectile), firearm residue and/or a projectile entry point. Without prior knowledge of the tag(s) present on the bullet, the entire library of possible candidate tags may be screened to identify the tag or subset of tags present. Where common primer sites are used, the entire library of possible candidate taggants may be screened simultaneously with a common set of forward and reserve primers to identify the tag or subset of tags present, in accordance with the methods disclosed herein.

Thus, in an embodiment of the methods disclosed herein, the target nucleic acid sequence is recovered from a product selected from the group consisting of a firearm, ammunition, a projectile, firearm residue and a surface that has come into contact with a firearm, ammunition and/or projectile to which the target nucleic acid sequence is applied. In another embodiment, the target nucleic acid sequence is recovered from a surface of an entry point of a projectile fired from a firearm.

The taggant identification and decoding systems disclosed herein offer orders of magnitude improvements over existing technologies in terms of library size, recovery efficiency and layering depth. In comparison to existing taggant identification and decoding systems, the methods disclosed herein offer significant advantages in one or more of the following areas:
Scope, taggant library size: billions (unlimited) vs thousands;
Scope, taggant layering capacity: millions vs approx. twenty;
Efficient, decoding reactions required: one vs approx. three hundred;
Further efficiency improvements are not possible using synthetic DNA taggants: one reaction to decode, information encoded in nucleotide sequence which is the smallest indivisible unit of DNA.
Exploits the rapid learning curve of next generation sequencing technologies, which has far exceeded Moore's Law over the past decade.
Applications: Identification, deep layering, authentication. Capacity to trace materials of mixed and uncertain origin
Novel deep layering applications: Supply chain monitoring (tracing), pharmaceutical precursor tracing, ammunition tracing, counterfeit goods identification.

Those skilled in the art will appreciate that the invention described herein is susceptible to variations and modifications other than those specifically described. It is also to be understood that the invention includes all such variations and modifications that fall within the spirit and scope. The invention also includes all of the steps, features, compositions and compounds referred to or indicated in this specification, individually or collectively, and any or all combinations of any two or more of said steps or features.

Unless defined otherwise, all technical and scientific terms used herein have the same meanings as commonly understood by one of ordinary skill in the art to which this invention belongs.

Aspects of certain embodiments of the invention are further described by reference to the following non-limiting examples.

### EXAMPLES

### Example 1—Molecular Taggant Design and Preparation for Series 1 Experiments (9 mm Handgun)

For the Series 1 experiments described herein, single stranded DNA oligonucleotides (ssDNA) were synthesised by Sigma-Aldrich and purified using high-performance liquid chromatography (HPLC). Production was distributed across several different locations and over several weeks to ensure against cross contamination at the manufacturing facility. ssDNA oligonucleotides were designed with 5′ and 3′ end capping regions (3 bp), universal forward and reverse primer sites (20 bp), and codeword regions of variable length (14, 20, 24, 28, 32 bp).

In total, 40 complementary ssDNA oligonucleotides were ordered and subsequently annealed to form 20 dsDNA taggant duplexes (OligoTag_1_Ser1 to OligoTag_20_Ser1 in Table 1). These 20 taggants included four of each of the following lengths: 80, 76, 70, 66 and 60 bp so that identification could be performed by fragment length separation (UniKey-Tag 2). The taggant library used in Series 1 experiments was designed so that the variable region of each taggant (OligoTag_1_Ser1 to OligoTag_20_Ser1 in Table 1) was separated by a mutual distance of at least 50% of the length of the variable region from all of the other taggants in the library.

The design and specifications of the taggants used in Series 1 experiments are shown in Table 1. The nomenclature used herein is OligoTag_1(tag 1)T/C(template/complementary strand, respectively) Ser1(experiment series). In Table 1, the capping regions are in italics, the primer sites are shown in standard text, and the codeword sequences are in bold and enclosed by square brackets.

TABLE 1
Fragment sequences and specifications used in Series 1 experiments (9 mm
handgun ammunition fingerprinting).

| OligoTag_1_Series1 | | | | |
|---|---|---|---|---|
| OligoTag_1T_Ser1 | ATC-ATCTGACTTTCGTATCTAGC- | | | |
| (5′ → 3′) | [AGGATGCTGTGCAAGGAGAAGCAGCTACGTGTCA]- | | | |
| SEQ ID NO: 1 | CCATGAGAAGTTCATACACA-TAT | | | |
| OligoTag_1C_Ser1 | ATA-TGTGTATGAACTTCTCATGG- | | | |
| (5′ → 3′) | [TGACACGTAGCTGCTTCTCCTTGCACAGCATCCT]- | | | |
| SEQ ID NO: 2 | GCTAGATACGAAAGTCAGAT-GAT | | | |
| | Ds oligo: | | Ss oligo: | |
| | Length (bp) | 80 | Hairpin Tm (° C.) | 41.7 |
| | GC (%) | 43.8 | Hairpin deltaG (kcal mol⁻¹) | −5.95 |
| | Tm (° C.) | 88.5 | Self dimer (kcal mol⁻¹) | −7.05 |
| | deltaG (kcal mol⁻¹) | −142.8 | | |
| | Molecular mass | 49,303 | | |
| OligoTag_2_Series1 | | | | |
| OligoTag_2T_Ser1 | ATC-ATCTGACTTTCGTATCTAGC- | | | |
| (5′ → 3′) | [ACCTATCGACTCGTGTACGTGGATTCGTTCTACA]- | | | |
| SEQ ID NO: 3 | CCATGAGAAGTTCATACACA-TAT | | | |
| OligoTag_2C_Ser1 | ATA-TGTGTATGAACTTCTCATGG- | | | |
| (5′ → 3′) | [TGTAGAACGAATCCACGTACACGAGTCGATAGGT]- | | | |
| SEQ ID NO: 4 | GCTAGATACGAAAGTCAGAT-GAT | | | |
| | Ds oligo: | | Ss oligo: | |
| | Length (bp) | 80 | Hairpin Tm (° C.) | 37.2 |
| | GC (%) | 41.2 | Hairpin deltaG (kcal mol⁻¹) | −2.91 |
| | Tm (° C.) | 86.1 | Self dimer (kcal mol⁻¹) | −6.76 |
| | deltaG (kcal mol⁻¹) | −138.8 | | |
| | Molecular mass | 49,301 | | |
| OligoTag_3_Series1 | | | | |
| OligoTag_3T_Ser1 | ATC-ATCTGACTTTCGTATCTAGC- | | | |
| (5′ → 3′) | [ACAGTCGATCCTTCGTACCTGCTTCGATGGCAAT]- | | | |
| SEQ ID NO: 5 | CCATGAGAAGTTCATACACA-TAT | | | |
| OligoTag_3C_Ser1 | ATA-TGTGTATGAACTTCTCATGG- | | | |
| (5′ → 3′) | [ATTGCCATCGAAGCAGGTACGAAGGATCGACTGT]- | | | |
| SEQ ID NO: 6 | GCTAGATACGAAAGTCAGAT-GAT | | | |
| | Ds oligo: | | Ss oligo: | |
| | Length (bp) | 80 | Hairpin Tm (° C.) | 41.8 |
| | GC (%) | 42.5 | Hairpin deltaG (kcal mol⁻¹) | −5.11 |
| | Tm (° C.) | 88.3 | Self dimer (kcal mol⁻¹) | −6.76 |
| | deltaG (kcal mol⁻¹) | −144.4 | | |
| | Molecular mass | 49,302 | | |
| OligoTag_4_Series1 | | | | |
| OligoTag_4T_Ser1 | ATC-ATCTGACTTTCGTATCTAGC- | | | |
| (5′ → 3′) | [AGGTTGCTGAGTACGTGAACGGACCACTTGCACT]- | | | |
| SEQ ID NO: 7 | CCATGAGAAGTTCATACACA-TAT | | | |
| OligoTag_4C_Ser1 | ATA-TGTGTATGAACTTCTCATGG- | | | |
| (5′ → 3′) | [AGTGCAAGTGGTCCGTTCACGTACTCAGCAACCT]- | | | |
| SEQ ID NO: 8 | GCTAGATACGAAAGTCAGAT-GAT | | | |
| | Ds oligo: | | Ss oligo: | |
| | Length (bp) | 80 | Hairpin Tm (° C.) | 44.7 |
| | GC (%) | 43.8 | Hairpin deltaG (kcal mol⁻¹) | −5.26 |
| | Tm (° C.) | 88.5 | Self dimer (kcal mol⁻¹) | −7.05 |
| | deltaG (kcal mol⁻¹) | −142.5 | | |
| | Molecular mass | 49,303 | | |
| OligoTag_5_Series1 | | | | |
| OligoTag_5T_Ser1 | ATC-ATCTGACTTTCGTATCTAGC- | | | |
| (5′ → 3′) | [AAGCTCTCGTACAGTGACGCACTACACTCA]- | | | |
| SEQ ID NO: 9 | CCATGAGAAGTTCATACACA-TAT | | | |
| OligoTag_5C_Ser1 | ATA-TGTGTATGAACTTCTCATGG- | | | |
| (5′ → 3′) | [TGAGTGTAGTGCGTCACTGTACGAGAGCTT]- | | | |
| SEQ ID NO: 10 | GCTAGATACGAAAGTCAGAT-GAT | | | |
| | Ds oligo: | | Ss oligo: | |
| | Length (bp) | 76 | Hairpin Tm (° C.) | 44.7 |
| | GC (%) | 41.2 | Hairpin deltaG (kcal mol⁻¹) | −3.29 |
| | Tm (° C.) | 85.5 | Self dimer (kcal mol⁻¹) | −6.34 |
| | deltaG (kcal mol⁻¹) | −130.1 | | |
| | Molecular mass | 46,830 | | |
| OligoTag_6_Series1 | | | | |
| OligoTag_6T_Ser1 | ATC-ATCTGACTTTCGTATCTAGC- | | | |
| (5′ → 3′) | [TCCTAGACGACTGCTACCATCCAAGCGACT]- | | | |
| SEQ ID NO: 11 | CCATGAGAAGTTCATACACA-TAT | | | |
| OligoTag_6C_Ser1 | ATA-TGTGTATGAACTTCTCATGG- | | | |
| (5′ → 3′) | [AGTCGCTTGGATGGTAGCAGTCGTCTAGGA]- | | | |
| SEQ ID NO: 12 | GCTAGATACGAAAGTCAGAT-GAT | | | |
| | Ds oligo: | | Ss oligo: | |
| | Length (bp) | 76 | Hairpin Tm (° C.) | 37.0 |
| | GC (%) | 43.4 | Hairpin deltaG (kcal mol⁻¹) | −3.09 |
| | Tm (° C.) | 86.2 | Self dimer (kcal mol⁻¹) | −6.53 |
| | deltaG (kcal mol⁻¹) | −134.3 | | |
| | Molecular mass | 46,831 | | |
| OligoTag_7_Series1 | | | | |
| OligoTag_7T_Ser1 | ATC-ATCTGACTTTCGTATCTAGC- | | | |
| (5′ → 3′) | [CTTCAGAGAGCACTCTTCATGCAGGTTGCA]- | | | |
| SEQ ID NO: 13 | CCATGAGAAGTTCATACACA-TAT | | | |
| OligoTag_7C_Ser1 | ATA-TGTGTATGAACTTCTCATGG- | | | |
| (5′ → 3′) | [TGCAACCTGCATGAAGAGTGCTCTCTGAAG]- | | | |
| SEQ ID NO: 14 | GCTAGATACGAAAGTCAGAT-GAT | | | |
| | Ds oligo: | | Ss oligo: | |
| | Length (bp) | 76 | Hairpin Tm (° C.) | 45.4 |
| | GC (%) | 42.1 | Hairpin deltaG (kcal mol⁻¹) | −4.73 |
| | Tm (° C.) | 87.2 | Self dimer (kcal mol⁻¹) | −7.05 |
| | deltaG (kcal mol⁻¹) | −134.0 | | |
| | Molecular mass | 46,831 | | |
| OligoTag_8_Series1 | | | | |
| OligoTag_8T_Ser1 | ATC-ATCTGACTTTCGTATCTAGC- | | | |
| (5′ → 3′) | [ATCTCCTGATTGCACTGCTTTGCGTTGCGA]- | | | |
| SEQ ID NO: 15 | CCATGAGAAGTTCATACACA-TAT | | | |
| OligoTag_8C_Ser1 | ATA-TGTGTATGAACTTCTCATGG- | | | |
| (5′ → 3′) | [TCGCAACGCAAAGCAGTGCAATCAGGAGAT]- | | | |
| SEQ ID NO: 16 | GCTAGATACGAAAGTCAGAT-GAT | | | |
| | Ds oligo: | | Ss oligo: | |
| | Length (bp) | 76 | Hairpin Tm (° C.) | 37.8 |
| | GC (%) | 42.1 | Hairpin deltaG (kcal mol⁻¹) | −3.02 |
| | Tm (° C.) | 88.6 | Self dimer (kcal mol⁻¹) | −7.05 |
| | deltaG (kcal mol⁻¹) | −138.3 | | |
| | Molecular mass | 46,830 | | |
| OligoTag_9_Series1 | | | | |
| OligoTag_9T_Ser1 | ATC-ATCTGACTTTCGTATCTAGC- | | | |
| (5′ → 3′) | [TGACCTGAACGTGGACCTCAGACT]-CCATGAGAAGTTCATACACA- | | | |
| SEQ ID NO: 17 | TAT | | | |
| OligoTag_9C_Ser1 | ATA-TGTGTATGAACTTCTCATGG- | | | |
| (5′ → 3′) | [AGTCTGAGGTCCACGTTCAGGTCA]-GCTAGATACGAAAGTCAGAT- | | | |
| SEQ ID NO: 18 | GAT | | | |
| | Ds oligo: | | Ss oligo: | |
| | Length (bp) | 70 | Hairpin Tm (° C.) | 39.3 |
| | GC (%) | 42.9 | Hairpin deltaG (kcal mol⁻¹) | −2.59 |
| | Tm (° C.) | 85.7 | Self dimer (kcal mol⁻¹) | −6.82 |
| | deltaG (kcal mol⁻¹) | −121.1 | | |
| | Molecular mass | 43,124 | | |
| OligoTag_10_Series1 | | | | |
| OligoTag_10T_Ser1 | ATC-ATCTGACTTTCGTATCTAGC- | | | |
| (5′ → 3′) | [ACATCCCTACCAACGCACTACCAG]-CCATGAGAAGTTCATACACA- | | | |
| SEQ ID NO: 19 | TAT | | | |
| OligoTag_10C_Ser1 | ATA-TGTGTATGAACTTCTCATGG- | | | |
| (5′ → 3′) | [CTGGTAGTGCGTTGGTAGGGATGT]-GCTAGATACGAAAGTCAGAT- | | | |
| SEQ ID NO: 20 | GAT | | | |
| | Ds oligo: | | Ss oligo: | |
| | Length (bp) | 70 | Hairpin Tm (° C.) | 32.8 |
| | GC (%) | 42.9 | Hairpin deltaG (kcal mol⁻¹) | −1.29 |
| | Tm (° C.) | 85.8 | Self dimer (kcal mol⁻¹) | −5.38 |
| | deltaG (kcal mol⁻¹) | −124.7 | | |
| | Molecular mass | 43,124 | | |
| OligoTag_11_Series1 | | | | |
| OligoTag_11T_Ser1 | ATC-ATCTGACTTTCGTATCTAGC- | | | |
| (5′ → 3′) | [ACTCGGTTAGCTGCAAGGACCACT]-CCATGAGAAGTTCATACACA- | | | |
| SEQ ID NO: 21 | TAT | | | |
| OligoTag_11C_Ser1 | ATA-TGTGTATGAACTTCTCATGG- | | | |
| (5′ → 3′) | [AGTGGTCCTTGCAGCTAACCGAGT]-GCTAGATACGAAAGTCAGAT- | | | |
| SEQ ID NO: 22 | GAT | | | |
| | Ds oligo: | | Ss oligo: | |
| | Length (bp) | 70 | Hairpin Tm (° C.) | 36.9 |
| | GC (%) | 42.9 | Hairpin deltaG (kcal mol⁻¹) | −2.79 |
| | Tm (° C.) | 85.8 | Self dimer (kcal mol⁻¹) | −7.05 |
| | deltaG (kcal mol⁻¹) | −124.2 | | |
| | Molecular mass | 43,124 | | |
| OligoTag_12_Series1 | | | | |
| OligoTag_12T_Ser1 | ATC-ATCTGACTTTCGTATCTAGC- | | | |
| (5′ → 3′) | [GTTCGTTGCAGGTCTACACGATCA]-CCATGAGAAGTTCATACACA- | | | |
| SEQ ID NO: 23 | TAT | | | |
| OligoTag_12C_Ser1 | ATA-TGTGTATGAACTTCTCATGG- | | | |
| (5′ → 3′) | [TGATCGTGTAGACCTGCAACGAAC]-GCTAGATACGAAAGTCAGAT- | | | |
| SEQ ID NO: 24 | GAT | | | |
| | Ds oligo: | | Ss oligo: | |
| | Length (bp) | 70 | Hairpin Tm (° C.) | 37.7 |
| | GC (%) | 41.4 | Hairpin deltaG (kcal mol⁻¹) | −3.04 |
| | Tm (° C.) | 85.8 | Self dimer (kcal mol⁻¹) | −7.05 |
| | deltaG (kcal mol⁻¹) | −123.1 | | |
| | Molecular mass | 43,123 | | |
| OligoTag_13_Series1 | | | | |
| OligoTag_13T_Ser1 | ATC-ATCTGACTTTCGTATCTAGC-[TTCCTCAGCAGAGTCGGAGT]- | | | |
| (5′ → 3′) | CCATGAGAAGTTCATACACA-TAT | | | |
| SEQ ID NO: 25 | | | | |
| OligoTag_13C_Ser1 | ATA-TGTGTATGAACTTCTCATGG-[ACTCCGACTCTGCTGAGGAA]- | | | |
| (5′ → 3′) | GCTAGATACGAAAGTCAGAT-GAT | | | |
| SEQ ID NO: 26 | | | | |
| | Ds oligo: | | Ss oligo: | |
| | Length (bp) | 66 | Hairpin Tm (° C.) | 44.2 |
| | GC (%) | 42.4 | Hairpin deltaG (kcal mol⁻¹) | −3.22 |
| | Tm (° C.) | 84.4 | Self dimer (kcal mol⁻¹) | −6.34 |
| | deltaG (kcal mol⁻¹) | −114.8 | | |
| | Molecular mass | 40,652 | | |
| OligoTag_14_Series1 | | | | |
| OligoTag_14T_Ser1 | ATC-ATCTGACTTTCGTATCTAGC-[GAGCCACTAGCCTCTGAAC]- | | | |
| (5′ → 3′) | CCATGAGAAGTTCATACACA-TAT | | | |
| SEQ ID NO: 27 | | | | |
| OligoTag_14C_Ser1 | ATA-TGTGTATGAACTTCTCATGG-[GTTCAGAGGCTAGTGGCTCA]- | | | |
| (5′ → 3′) | GCTAGATACGAAAGTCAGAT-GAT | | | |
| SEQ ID NO: 28 | | | | |
| | Ds oligo: | | Ss oligo: | |
| | Length (bp) | 66 | Hairpin Tm (° C.) | 38.4 |
| | GC (%) | 42.4 | Hairpin deltaG (kcal mol⁻¹) | −2.42 |
| | Tm (° C.) | 84.1 | Self dimer (kcal mol⁻¹) | −6.82 |
| | deltaG (kcal mol⁻¹) | −115.6 | | |
| | Molecular mass | 40,652 | | |
| OligoTag_15_Series1 | | | | |
| OligoTag_15T_Ser1 | ATC-ATCTGACTTTCGTATCTAGC-[AGCGTTCACTCGAACCTACA]- | | | |
| (5′ → 3′) | CCATGAGAAGTTCATACACA-TAT | | | |
| SEQ ID NO: 29 | | | | |
| OligoTag_15C_Ser1 | ATA-TGTGTATGAACTTCTCATGG-[TGTAGGTTCGAGTGAACGCT]- | | | |
| (5′ → 3′) | GCTAGATACGAAAGTCAGAT-GAT | | | |
| SEQ ID NO: 30 | | | | |
| | Ds oligo: | | Ss oligo: | |
| | Length (bp) | 66 | Hairpin Tm (° C.) | 40.5 |
| | GC (%) | 40.9 | Hairpin deltaG (kcal mol⁻¹) | −2.85 |
| | Tm (° C.) | 84.1 | Self dimer (kcal mol⁻¹) | −7.13 |
| | deltaG (kcal mol⁻¹) | −114.7 | | |
| | Molecular mass | 40,651 | | |
| OligoTag_16_Series1 | | | | |
| OligoTag_16T_Ser1 | ATC-ATCTGACTTTCGTATCTAGC-[ACCGTGCGAGTGTAGCAAGT]- | | | |
| (5′ → 3′) | CCATGAGAAGTTCATACACA-TAT | | | |
| SEQ ID NO: 31 | | | | |
| OligoTag_16C_Ser1 | ATA-TGTGTATGAACTTCTCATGG-[ACTTGCTACACTCGCACGGT]- | | | |
| (5′ → 3′) | GCTAGATACGAAAGTCAGAT-GAT | | | |
| SEQ ID NO: 32 | | | | |
| | Ds oligo: | | Ss oligo: | |
| | Length (bp) | 66 | Hairpin Tm (° C.) | 37.5 |
| | GC (%) | 42.4 | Hairpin deltaG (kcal moil) | −2.67 |
| | Tm (° C.) | 85.2 | Self dimer (kcal moil) | −6.46 |
| | deltaG (kcal moil) | −116.3 | | |
| | Molecular mass | 40,652 | | |
| OligoTag_17_Series1 | | | | |
| OligoTag_17T_Ser1 | ATC-ATCTGACTTTCGTATCTAGC-[AGGCAGTCGTGCTT]- | | | |
| (5′ → 3′) | CCATGAGAAGTTCATACACA-TAT | | | |
| SEQ ID NO: 33 | | | | |
| OligoTag_17C_Ser1 | ATA-TGTGTATGAACTTCTCATGG-[AAGCACGACTGCCT]- | | | |
| (5′ → 3′) | GCTAGATACGAAAGTCAGAT-GAT | | | |
| SEQ ID NO: 34 | | | | |
| | Ds oligo: | | Ss oligo: | |
| | Length (bp) | 60 | Hairpin Tm (° C.) | 42.7 |
| | GC (%) | 41.7 | Hairpin deltaG (kcal mol⁻¹) | −3.02 |
| | Tm (° C.) | 83.8 | Self dimer (kcal mol⁻¹) | −6.69 |
| | deltaG (kcal mol⁻¹) | −105.7 | | |
| | Molecular mass | 36,945 | | |
| OligoTag_18_Series1 | | | | |
| OligoTag_18T_Ser1 | ATC-ATCTGACTTTCGTATCTAGC-[TGGACCTCGATGCT]- | | | |
| (5′ → 3′) | CCATGAGAAGTTCATACACA-TAT | | | |
| SEQ ID NO: 35 | | | | |
| OligoTag_18C_Ser1 | ATA-TGTGTATGAACTTCTCATGG-[AGCATCGAGGTCCA]- | | | |
| (5′ → 3′) | GCTAGATACGAAAGTCAGAT-GAT | | | |
| SEQ ID NO: 36 | | | | |
| | Ds oligo: | | Ss oligo: | |
| | Length (bp) | 60 | Hairpin Tm (° C.) | 37.8 |
| | GC (%) | 41.7 | Hairpin deltaG (kcal mol⁻¹) | −2.16 |
| | Tm (° C.) | 83.5 | Self dimer (kcal mol⁻¹) | −6.76 |
| | deltaG (kcal mol⁻¹) | −105.1 | | |
| | Molecular mass | 36,945 | | |
| OligoTag_19_Series1 | | | | |
| OligoTag_19T_Ser1 | ATC-ATCTGACTTTCGTATCTAGC-[GAGTAGCACCCTGA]- | | | |
| (5′ → 3′) | CCATGAGAAGTTCATACACA-TAT | | | |
| SEQ ID NO: 37 | | | | |
| OligoTag_19C_Ser1 | ATA-TGTGTATGAACTTCTCATGG-[TCAGGGTGCTACTC]- | | | |
| (5′ → 3′) | GCTAGATACGAAAGTCAGAT-GAT | | | |
| SEQ ID NO: 38 | | | | |
| | Ds oligo: | | Ss oligo: | |
| | Length (bp) | 60 | Hairpin Tm (° C.) | 43.2 |
| | GC (%) | 41.7 | Hairpin deltaG (kcal mol⁻¹) | −2.84 |
| | Tm (° C.) | 82.6 | Self dimer (kcal mol⁻¹) | −5.38 |
| | deltaG (kcal mol⁻¹) | −104.1 | | |
| | Molecular mass | 36,973 | | |
| OligoTag_20_Series1 | | | | |
| OligoTag_20T_Ser1 | ATC-ATCTGACTTTCGTATCTAGC-[CACCTGCTTCCAGA]- | | | |
| (5′ → 3′) | CCATGAGAAGTTCATACACA-TAT | | | |
| SEQ ID NO: 39 | | | | |
| OligoTag_20C_Ser1 | ATA-TGTGTATGAACTTCTCATGG-[TCTGGAAGCAGGTG]- | | | |
| (5′ → 3′) | GCTAGATACGAAAGTCAGAT-GAT | | | |
| SEQ ID NO: 40 | | | | |
| | Ds oligo: | | Ss oligo: | |
| | Length (bp) | 60 | Hairpin Tm (° C.) | 44.9 |
| | GC (%) | 41.7 | Hairpin deltaG (kcal mol⁻¹) | −2.91 |
| | Tm (° C.) | 83.4 | Self dimer (kcal mol⁻¹) | −5.38 |
| | deltaG (kcal mol⁻¹) | −105.1 | | |
| | Molecular mass | 36,945 | | |

Table 2 shows a Gibbs free energy (ΔG) matrix of fragment-fragment interactions between the template (T) and complementary strands (C) of all Series 1 taggants in Table 1 in combination. The Gibbs free energy of a reaction is the change in enthalpy minus the product of the temperature and the change in entropy. The more negative that ΔG is, the greater the tendency towards cross-fragment hybridisation and the higher the annealing temperature. The ΔG of the dsDNA taggant duplexes of length 60-80 bp with perfect complementarity ranges between −104.1 and −144.4 kcal mol⁻¹ (shown in bold in Table 2). Although binding between the template and complementary strands of the same taggants reduces PCR efficiency, this is not a problem since complete complementarity does not result in strand elongation.

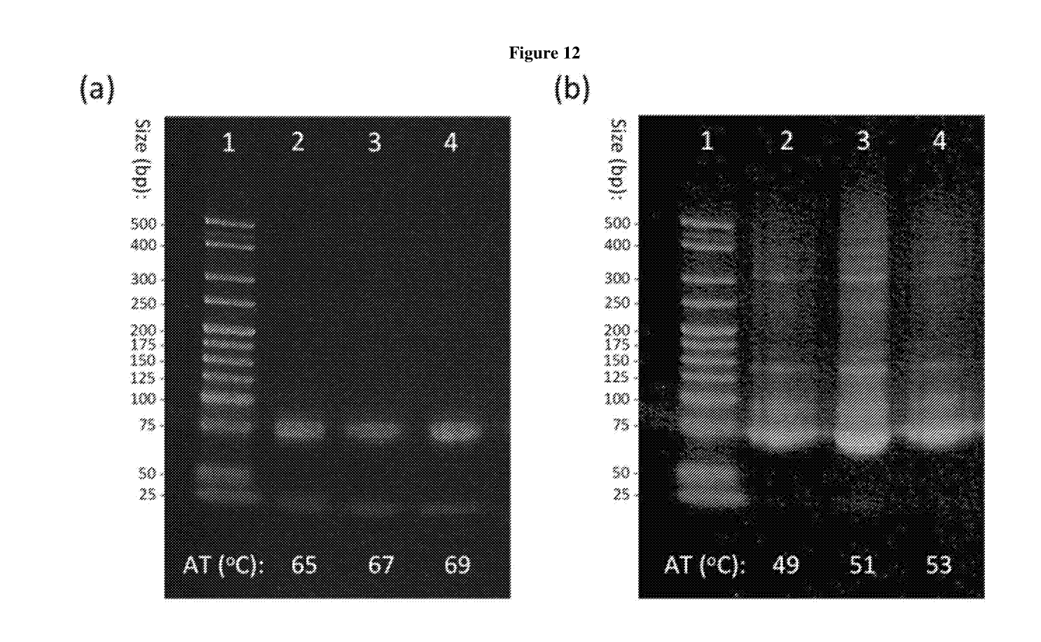

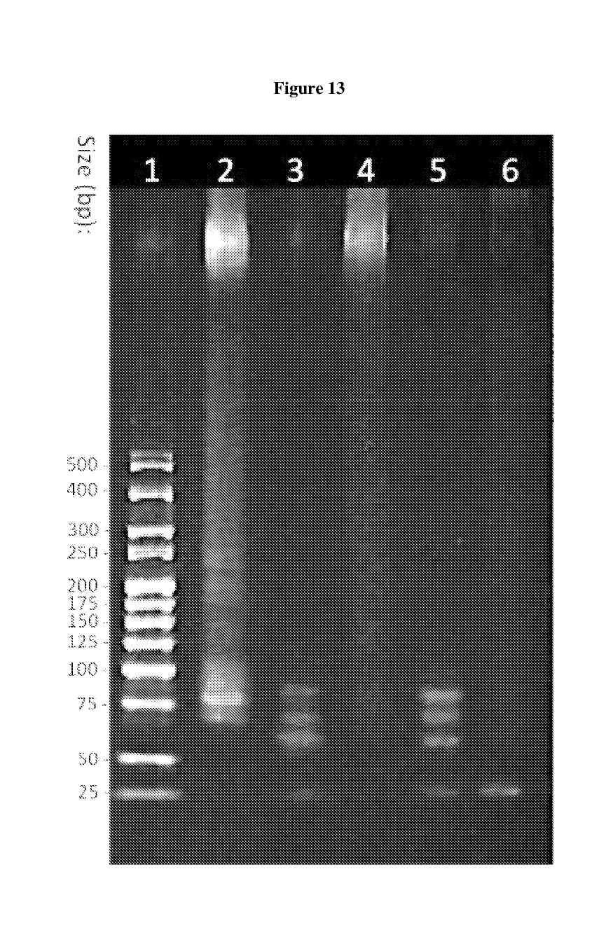

Table 2 also shows that the ΔG of cross-fragment interactions of the 20 taggants range between −44.5 and −37.9 kcal mol⁻¹. This range is typical for 60-80 bp oligonucleotide fragments that share common forward and reverse primer sites, but is also problematic since conventional primer-taggant binding occurs at a less negative ΔG of −32.7 kcal mol⁻¹. Design specifications commonly recommend that self-dimers, hairpins, and heterodimer formation should be weaker (ie. less negative) than −9 kcal mol⁻¹. The diffusivity of these short 60-80 bp fragments, due to Brownian motion, is also similar to that of the primers, which increases the probability of cross-fragment priming and hybridization in solution. Extensive cross-fragment hybridization is predicted, and observed, during conventional PCR amplification of the mixed population taggants in Table 1 based on the ΔG values given in Table 2. This is shown in FIG. 12 and FIG. 13. As it is not possible to design 60-100 bp fragments with common primer sequences and ΔG≥−9 kcal mol⁻¹ (i.e., less negative), the inventor used LNA primers and ATD PCR to reduce (i.e., more negative) the ΔG of primer-fragment interactions relative to fragment-fragment interactions.

TABLE 2
Gibbs free energy of reactions (ΔG, kcal mol⁻¹) between a mixed population of
single stranded fragments with common forward and reverse primer sequences.

| | 1 C | 2 C | 3 C | 4 C | 5 C | 6 C | 7 C | 8 C | 9 C | 10 C | 11 C |
|---|---|---|---|---|---|---|---|---|---|---|---|
| 1 T | −142.8 | −39.8 | −39.8 | −44.5 | −41.4 | −37.9 | −39.8 | −39.8 | −37.9 | −39.8 | −39.8 |
| 2 T | −39.8 | −138.8 | −41.1 | −39.8 | −39.8 | −37.9 | −39.8 | −39.8 | −37.9 | −41.1 | −41.1 |
| 3 T | −39.8 | −41.1 | −144.4 | −39.8 | −39.8 | −38.1 | −37.9 | −39.8 | −38.1 | −43.1 | −41.1 |
| 4 T | −44.5 | −39.8 | −39.8 | −142.6 | −39.8 | −41.0 | −37.9 | −39.8 | −41.0 | −39.8 | −43.0 |
| 5 T | −41.4 | −39.8 | −39.8 | −39.8 | −130.1 | −37.9 | −39.8 | −39.8 | −37.9 | −39.8 | −39.8 |
| 6 T | −37.9 | −37.9 | −38.1 | −41.0 | −37.9 | −134.3 | −37.9 | −37.9 | −42.6 | −37.9 | −41.0 |
| 7 T | −39.8 | −39.8 | −37.9 | −37.9 | −39.8 | −37.9 | −134.0 | −37.9 | −37.9 | −37.9 | −37.9 |
| 8 T | −39.8 | −39.8 | −39.8 | −39.8 | −39.8 | −37.9 | −37.9 | −138.3 | −37.9 | −39.8 | −39.8 |
| 9 T | −37.9 | −37.9 | −38.1 | −41.0 | −37.9 | −42.6 | −37.9 | −37.9 | −121.1 | −37.9 | −41.0 |
| 10 T | −39.8 | −41.1 | −43.1 | −39.8 | −39.8 | −37.9 | −37.9 | −39.8 | −37.9 | −124.7 | −41.1 |
| 11 T | −39.8 | −41.1 | −41.1 | −43.0 | −39.8 | −41.0 | −37.9 | −39.8 | −41.0 | −41.1 | −124.2 |
| 12 T | −41.4 | −39.8 | −37.9 | −37.9 | −41.4 | −37.9 | −39.8 | −37.9 | −37.9 | −37.9 | −37.9 |
| 13 T | −37.9 | −37.9 | −38.1 | −38.1 | −37.9 | −39.4 | −37.9 | −37.9 | −39.4 | −37.9 | −38.1 |
| 14 T | −37.9 | −37.9 | −37.9 | −37.9 | −37.9 | −39.4 | −37.9 | −37.9 | −43.0 | −37.9 | −37.9 |
| 15 T | −41.4 | −43.7 | −39.8 | −41.4 | −39.8 | −37.9 | −39.8 | −39.8 | −37.9 | −39.8 | −39.8 |
| 16 T | −39.8 | −44.2 | −41.1 | −39.8 | −39.8 | −38.1 | −37.9 | −39.8 | −38.1 | −41.1 | −41.1 |
| 17 T | −44.5 | −39.8 | −39.8 | −44.5 | −39.8 | −38.1 | −37.9 | −39.8 | −38.1 | −39.8 | −39.8 |
| 18 T | −37.9 | −37.9 | −38.1 | −39.7 | −37.9 | −39.7 | −37.9 | −37.9 | −41.4 | −37.9 | −39.7 |
| 19 T | −37.9 | −37.9 | −37.9 | −37.9 | −37.9 | −37.9 | −37.9 | −39.4 | −37.9 | −37.9 | −37.9 |
| 20 T | −37.9 | −37.9 | −37.9 | −37.9 | −37.9 | −37.9 | −40.9 | −39.4 | −37.9 | −37.9 | −37.9 |
| | | | 12 C | 13 C | 14 C | 15 C | 16 C | 17 C | 18 C | 19 C | 20 C |
| | | 1 T | −41.4 | −37.9 | −37.9 | −41.4 | −39.8 | −44.5 | −37.9 | −37.9 | −37.9 |
| | | 2 T | −39.8 | −37.9 | −37.9 | −43.7 | −44.2 | −39.8 | −37.9 | −37.9 | −37.9 |
| | | 3 T | −37.9 | −38.1 | −37.9 | −39.8 | −41.1 | −39.8 | −38.1 | −37.9 | −37.9 |
| | | 4 T | −37.9 | −38.1 | −37.9 | −41.4 | −39.8 | −44.5 | −39.7 | −37.9 | −37.9 |
| | | 5 T | −41.4 | −37.9 | −37.9 | −39.8 | −39.8 | −39.8 | −37.9 | −37.9 | −37.9 |
| | | 6 T | −37.9 | −39.4 | −39.4 | −37.9 | −38.1 | −38.1 | −39.7 | −37.9 | −37.9 |
| | | 7 T | −39.8 | −37.9 | −37.9 | −39.8 | −37.9 | −37.9 | −37.9 | −37.9 | −40.9 |
| | | 8 T | −37.9 | −37.9 | −37.9 | −39.8 | −39.8 | −39.8 | −37.9 | −39.4 | −39.4 |
| | | 9 T | −37.9 | −39.4 | −43.0 | −37.9 | −38.1 | −38.1 | −41.4 | −37.9 | −37.9 |
| | | 10 T | −37.9 | −37.9 | −37.9 | −39.8 | −41.1 | −39.8 | −37.9 | −37.9 | −37.9 |
| | | 11 T | −37.9 | −38.1 | −37.9 | −39.8 | −41.1 | −39.8 | −39.7 | −37.9 | −37.9 |
| | | 12 T | −123.1 | −37.9 | −37.9 | −39.8 | −37.9 | −37.9 | −37.9 | −41.5 | −37.9 |
| | | 13 T | −37.9 | −114.8 | −39.4 | −37.9 | −41.0 | −38.1 | −39.4 | −37.9 | −37.9 |
| | | 14 T | −37.9 | −39.4 | −115.6 | −37.9 | −37.9 | −37.9 | −41.4 | −37.9 | −37.9 |
| | | 15 T | −39.8 | −37.9 | −37.9 | −114.7 | −39.8 | −41.4 | −37.9 | −37.9 | −37.9 |
| | | 16 T | −37.9 | −41.0 | −37.9 | −39.8 | −116.3 | −39.8 | −38.1 | −37.9 | −37.9 |
| | | 17 T | −37.9 | −38.1 | −37.9 | −41.4 | −39.8 | −105.7 | −38.1 | −37.9 | −37.9 |
| | | 18 T | −37.9 | −39.4 | −41.4 | −37.9 | −38.1 | −38.1 | −105.1 | −37.9 | −37.9 |
| | | 19 T | −41.5 | −37.9 | −37.9 | −37.9 | −37.9 | −37.9 | −37.9 | −104.1 | −39.4 |
| | | 20 T | −37.9 | −37.9 | −37.9 | −37.9 | −37.9 | −37.9 | −37.9 | −39.4 | −105.1 |

The data show the ΔG of fragment-fragment interactions between single stranded OligoTags_1-20_Ser1.
Abbreviations include: template strand (T), complementary strand (C).

### Example 2—Molecular Taggant Design and Preparation for Series 2 Experiments (.22 Calibre Rifle, .207 Calibre Rifle, and Pharmaceuticals Labelling)

In Series 2 experiments (.22 and .207 caliber firearm ammunition tracing and pharmaceuticals labelling) the taggants were similarly designed in accordance with UniKey-Tag embodiment 1. Specifically, the taggants were designed with no (ie. 0-3 bp) end capping regions, and universal forward and reverse primer sites (22 bp) that flank a variable encoding region of length 46 bp. One difference compared to Series 1 experiments, but still consistent with UniKey-Tag embodiment 1, is that the variable region was assembled from six Hamming (1, d, p) encoded blocks (abbreviated: Ham(l, d, p)).

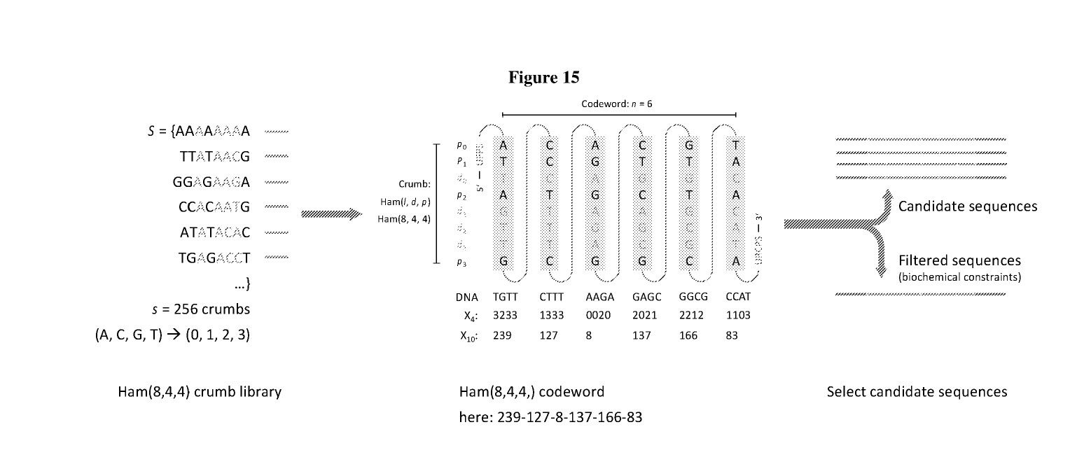

Specifically, the variable regions of taggants in Series 2 experiments were constructed from six Hamming (l, d, p) encoded crumbs (equivalent to binary bytes) of symbol length l=8, including d=4 data and p=4 parity nucleotides, ie. Ham(8,4,4) code. The library of Ham(8,4,4) crumbs used to construct Series 2 codewords is given in Table 3, and the process of codeword assembly is illustrated in FIG. 15 where the vertical blocks show the position of data and parity nucleotides in each l-length quaternary crumb in a string of crumbs that comprise a n-length code word. The nucleotide set Qₙ={A, C, G, T} was mapped to the numeral set Q_{d}={0, 1, 2, 3} so that the DNA data quadruplet TGTT, for example, encodes the quaternary number X₄=3233 which is equivalent to decimal number X₁₀=239. The Ham(8,4,4) crumbs were selected from a library of 256 crumbs that encoded the decimal number set 0 to 255, ie. {0, 1, 2, . . . , 255} given in Table 3. Each crumb in Table 3 is separated by a mutual distance of 4 nucleotides.

In this design, the variable region was encoded with a string of six symbols that was used to lookup information associated with the codeword on a separate database. In a real-life context, this information may include personal identification information such as the license number, permit number, or place of purchase information (ie. for ammunition fingerprinting) or batch number, barcode number, manufacturing date, expiry date, manufacturing facility, manufacturer, product type, etc. (for product tracing, ie pharmaceuticals). This n6-Ham(8,4,4) encoding design permits 2.81×10¹⁴ unique taggant sequences (sⁿ=256⁶) for each universal primer site encoded library, which is essentially unlimited for most practical applications.

In the Series 2 experiments, 10 dsDNA taggants were constructed from six Ham(8,4,4) crumbs that were randomly selected from the 256 crumb library. These blocks were assembled into a codewords of length n=6 that are given in Table 4. This meant that the encoding region for each taggant was 48 bp long (ie. 6×8=48 bp).

The 10 sets of complementary ssDNA oligonucleotides (ie. 20 in total) were annealed to form 10 dsDNA taggant duplexes (OligoTag_1_Ser2 to OligoTag_10_Ser2). The ssDNA oligonucleotides were synthesised by Sigma-Aldrich and purified using high-performance liquid chromatography (HPLC). Production was performed at several different locations and distributed over several weeks to ensure against cross contamination at the manufacturing facility.

The Ham(8,4,4) crumb library is given in Table 3. The sequences and specifications of the Ham(8,4,4) encoded taggants used in Series 2 experiment are given in Table 4. Series 2 experiments include ammunition tracing (for .22 and .207 calibre firearms), and pharmaceuticals labelling. The universal primer sequences used in Series 2 experiments are given in Table 6.

TABLE 3
Hamming(8,4,4) crumb library used to encode
taggants used in Series 2 experiments

| Dec | Ham(8,4,4) |
|---|---|
| 0 | AAAAAAAA |
| 1 | TTATAACG |
| 2 | GGAGAAGA |
| 3 | CCACAATG |
| 4 | ATATACAC |
| 5 | TGAGACCT |
| 6 | GCACACGC |
| 7 | CAAAACTT |
| 8 | AGAGAGAG |
| 9 | TCACAGCA |
| 10 | GAAAAGGG |
| 11 | CTATAGTA |
| 12 | ACACATAT |
| 13 | TAAAATCC |
| 14 | GTATATGT |
| 15 | CGAGATTC |
| 16 | TAATCAAC |
| 17 | GTAGCACT |
| 18 | CGACCAGC |
| 19 | ACAACATT |
| 20 | TTAGCCAG |
| 21 | GGACCCCA |
| 22 | CCAACCGG |
| 23 | AAATCCTA |
| 24 | TGACCGAT |
| 25 | GCAACGCC |
| 26 | CAATCGGT |
| 27 | ATAGCGTC |
| 28 | TCAACTAA |
| 29 | GAATCTCG |
| 30 | CTAGCTGA |
| 31 | AGACCTTG |
| 32 | GAAGGAAG |
| 33 | CTACGACA |
| 34 | AGAAGAGG |
| 35 | TCATGATA |
| 36 | GTACGCAT |
| 37 | CGAAGCCC |
| 38 | ACATGCGT |
| 39 | TAAGGCTC |
| 40 | GGAAGGAA |
| 41 | CCATGGCG |
| 42 | AAAGGGGA |
| 43 | TTACGGTG |
| 44 | GCATGTAC |
| 45 | CAAGGTCT |
| 46 | ATACGTGC |
| 47 | TGAAGTTT |
| 48 | CAACTAAT |
| 49 | ATAATACC |
| 50 | TGATTAGT |
| 51 | GCAGTATC |
| 52 | CTAATCAA |
| 53 | AGATTCCG |
| 54 | TCAGTCGA |
| 55 | GAACTCTG |
| 56 | CGATTGAC |
| 57 | ACAGTGCT |
| 58 | TAACTGGC |
| 59 | GTAATGTT |
| 60 | CCAGTTAG |
| 61 | AAACTTCA |
| 62 | TTAATTGG |
| 63 | GGATTTTA |
| 64 | TTCAAAAC |
| 65 | GGCTAACT |
| 66 | CCCGAAGC |
| 67 | AACCAATT |
| 68 | TGCTACAG |
| 69 | GCCGACCA |
| 70 | CACCACGG |
| 71 | ATCAACTA |
| 72 | TCCGAGAT |
| 73 | GACCAGCC |
| 74 | CTCAAGGT |
| 75 | AGCTAGTC |
| 76 | TACCATAA |
| 77 | GTCAATCG |
| 78 | CGCTATGA |
| 79 | ACCGATTG |
| 80 | GTCTCAAG |
| 81 | CGCGCACA |
| 82 | ACCCCAGG |
| 83 | TACACATA |
| 84 | GGCGCCAT |
| 85 | CCCCCCCC |
| 86 | AACACCGT |
| 87 | TTCTCCTC |
| 88 | GCCCCGAA |
| 89 | CACACGCG |
| 90 | ATCTCGGA |
| 91 | TGCGCGTG |
| 92 | GACACTAC |
| 93 | CTCTCTCT |
| 94 | AGCGCTGC |
| 95 | TCCCCTTT |
| 96 | CTCGGAAT |
| 97 | AGCCGACC |
| 98 | TCCAGAGT |
| 99 | GACTGATC |
| 100 | CGCCGCAA |
| 101 | ACCAGCCG |
| 102 | TACTGCGA |
| 103 | GTCGGCTG |
| 104 | CCCAGGAC |
| 105 | AACTGGCT |
| 106 | TTCGGGGC |
| 107 | GGCCGGTT |
| 108 | CACTGTAG |
| 109 | ATCGGTCA |
| 110 | TGCCGTGG |
| 111 | GCCAGTTA |
| 112 | ATCCTAAA |
| 113 | TGCATACG |
| 114 | GCCTTAGA |
| 115 | CACGTATG |
| 116 | AGCATCAC |
| 117 | TCCTTCCT |
| 118 | GACGTCGC |
| 119 | CTCCTCTT |
| 120 | ACCTTGAG |
| 121 | TACGTGCA |
| 122 | GTCCTGGG |
| 123 | CGCATGTA |
| 124 | AACGTTAT |
| 125 | TTCCTTCC |
| 126 | GGCATTGT |
| 127 | CCCTTTTC |
| 128 | GGGAAAAG |
| 129 | CCGTAACA |
| 130 | AAGGAAGG |
| 131 | TTGCAATA |
| 132 | GCGTACAT |
| 133 | CAGGACCC |
| 134 | ATGCACGT |
| 135 | TGGAACTC |
| 136 | GAGGAGAA |
| 137 | CTGCAGCG |
| 138 | AGGAAGGA |
| 139 | TCGTAGTG |
| 140 | GTGCATAC |
| 141 | CGGAATCT |
| 142 | ACGTATGC |
| 143 | TAGGATTT |
| 144 | CGGTCAAT |
| 145 | ACGGCACC |
| 146 | TAGCCAGT |
| 147 | GTGACATC |
| 148 | CCGGCCAA |
| 149 | AAGCCCCG |
| 150 | TTGACCGA |
| 151 | GGGTCCTG |
| 152 | CAGCCGAC |
| 153 | ATGACGCT |
| 154 | TGGTCGGC |
| 155 | GCGGCGTT |
| 156 | CTGACTAG |
| 157 | AGGTCTCA |
| 158 | TCGGCTGG |
| 159 | GAGCCTTA |
| 160 | AGGGGAAA |
| 161 | TCGCGACG |
| 162 | GAGAGAGA |
| 163 | CTGTGATG |
| 164 | ACGCGCAC |
| 165 | TAGAGCCT |
| 166 | GTGTGCGC |
| 167 | CGGGGCTT |
| 168 | AAGAGGAG |
| 169 | TTGTGGCA |
| 170 | GGGGGGGG |
| 171 | CCGCGGTA |
| 172 | ATGTGTAT |
| 173 | TGGGGTCC |
| 174 | GCGCGTGT |
| 175 | CAGAGTTC |
| 176 | TGGCTAAC |
| 177 | GCGATACT |
| 178 | CAGTTAGC |
| 179 | ATGGTATT |
| 180 | TCGATCAG |
| 181 | GAGTTCCA |
| 182 | CTGGTCGG |
| 183 | AGGCTCTA |
| 184 | TAGTTGAT |
| 185 | GTGGTGCC |
| 186 | CGGCTGGT |
| 187 | ACGATGTC |
| 188 | TTGGTTAA |
| 189 | GGGCTTCG |
| 190 | CCGATTGA |
| 191 | AAGTTTTG |
| 192 | CCTAAAAT |
| 193 | AATTAACC |
| 194 | TTTGAAGT |
| 195 | GGTCAATC |
| 196 | CATTACAA |
| 197 | ATTGACCG |
| 198 | TGTCACGA |
| 199 | GCTAACTG |
| 200 | CTTGAGAC |
| 201 | AGTCAGCT |
| 202 | TCTAAGGC |
| 203 | GATTAGTT |
| 204 | CGTCATAG |
| 205 | ACTAATCA |
| 206 | TATTATGG |
| 207 | GTTGATTA |
| 208 | ACTTCAAA |
| 209 | TATGCACG |
| 210 | GTTCCAGA |
| 211 | CGTACATG |
| 212 | AATGCCAC |
| 213 | TTTCCCCT |
| 214 | GGTACCGC |
| 215 | CCTTCCTT |
| 216 | ATTCCGAG |
| 217 | TGTACGCA |
| 218 | GCTTCGGG |
| 219 | CATGCGTA |
| 220 | AGTACTAT |
| 221 | TCTTCTCC |
| 222 | GATGCTGT |
| 223 | CTTCCTTC |
| 224 | TCTGGAAC |
| 225 | GATCGACT |
| 226 | CTTAGAGC |
| 227 | AGTTGATT |
| 228 | TATCGCAG |
| 229 | GTTAGCCA |
| 230 | CGTTGCGG |
| 231 | ACTGGCTA |
| 232 | TTTAGGAT |
| 233 | GGTTGGCC |
| 234 | CCTGGGGT |
| 235 | AATCGGTC |
| 236 | TGTTGTAA |
| 237 | GCTGGTCG |
| 238 | CATCGTGA |
| 239 | ATTAGTTG |
| 240 | GCTCTAAG |
| 241 | CATATACA |
| 242 | ATTTTAGG |
| 243 | TGTGTATA |
| 244 | GATATCAT |
| 245 | CTTTTCCC |
| 246 | AGTGTCGT |
| 247 | TCTCTCTC |
| 248 | GTTTTGAA |
| 249 | CGTGTGCG |
| 250 | ACTCTGGA |
| 251 | TATATGTG |
| 252 | GGTGTTAC |
| 253 | CCTCTTCT |
| 245 | AATATTGC |
| 255 | TTTTTTTT |

TABLE 4
Fragment sequences and specifications used in Series 2 experiments.

| OligoTag_1_Series2 | | | | |
|---|---|---|---|---|
| Codeword: | | | | |
| 52-45-117-193-159-125 | | | | |
| OligoTag_1T_Ser2 | TTTCTGTTGGTGCTGATATTGC-[CTAATCAA-CAAGGTCT- | | | |
| (5′ → 3′) | TCCTTCCT-AATTAACC-GAGCCTTA-TTCCTTCC]- | | | |
| SEQ ID NO: 41 | GAAGATAGAGCGACAGGCAAGT | | | |
| OligoTag_1C_Ser2 | ACTTGCCTGTCGCTCTATCTTC-[GGAAGGAA-TAAGGCTC- | | | |
| (5′ → 3′) | GGTTAATT-AGGAAGGA-AGACCTTG-TTGATTAG]- | | | |
| SEQ ID NO: 42 | GCAATATCAGCACCAACAGAAA | | | |
| | Ds oligo: | | Ss oligo: | |
| | Length (bp) | 92 | Hairpin Tm (° C.) | 35.9 |
| | GC (%) | 43.5 | Hairpin deltaG (kcal mol⁻¹) | −3.46 |
| | Tm (° C.) | 70.9 | Self dimer (kcal mol⁻¹) | −11.71 |
| | deltaG (kcal mol⁻¹) | −172.32 | | |
| | Molecular mass | 56,502.2 | | |
| OligoTag_2_Series2 | | | | |
| Codeword: | | | | |
| 11-187-105-134-210-56 | | | | |
| OligoTag_2T_Ser2 | TTTCTGTTGGTGCTGATATTGC-[CTATAGTA-ACGATGTC- | | | |
| (5′ → 3′) | AACTGGCT-ATGCACGT-GTTCCAGA-CGATTGAC]- | | | |
| SEQ ID NO: 43 | GAAGATAGAGCGACAGGCAAGT | | | |
| OligoTag_2C_Ser2 | ACTTGCCTGTCGCTCTATCTTC-[GTCAATCG-TCTGGAAC- | | | |
| (5′ → 3′) | ACGTGCAT-AGCCAGTT-GACATCGT-TACTATAG]- | | | |
| SEQ ID NO: 44 | GCAATATCAGCACCAACAGAAA | | | |
| | Ds oligo: | | Ss oligo: | |
| | Length (bp) | 92 | Hairpin Tm (° C.) | 45.1 |
| | GC (%) | 45.7 | Hairpin deltaG (kcal mol⁻¹) | −5.12 |
| | Tm (° C.) | 71.3 | Self dimer (kcal mol⁻¹) | −11.71 |
| | deltaG (kcal mol⁻¹) | −172.61 | | |
| | Molecular mass | 57,021 | | |
| OligoTag_3_Series2 | | | | |
| Codeword: | | | | |
| 4-105-238-120-5-132 | | | | |
| OligoTag_3T_Ser2 | TTTCTGTTGGTGCTGATATTGC-[ATATACAC-AACTGGCT- | | | |
| (5′ → 3′) | CATCGTGA-ACCTTGAG-TGAGACCT-GCGTACAT]- | | | |
| SEQ ID NO: 45 | GAAGATAGAGCGACAGGCAAGT | | | |
| OligoTag_3C_Ser2 | ACTTGCCTGTCGCTCTATCTTC-[ATGTACGC-AGGTCTCA- | | | |
| (5′ → 3′) | CTCAAGGT-TCACGATG-AGCCAGTT-GTGTATAT]- | | | |
| SEQ ID NO: 46 | GCAATATCAGCACCAACAGAAA | | | |
| | Ds oligo: | | Ss oligo: | |
| | Length (bp) | 92 | Hairpin Tm (° C.) | 35.5 |
| | GC (%) | 45.7 | Hairpin deltaG (kcal mol⁻) | −4.46 |
| | Tm (° C.) | 71.4 | Self dimer (kcal mol⁻¹) | −7.05 |
| | deltaG (kcal mol⁻¹) | −169.94 | | |
| | Molecular mass | 56,959 | | |
| OligoTag_4_Series2 | | | | |
| Codeword: | | | | |
| 90-14-239-168-206-163 | | | | |
| OligoTag_4T_5er2 | TTTCTGTTGGTGCTGATATTGC-[ATCTCGGA-GTATATGT- | | | |
| (5′ → 3′) | ATTAGTTG-AAGAGGAG-TATTATGG-CTGTGATG]- | | | |
| SEQ ID NO: 47 | GAAGATAGAGCGACAGGCAAGT | | | |
| OligoTag_4C_Ser2 | ACTTGCCTGTCGCTCTATCTTC-[CATCACAG-CCATAATA- | | | |
| (5′ → 3′) | CTCCTCTTC-AACTAATA-CATATACT-CCGAGAT]- | | | |
| SEQ ID NO: 48 | GCAATATCAGCACCAACAGAAA | | | |
| | Ds oligo: | | Ss oligo: | |
| | Length (bp) | 92 | Hairpin Tm (° C.) | 41.8 |
| | GC (%) | 41.3 | Hairpin deltaG (kcal mol⁻¹) | −4.32 |
| | Tm (° C.) | 70 | Self dimer (kcal mol⁻¹) | −7.05 |
| | deltaG (kcal mol⁻¹) | −165.5 | | |
| | Molecular mass | 57,461.2 | | |
| OligoTag_5_Series2 | | | | |
| Codeword: | | | | |
| 253-109-80-45-34-78 | | | | |
| OligoTag_5T_Ser2 | TTTCTGTTGGTGCTGATATTGC- | | | |
| (5′ → 3′) | [CCTCTTCTATCGGTCAGTCTCAAGCAAGGTCTAGAAGAGGCGCTA | | | |
| SEQ ID NO: 49 | TGA]-GAAGATAGAGCGACAGGCAAGT | | | |
| OligoTag_5C_Ser2 | ACTTGCCTGTCGCTCTATCTTC-[TCATAGCG-CCTCTTCT- | | | |
| (5′ → 3′) | AGACCTTG-CTTGAGAC-TGACCGAT-AGAAGAGG]- | | | |
| SEQ ID NO: 50 | GCAATATCAGCACCAACAGAAA | | | |
| | Ds oligo: | | Ss oligo: | |
| | Length (bp) | 92 | Hairpin Tm (° C.) | 49.2 |
| | GC (%) | 47.8 | Hairpin deltaG (kcal mol⁻¹) | −7.72 |
| | Tm (° C.) | 71.8 | Self dimer (kcal mol⁻¹) | −13.92 |
| | deltaG (kcal mol⁻¹) | −173.97 | | |
| | Molecular mass | 57,023 | | |
| OligoTag_6_Series2 | | | | |
| Codeword: | | | | |
| 62-3-211-253-221-227 | | | | |
| OligoTag_6T_Ser2 | TTTCTGTTGGTGCTGATATTGC-[TTAATTGG-CCACAATG- | | | |
| (5′ → 3′) | CGTACATG-CCTCTTCT-TCTTCTCC-AGTTGATT]- | | | |
| SEQ ID NO: 51 | GAAGATAGAGCGACAGGCAAGT | | | |
| OligoTag_6C_Ser2 | ACTTGCCTGTCGCTCTATCTTC-[AATCAACT-GGAGAAGA- | | | |
| (5′ → 3′) | AGAAGAGG-CATGTACG-CATTGTGG-CCAATTAA]- | | | |
| SEQ ID NO: 52 | GCAATATCAGCACCAACAGAAA | | | |
| | Ds oligo: | | Ss oligo: | |
| | Length (bp) | 92 | Hairpin Tm (° C.) | 37.9 |
| | GC (%) | 43.5 | Hairpin deltaG (kcal mol⁻¹) | −4.71 |
| | Tm (° C.) | 71.3 | Self dimer (kcal mol⁻¹) | −13.19 |
| | deltaG (kcal mol⁻¹) | −174.22 | | |
| | Molecular mass | 56,688.8 | | |
| OligoTag_7_Series2 | | | | |
| Codeword: | | | | |
| 226-49-33-205-59-176 | | | | |
| OligoTag_7T_Ser2 | TTTCTGTTGGTGCTGATATTGC-[CTTAGAGC-ATAATACC- | | | |
| (5′ → 3′) | CTACGACA-ACTAATCA-GTAATGTT-TGGCTAAC]- | | | |
| SEQ ID NO: 53 | GAAGATAGAGCGACAGGCAAGT | | | |
| OligoTag_7C_Ser2 | ACTTGCCTGTCGCTCTATCTTC-[GTTAGCCA-AACATTAC- | | | |
| (5′ → 3′) | TGATTAGT-TGTCGTAG-GGTATTAT-GCTCTAAG]- | | | |
| SEQ ID NO: 54 | GCAATATCAGCACCAACAGAAA | | | |
| | Ds oligo: | | Ss oligo: | |
| | Length (bp) | 92 | Hairpin Tm (° C.) | 36.9 |
| | GC (%) | 41.3 | Hairpin deltaG (kcal mol⁻¹) | −4.21 |
| | Tm (° C.) | 70.2 | Self dimer (kcal mol⁻¹) | −11.71 |
| | deltaG (kcal mol⁻¹) | −169.04 | | |
| | Molecular mass | 56,893 | | |
| OligoTag_8_Series2 | | | | |
| Codeword: | | | | |
| 251-83-203-36-86-129 | | | | |
| OligoTag_8T_Ser2 | TTTCTGTTGGTGCTGATATTGC- | | | |
| (5′ → 3′) | [TATATGTGTACACATAGATTAGTTGTACGCATAACACCGTCCGTA | | | |
| SEQ ID NO: 55 | ACA]-GAAGATAGAGCGACAGGCAAGT | | | |
| OligoTag_8C_Ser2 | ACTTGCCTGTCGCTCTATCTTC-[TGTTACGG-ACGGTGTT- | | | |
| (5′ → 3′) | ATGCGTAC-AACTAATC-TATGTGTA-CACATATA]- | | | |
| SEQ ID NO: 56 | GCAATATCAGCACCAACAGAAA | | | |
| | Ds oligo: | | Ss oligo: | |
| | Length (bp) | 92 | Hairpin Tm (° C.) | 43 |
| | GC (%) | 41.3 | Hairpin deltaG (kcal mol⁻¹) | −6.32 |
| | Tm (° C.) | 70 | Self dimer (kcal mol⁻¹) | −19.02 |
| | deltaG (kcal mol⁻¹) | −164.23 | | |
| | Molecular mass | 56,955 | | |
| OligoTag_9_Series2 | | | | |
| Codeword: | | | | |
| 75-4-236-3-16-153 | | | | |
| OligoTag_9T_Ser2 | TTTCTGTTGGTGCTGATATTGC-[AGCTAGTC-ATATACAC- | | | |
| (5′ → 3′) | TGTTGTAA-CCACAATG-TAATCAAC-ATGACGCT]- | | | |
| SEQ ID NO: 57 | GAAGATAGAGCGACAGGCAAGT | | | |
| OligoTag_9C_Ser2 | ACTTGCCTGTCGCTCTATCTTC-[AGCGTCAT-GTTGATTA- | | | |
| (5′ → 3′) | CATTGTGG-TTACAACA-GTGTATAT-GACTAGCT]- | | | |
| SEQ ID NO: 58 | GCAATATCAGCACCAACAGAAA | | | |
| | Ds oligo: | | Ss oligo: | |
| | Length (bp) | 92 | Hairpin Tm (° C.) | 47.9 |
| | GC (%) | 41.3 | Hairpin deltaG (kcal mol⁻¹) | −9.06 |
| | Tm (° C.) | 70.5 | Self dimer (kcal mol⁻¹) | −8.35 |
| | deltaG (kcal mol⁻¹) | −164.45 | | |
| | Molecular mass | 56,893 | | |
| OligoTag_10_Series2 | | | | |
| Codeword: | | | | |
| 117-2-240-199-157-252 | | | | |
| OligoTag_10T_Ser2 | TTTCTGTTGGTGCTGATATTGC-[TCCTTCCT-GGAGAAGA- | | | |
| (5′ → 3′) | GCTCTAAG-GCTAACTG-AGGTCTCA-GGTGTTAC]- | | | |
| SEQ ID NO: 59 | GAAGATAGAGCGACAGGCAAGT | | | |
| OligoTag_10C_Ser2 | ACTTGCCTGTCGCTCTATCTTC-[GTAACACC-TGAGACCT- | | | |
| (5′ → 3′) | CAGTTAGC-CTTAGAGC-TCTTCTCC-AGGAAGGA]- | | | |
| SEQ ID NO: 60 | GCAATATCAGCACCAACAGAAA | | | |
| | Ds oligo: | | Ss oligo: | |
| | Length (bp) | 92 | Hairpin Tm (° C.) | 49.8 |
| | GC (%) | 47.8 | Hairpin deltaG (kcal mol⁻¹) | −9.42 |
| | Tm (° C.) | 71.5 | Self dimer (kcal mol⁻¹) | −12.68 |
| | deltaG (kcal mol⁻¹) | −171.99 | | |
| | Molecular mass | 57,085 | | |

### Example 3—Design of Base Pair-Encoded Taggants (UniKey-Tag 1)

For the UniKey-Tag 1 system, each symbol (L) in the set of symbols (S) is encoded by a nucleic acid sequence, and the codeword is decoded by ATD PCR and sequencing.

In the UniKey-Tag 1 system shown in FIG. 8 (a), the sequence of nucleotides in the variable region v is used to encode the string n, which is decoded by sequencing. For the template strand, the fragment length k=100 bp is comprised of a capping region and the 5′ and 3′ ends (Cp, 0-3 bp), a universal forward primer site (UPF, 10-30 bp), a complementary universal reverse primer site (UPR_{c}, 10-30 bp), and a variable region (V_x, 20-160 bp). The letters in the codeword are comprised of the set of nucleic acids {A, T, C, G, U} although more commonly the set {A, C, G, T} would be used (ie., U is not present in DNA). The length of each symbol in the codeword is l=1 to v bp. In the example shown in FIG. 8 (a), v=27 bp and l=3 bp allowing a codeword of length n=9 letters.

The codeword may be comprised of a string of alphanumeric or special character symbols that is used to lookup information associated with a product, item, or object. This information may include the product type, date of manufacture, date of expiry, manufacturing facility, and batch number for example. The encoding system used is dependent on the trade-off between decoding reliability and information density. In the most simple form each nucleotide is used to encode a letter L directly where l=1 bp and n=v according to the equation:

n_{UK1}=v (for direct encoding)

The maximum dataset of possible unique codewords for each primer pair is given by the equation:

$${w}_{*} = {\sum }_{n = 1}^{n = {n}_{m ax}} {s}^{n}$$

which is essentially limitless for all practical purposes. For example, considering only the case where s=4 and n=40, W₄₀=4⁴⁰≈10²⁴ unique taggant words. For context, this number is sufficient to provide every person in the world with more than 100,000 billion unique taggants. Note that denotes the set of taggants with different variable region lengths.

Sequencing and synthesis errors, however, mean that it may not be desirable to encode each letter with a single nucleotide. Building in controlled redundancy and error-correcting capabilities may be necessary to increase decoding reliability. As previously stated, reliability comes at a trade-off with data density. Although the design of DNA encoding systems with controlled redundancy is beyond the scope of the invention disclosure here, different systems have been developed for digital archival storage applications. These encoding systems contain data bits that encode information and parity bits that allow error detection and correction capabilities. Examples of systems with controlled redundancy include Hamming, Huffman, Reed-Solomon, Levenshtein, differential, single parity check, Goldman and XOR code¹⁻⁸. In Series 2 experiments, for example, Hamming (8,4,4) encoded symbols were used. These symbols contain l=8 nucleotides of which four are data bits and four are parity bits, giving a redundancy of 50%. The four data bits of quaternary code permit the generation of s=4⁴=256 different symbols. For codewords of length n=6 symbols, the total number of different codewords that can be generated (ie. taggant library size) with Ham(8,4,4) quaternary code is w=256⁶=2.8×10¹⁴.

The use of universal primer sequences for the set of codewords W_{*} means that any subset of fragments within W_{*} (Wᵤ ⊆W_{*)} are screened in one reaction according to the equation:

r_{UK1}=1

As such, for the UniKey-Tag 1 system, the number of screening reactions required is independent of both n and s.

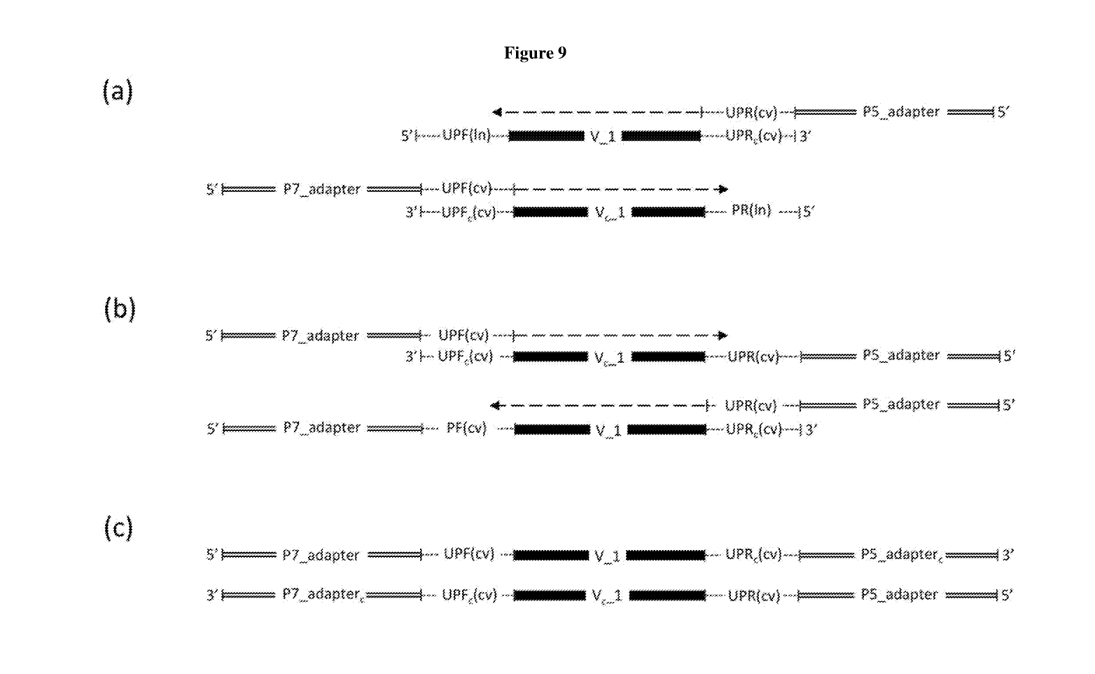

For UniKey-Tag 1, the ATD PCR products may be sequenced by Sanger sequencing, next generation ‘sequencing by synthesis’, or portable nanopore technology. Sequencing short amplification products is performed routinely using the Illumina platform and was also demonstrated in the Series 1 and 2 experiments using nanopore technology (ie. the Oxford Nanopore platform). For sequencing by synthesis, the incorporation of LNAs into the amplified product during ATD PCR does not present any issues since the process of adapter sequence ligation by PCR (required by sequencing by synthesis technologies) eliminates these LNAs from the prepared sample (FIG. 9). This leaves only conventional nucleotides in the products prepared for sequencing. No compatibility issues are therefore anticipated between UniKey-Tag recovery and amplification and existing sequencing technologies.

In the case of nanopore sequencing, the incorporation of LNAs into the samples during ATD PCR was not observed to contribute to sequencing error in the Series 1 and Series 2 studies disclosed here.

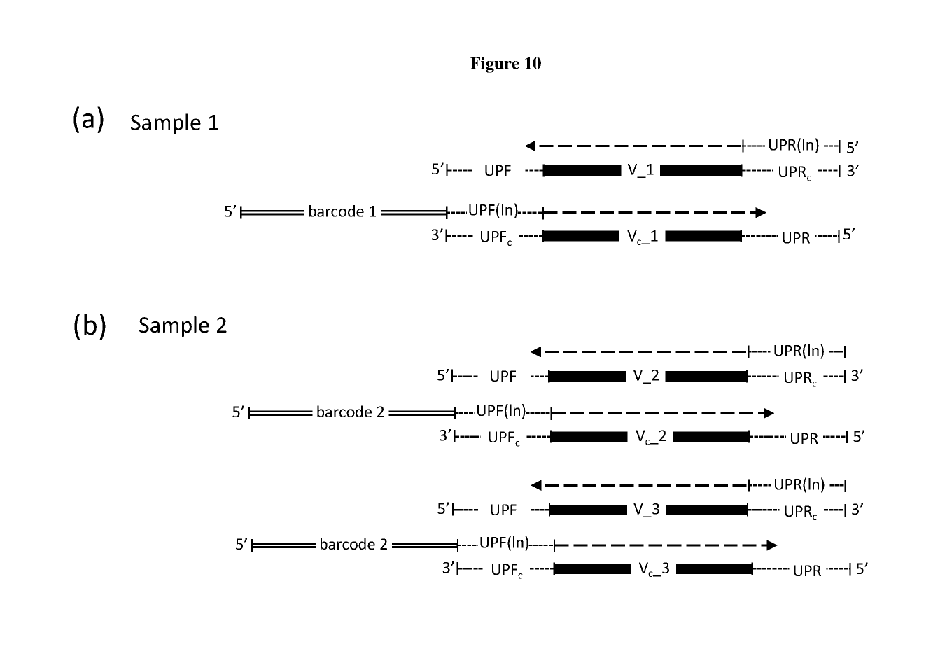

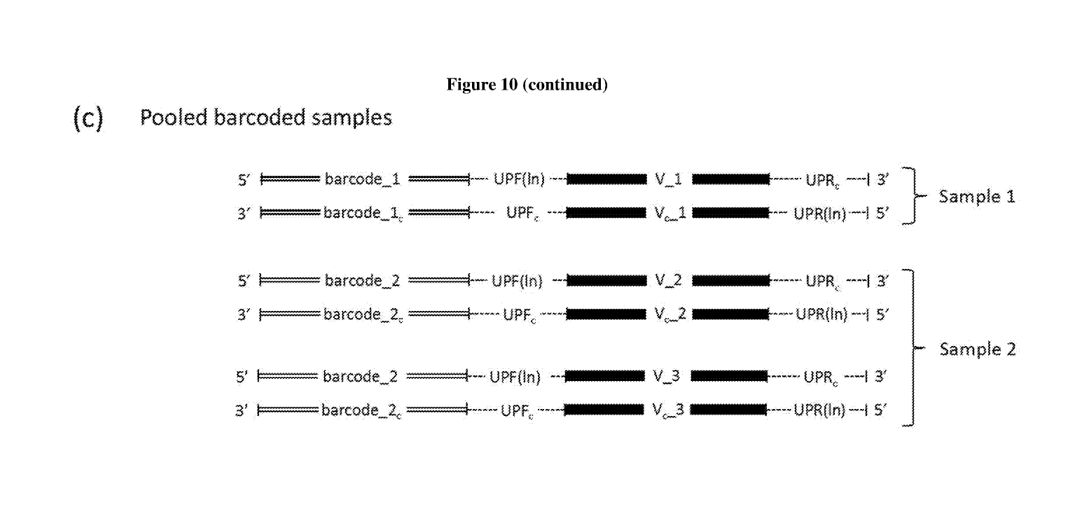

FIG. 10 shows that multiple samples may be sequenced and decoded in together by incorporating a barcode sequence that identifies a particular sample to the 5′end of the LNA primers used in ATD PCR. This allows multiple samples to be pooled together and sequenced in parallel, thereby improving sampling, sequencing and decoding efficiency. In the case of the UniKey-Tag 1 system, a universal set of primer pairs with a unique 5′ barcode identifier sequence may be used to sequence and decode multiple product samples, in parallel, for a particular industry (for e.g. pharmaceuticals).

Some of the advantages of the UniKey-Tag 1 system include:

1. Suitable for both identification and authentication purposes;

2. Billions of unique sequences available for each primer pair;

3. Layerable in the billions; and

4. High decoding efficiency

### Example 4—Design of Fragment Size-Encoded Taggants (UniKey-Tag 2 Taggants)

In the UniKey-Tag 2 system in FIG. 8(b) and FIG. 11 (fragment length encoded), each L in the set S is encoded by a full-length taggant and n is decoded by ATD PCR and fragment length separation.

In the UniKey-Tag 2 system shown in FIG. 8(b) and FIG. 11, each taggant encodes a symbol and the position of that symbol in a codeword string. A unique primer pair is assigned to each L in the set S which is used to identify the symbol type, and the length of each taggant τₛ determines the position of the symbol in the codeword string. The size of the set L is the codeword length and is determined by v divided by the resolution limit of gel electrophoresis (fᵣ) according to the equation:

$${n}_{UK 2} = \frac{v}{{f}_{r}}$$

Given that the maximum fragment size resolution of polyacrylamide gel electrophoresis is about 2 bp, and assuming a maximum taggant length of 100 bp and forward and reverse primer lengths of 20 bp, then n_{UK2} (max.)=60/2=30. Similarly, the maximum number of different positions that any particular symbol can occupy is 30.

In the UniKey-Tag 2 system, encoding is performed by fragment size separation (e.g. by gel electrophoresis), where the presence of a product for a particular primer pair indicates the symbol type, and the size of each product band (i.e. migration distance) determines the position of each letter in the codeword, according to the equation:

r_{UK2}=s

The number of amplification screening reactions required for the UniKey-Tag 2 system is equal to the alphabet size, r_{UK2}=S. This is because each set L is identified with a pair of LNA primers that is unique to that particular letter, and is amplified without cross-fragment hybridisation in one reaction using ATD PCR. As such each additional letter increases the layering depth in increments of 30 and requires only one additional screening reaction to decode.

For the n8-s3 UniKey-Tag 2 example given in FIG. 11 (a), S={A, B, C} where L_{A}={τ_{A1}, τ_{A2}, τ_{A3}}, L_{B}={τ_{B1}, τ_{B2}, τ_{B3}} and L_{c}={τ_{C1}, τ_{C2}, τ_{C3}, τ_{C4}, τ_{C5}}. FIG. 11 (b) shows how these taggants may be used to mark a product. First, each precursor is marked with a particular taggant τ so that the intermediate product contains layered taggants of the same letter-set L. The intermediate products are combined to form a final product that contains layered taggants that are members of the alphabet set S. In this example, taggant layering is performed at two levels, L and S. As the set of S contains only three different symbols, only three reactions are required to decode all of the taggants.

FIG. 11 (c) shows that the UniKey-Tag 2 taggants are decoded by simply recording the column (letter) and row (position) of each band on the electrophoresis gel. For example, the variable length taggants in FIG. 11 (a) would produce bands shown in the electrophoresis gel diagram in FIG. 11 (c), which are easily decoded as the n8-s3 codeword L_{A}={1A, 6A, 7A}; L_{B}={2B, 4B, 6B}; L_{c}={2C, 3C, 5C, 6C, 8C}. Note that it is permissible for the positions of different letters to overlap, i.e. {6A, 6B, 6C}. Taggant system 2 is most suited to product authentication applications where low level taggant layering is required and sequencing is not available. Missing letters suggest that a particular precursor is either absent or the product is counterfeit. The precursor may then be retested directly to determine authenticity.

It should also be noted that the UniKey-Tag 2 system shown in FIG. 11 effectively generates a two dimensional codeword (ie. two different letters can occupy the sample position on a gel) which permits massively expanded identification capacity. For example, an alphabet of size s=3 letters and a gel resolution limit that allows 30 different bands can generate 3×2³⁰=3.2×10⁹ different 2D electrophoresis gel images that may be used to identify a product.

Some advantages of the UniKey-Tag 2 system over prior art such as U.S. Pat. No. 8,735,327 include:
1. Conducive to low-copy number recovery: the number of samples required=s;
2. Deeply layerable: Each symbol in the alphabet S encodes a one-dimensional codeword of length n=30 (based on the presence or absence of a band). When combined with other letters the two-dimensional electrophoresis gel effectively forms a 2D ‘codeword’ which allows for massively expanded layering capacity.
3. Efficient to decode: only one ATD PCR reaction is required to amplify each symbol L, and only one electrophoresis gel lane per symbol is required decode the two dimensional ‘codeword’.

### Example 5—Annealing Temperature Discrimination (ATD) Polymerase Chain Reaction (PCR)

The ATD PCR protocol eliminates cross-hybridisation by artificially elevating the annealing temperature of the primers by incorporating locked nucleic acid (LNA) monomers into universal forward (UFP) and reverse (URP) primers. Therefore, the PCR annealing temperature may be set to a temperature that facilitates the formation of LNA primer-fragment complexes, but discriminates against cross-hybridisation that can occur at lower temperature.

LNA-primers were designed using the online tool provided by Exiqon so that the annealing temperature of the LNA-primers was at least 5° C. higher than the same conventional primer sequences that do not contain LNA monomers. The self-dimer (UFP-UFP and URP-URP) and hetero-dimer (UFP-URP) melting temperatures of the LNA primers was designed to be at least 30° C. below the LNA-primer annealing temperature. Here, UFP and URP are universal forward primer and universal reverse primer, respectively.

Amplification was performed by direct PCR (Thermo Scientific Phire Animal Tissue Direct PCR Kit, F-140WH) which was optimised to accommodate LNA-primers, low copy number taggant recovery and short fragment length visualisations using polyacrylamide gel electrophoresis.

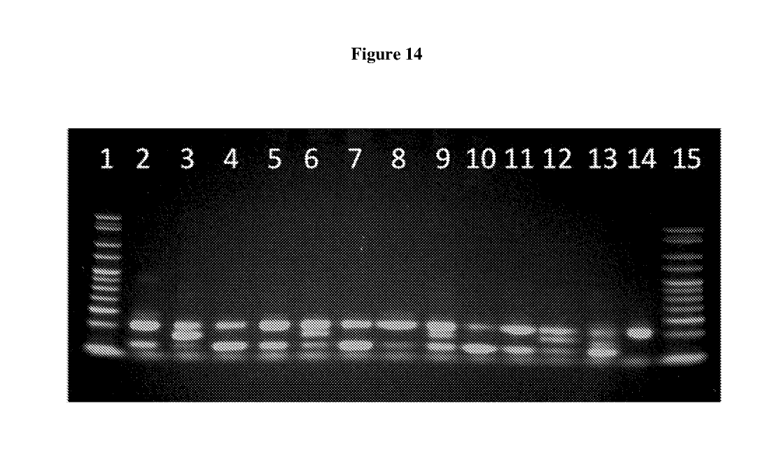

The effectiveness of PCR annealing temperature discrimination is illustrated in FIGS. 12-14 by using the oligonucleotide taggants OligoTag_1-20_Ser1 in Table 1. The photographs show PCR amplification products that are separated by fragment size using polyacrylamide gel electrophoresis. Clear distinct bands indicate individual fragment replication, whereas striations and smears indicate the presence of variable length products and cross-fragment hybridisation. For example, FIG. 12(a) shows no evidence of cross-taggant hybridisation over an annealing temperature (AT) range of 65-69° C. (design AT of 69° C.), but FIG. 12(b) shows extensive taggant-taggant hybridisation for the equivalent experiment using conventional primers over the annealing temperature range 49-53° C. (design AT of 53° C.). FIG. 13 demonstrates that ATD PCR prevents hybridisation of variable length fragments and under varying thermal cycle time intervals. Lastly, FIG. 14 confirms the validity of ATD PCR in the field for the application of ammunition tracing (see also Example 6).

### Example 6—UniKey-Tag Technology for Tracing Firearms Crime Via Ammunition Dispersed Taggants (Identification)

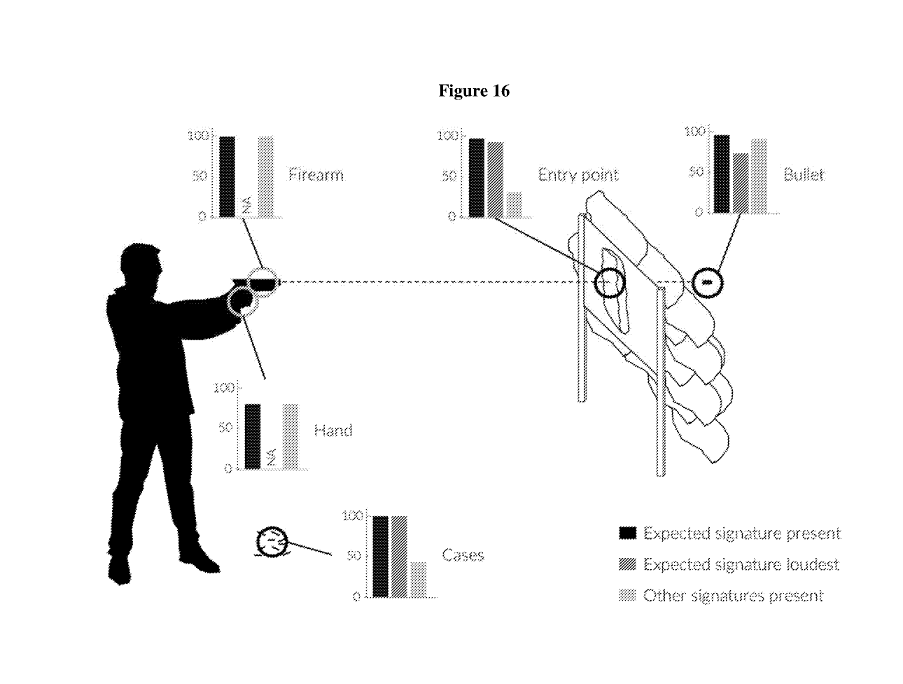

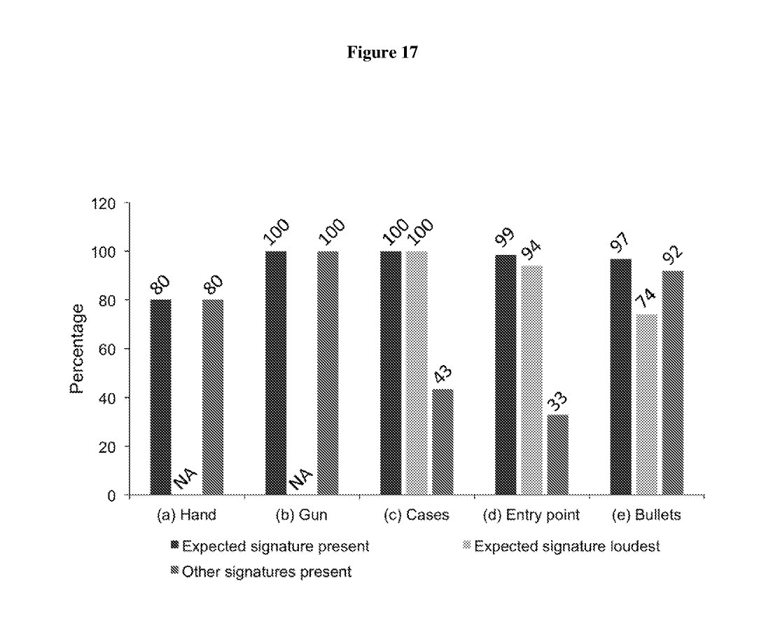

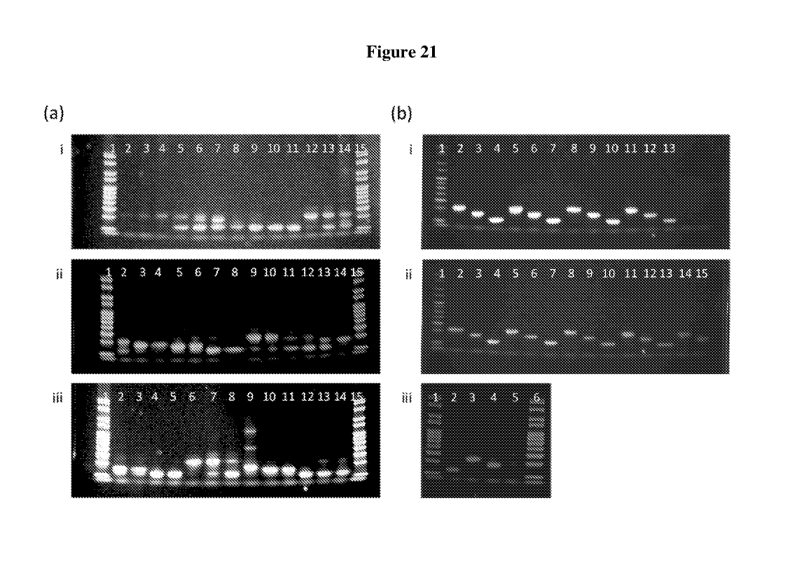

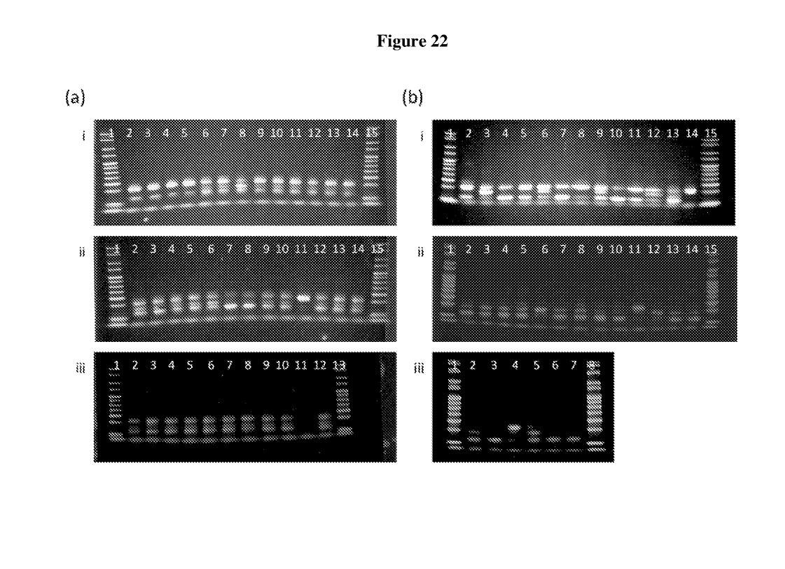

The UniKey-Tag system was used to encode identification information into synthetic oligonucleotides that were subsequently preserved in a fixing solution and deposited onto the surface of ammunition cartridges. Fixing agents were screened to protect against high temperature, high pressure, ultraviolet radiation (UV) and nuclease activity without inhibiting downstream enzymatic processes required for taggant amplification. The UniKey-Tag systems were tested using a .22 caliber firearm (impact energy, Eᵢ=420J), Browning 9 mm handgun (Eᵢ=470J) and .270 caliber firearm (Eᵢ=3,660J). The 9 mm handgun was chosen because similar guns were used in 5,562 homicides in the United States in 2014, representing 68% of gun-related- and 47% of total homicides, respectively. The .270 firearm was chosen to demonstrate our protocol in equivalent high impact energy assault rifles used in military applications. Labeled ammunition was fired at targets comprised of ventral sections of Sus scrofa domesticus (supermarket pork belly) as an analogue for human tissue, and fragment recovery was tested at five points: the hand of the shooter, firearm, cartridge cases, bullet entry point, and recovered bullet. Results show that an unbroken chain of identification was established in almost all trials for all firearms (See FIGS. 16, 17, 19-22) linking the labelled ammunition to the shooter, firearm, cases, recovered bullets and bullet entry point, with any one of these five recovery points sufficient for suspect identification. This technology has clear applications for tracing illegal and black market arms transfers, detecting arms embargo violations, exposing weaknesses in stockpile management, tracing 3D-printed and modular weapons, and identifying groups involved in the illegal wildlife trade.

The advent of modular, polymer, and 3-dimensional (3D) printed guns has brought new challenges for firearms tracing and registration. Full modularity allows users to reconfigure firearms from parts of the same or related models to meet different operational needs, including changing the calibre of the weapon. Light-weight polymer framed firearms are difficult to mark with tamperproof serial numbers and post-manufacture import stamps, and may evade detection by conventional screening technologies. Whilst advances in 3D printing offer clear benefits for professional firearms manufacturers, there is considerable anxiety that this technology could soon allow individuals and criminal organisations to fabricate firearms at home. Despite the recent introduction of legislation to restrict or ban the sale of 3D guns in some countries, both guns and plans are freely available at online illegal marketplaces and file sharing websites.

Concerns that firearms registration and tracing capabilities are lagging behind advances in firearms technology has been highlighted in numerous reports and by initiatives that monitor the international arms and ammunition trade. One way to address these challenges is to mark ammunition with an identifiable molecular ‘barcode’ that is dispersed upon use. Such a system would ideally leave an unbroken molecular signature on the firearm, user, bullet and victim or target, as well as provide a history of the ammunition previously used in the firearm. The registration of civilian and law enforcement agency ammunition could aid forensic investigations and provide a strong deterrent to gun-related crime. Tagged ammunition could also offer the capacity to trace illegal and black market arms transfers, detect arms embargo violations, expose weaknesses in stockpile management, and identify groups involved in the illegal trade of wildlife. Until now, the lack of attention to ammunition tagging technologies could have been due to a perceived lack of application (before the advent of modular and 3D printed firearms), barriers to market entry (most notably the requirement for policy intervention) or perceived technical barriers that for the most part no longer exist (after several recent advances in synthetic nucleic acid technology and DNA sequencing).

In this experiment, UniKey-Tag technology is demonstrated for tracing firearms and firearms crime using tagged ammunition. The underlying concept of ammunition fingerprinting is that because ammunition is in contact with the shooter, firearm and victim, it provides the best means of transferring information to a crime scene. Each UniKey-taggant contains a variable encoding region that is flanked by universal forward and reverse primer sequences. This allows an essentially unlimited number (1,000's billions) of taggants to be screened in one amplification reaction. Existing taggant technologies are unsuitable for large-scale identification and deep layering applications, such as ammunition tracing.

The UniKey-Tag system also meets other design criteria that are required for ammunition tracing and supply chain monitoring of consumable products:
Suitable for identification and authentication purposes (see precise definitions given in the terms and abbreviations);
Of broad scope: The capacity to generate, recover, and decode billions of unique identifier sequences cheaply and efficiently;
Highly covert: Must be invisible and undetectable without prior knowledge of ‘chemical keys’ (primer sequences);
Non-toxic: is safe for human consumption and/or entry to the blood stream (in small amounts);
Resistant: Must be resistant to high temperature, high pressure, UV radiation, nuclease exposure and tamper proof;
Dispersed by contact (tagged ammunition must ideally leave a traceable signature on the gun, user, victim/target and bullet);
Recoverable in very low copy-number after dispersal;
Inexpensive: must represent a small fraction of the value of the product (ie. <1% of the cost), and sufficiently inexpensive to achieve full market penetration to serve as an effective deterrent;
Easily integrated into existing ammunition production processes (ideally as a post manufacturing step);
Nano-scale: Tags deposited on the surface must remain within the safety tolerance limits as defined by the manufacturer;
Environmentally safe and non-toxic.

The goal of this experiment was to test UniKey-Tag technology in the field as an ammunition dispersed/transferred taggant. The methodologies described here exploit several recent advances in nucleic acid technology in combination with novel protocols designed to reduce oligonucleotide degradation and aid low copy number tag recovery. Methodologies were optimised for taggant dispersal from ammunition cartridges to the firearm, user, and target or victim, with the aim to provide better forensic capabilities to trace gun-related crime. The protocols were tested using low and high muzzle energy (ME) firearms, including a .22 calibre rifle (ME=420J), a nine millimetre Browning handgun (ME=470J), and a .270 calibre rifle (ME=3,660 J). Taggant recovery was tested at five points: the firearm, cartridge casing, user, bullet, and entry wound (sections of pig tissue were used).

### Methodology

This experiment was structured in two main parts. First, an accelerated degradation study was performed to test the capacity of candidate fixing agents to protect taggants against high temperature, high pressure, ultraviolet radiation (UV) and nuclease activity. These fixing agents were also screened to ensure against possible inhibitory effects on downstream enzymatic processes required for taggant recovery and amplification.

In the second part of the experiment, ammunition cartridges were marked with taggants suspended in selected fixing solution and fired at a target. Taggant recovery was tested at the following five points: hand of the shooter, firearm, ammunition cases, bullet entry point, and recovered bullet.

### (A) UniKey-Tag Design and Preparation

Two different taggant encoding systems were tested in Series 1 and Series 2 experiments. In Series 1 experiments (9 mm handgun ammunition tracing) the taggants given in Table 1 were used. These taggants were designed in accordance with UniKey-Tag 1 and 2. Specifically, the taggants were designed with 5′ and 3′ end capping regions (0-3 bp), universal forward and reverse primer sites (20 bp), and a variable length codeword region (10-40 bp). In total, 40 complementary ssDNA oligonucleotides were ordered and subsequently annealed to form 20 dsDNA taggant duplexes. These 20 taggants included four of each of the following lengths: 80, 76, 70, 66, and 60 bp so that identification could be performed by fragment length separation in accordance with the UniKey-Tag 2 system as well as sequencing in accordance with the UniKey-Tag 1 system. The design specifications and sequences of these 20 taggants are given in Example 1 and Table 1. All of these taggants were designed with identical forward and reverse primer sites.

In Series 2 experiments (.22 and .207 calibre firearm ammunition tracing and pharmaceuticals labelling) taggants were similarly designed in accordance with UniKey-Tag 1: taggants were designed with 5′ and 3′ end capping regions (0-3 bp), universal forward and reverse primer sites (22 bp), and variable encoding region of variable length (46 bp). One difference compared to Series 1 experiments, but still consistent with the UniKey-Tag 1 system, is that the variable region was encoded with six Hamming(8,4,4) crumbs selected from a library of 256 crumbs (See Table 4 and FIG. 15). In Series 2 experiments, decoding was performed by sequencing only.

Taggants were synthesised by Sigma-Aldrich and purified using high-performance liquid chromatography (HPLC). Production was performed at several different locations and distributed over several weeks to ensure against cross-contamination.

### (B) Single-Strand Duplexing

Single-stranded oligonucleotide templates were re-suspended in 400 μL of 10 mM Tris-EDTA (10 mM Tris, 50 mM NaCl, 1 mM EDTA, pH 7.5-8.0, Sigma 93284), vortexed for 10 seconds and, optionally, centrifuged for 1 minute. The re-suspended template strands were transferred into the tubes containing the respective complementary single-stranded taggant. This process was repeated two more times for Tris-EDTA aliquots of 400 μL and 200 μL, bringing the combined template-complementary strand solution to 1000 μL. The solution was placed on a heat block at 95° C. for 5 minutes then ramp-cooled to 25° C. over a period of one hour to facilitate duplex formation. The dsDNA taggants were stored at −20° C. for further use.

### (C) Universal Primer Design Annealing Temperature Discrimination (ATD) Polymerase Chain Reaction

The annealing temperature discrimination (ATD) polymerase chain reaction (PCR) methodology performed in these experiments was designed such that primer-fragment interactions occur at an annealing temperature that is least 5° C. above the annealing temperature of fragment-fragment interactions (e.g. Δ_{AT} 5° C.). This was achieved by incorporating locked nucleic acids (LNA) into the universal forward (UFP) and reverse primers (URP) used for taggant recovery and amplification. LNA-primers were designed using the online tool provided by Exiqon so that Δ_{AT} 5° C.; and self-dimer (UFP-UFP and URP-URP) and hetero-dimer (UFP-URP) melting temperatures were at least 30° C. below the LNA-primer-fragment binding temperature.

The design specifications of the universal set of LNA-primers used in Series 1 and 2 experiments are given in Tables 5 and 6, respectively. Locked nucleic acids are preceded by the symbol ‘+’. The annealing properties of the equivalent set of conventional primers are also given for comparison.

TABLE 5
Primer sequences and annealing properties used in Series 1
experiments (9 mm handgun ammunition tracing)

| | | | | | |
|---|---|---|---|---|---|
| | | | | Self | Hetero- |
| | Length | GC | Tm | dimer | dimer |
| Sequence (5′ → 3′) | (bp) | (%) | (° C.) | (° C.) | (° C.) |

Universal primers (9 mm experiments)

| | | | | | | |
|---|---|---|---|---|---|---|
| For. | ATC + TG + ACTT + TC + G + | 20 | 40 | 69 | 33 | 31 |
| SEQ ID NO: 61 | TAT + C + TAGC | | | | | |
| Rev. | TGTG + TATG + AAC + T | 20 | 40 | 69 | 36 | |
| SEQ ID NO: 62 | TC + TC + AT + GG | | | | | |

Equivalent conventional primers

| | | | | | | |
|---|---|---|---|---|---|---|
| For. | ATCTGACTTTCGTAT | 20 | 40 | 53 | 17 | 24 |
| SEQ ID NO: 63 | CTAGC | | | | | |
| Rev. | TGTGTATGAACTTCT | 20 | 40 | 56 | 19 | |
| SEQ ID NO: 64 | CATGG | | | | | |

The symbol ‘+’ precedes a locked nucleic acid (LNA).

TABLE 6
Primer sequences and annealing properties used in Series 2
experiments (0.22 and 0.207 calibre firearm ammunition
tracing and pharmaceuticals labelling)

| | | | | | |
|---|---|---|---|---|---|
| | | | | Self | Hetero- |
| | Length | GC | Tm | dimer | dimer |
| Sequence (5′ → 3′) | (bp) | (%) | (° C.) | (° C.) | (° C.) |

Universal primers (9 mm experiments)

| | | | | | | |
|---|---|---|---|---|---|---|
| For. | TTTC + TGT + T + GGTGC | 22 | 41 | 74 | 15 | 21 |
| SEQ ID NO: 65 | TG ATATTGC | | | | | |
| Rev. | ACTTG + CCTG + T + CG | 22 | 50 | 74 | 28 | |
| SEQ ID NO: 66 | CTCTATCTTC | | | | | |

Equivalent conventional primers

| | | | | | | |
|---|---|---|---|---|---|---|
| For. | TTTCTGTTGGTGCTG | 22 | 41 | 54 | 30 | 29 |
| SEQ ID NO: 67 | ATATTGC | | | | | |
| Rev. | ACTTGCCTGTCGCTC | 22 | 50 | 57 | 16 | |
| SEQ ID NO: 68 | TATCTTC | | | | | |

The symbol ‘+’ precedes a locked nucleic acid (LNA).

### (D) Annealing Temperature Discrimination PCR Protocol

Taggant amplification was performed using established direct polymerase chain reaction (PCR) methodologies (Thermo Scientific Phire Animal Tissue Direct PCR Kit, F-140WH) with further refinements to accommodate LNA containing primers, low copy number taggant recovery, and short fragment length visualisation using polyacrylamide gel electrophoresis. Direct PCR was used to bypass additional purification steps that could result in sample loss.

The PCR reagents used in Series 1 and 2 experiments are given in Table 7 and the thermal cycle protocols are given in Table 8 and 9. The thermal cycle annealing temperature was set to 67° C. (Δ_{AT}≥16° C.) in Series 1 experiments (Table 8) and 70° C. (Δ_{AT}≥16° C.) in Series 2 experiments (Table 9) to ensure against cross-taggant priming and hybridisation. Note that a higher concentration of primers and greater number of thermal cycles compared to standard protocols⁹ were require to produce sufficient short-length product (post amplification length of 54-80 bp) for sequencing and to distinguish and decode bands by fragment length separation gel electrophoresis.

PCR products were resolved by fragment size using polyacrylamide gel (12%) electrophoresis. The gels were stained with ethidium bromide and inspected under high UV. Selected bands were excised for Sanger sequencing.

TABLE 7
PCR reagents for Series 1 and Series 2 experiments

| | μL per reaction |
|---|---|
| PCR buffer 2x | 25 |
| Primer universal forward | 2 (of 50 uM) |
| Primer universal reverse | 2 (of 50 uM) |
| Polymerase (Phire Hot Start II) | 1 |
| H₂O | 15 |
| Sample | 5 |
| Total | 50 |

TABLE 8
PCR thermal cycle protocol - Series 1 experiments

| Cycle details | Time (s) | Temperature (° C.) | | Cycles (no.) |
|---|---|---|---|---|
| Initial denaturation (a) | 300 | 98 | | 1 |
| Denaturation (b) | 5 | 98 | | |
| Annealing (c1, c2) | 5 | 67 | (LNA primers) | 50 |
| | | 51 | (Conv. primers) | |

| | | | |
|---|---|---|---|
| Extension (d) | 20 | 72 | |
| Final extension (e) | 60 | 72 | 1 |
| Storage (f) | Forever | 4 | 1 |

** Letters (a-f) correspond to PCR phases in FIG. 6.

TABLE 9
PCR thermal cycle protocol - Series 2 experiments

| Cycle details | Time (s) | Temperature (° C.) | | Cycles (no.) |
|---|---|---|---|---|
| Initial denaturation (a) | 300 | 98 | | 1 |
| Denaturation (b) | 5 | 98 | | |
| Annealing (c1, c2) | 5 | 70 | (LNA primers) | 50 |
| | | 54 | (Conv. primers) | |

| | | | |
|---|---|---|---|
| Extension (d) | 10 | 72 | |
| Final extension (e) | 60 | 72 | 1 |
| Storage (f) | Forever | 4 | 1 |

** Letters (a-f) correspond to PCR phases in FIG. 6.

### (E) Fixing Solution Screening: Accelerated Degradation Experiment Under High Temperature and High Ultra Violet (UV) Light

A list of candidate fixing solutions were identified and screened for their capacity to protect taggants against high temperature, high pressure, ultraviolet radiation (UV) and nuclease activity. The fixing agents were also required to function as a physical adherent and have no or low inhibitory effects on downstream enzymatic processes required for fragment recovery and amplification using direct ATD PCR.

The fixing solutions given in Table 10, below, include: 0.1, 0.3 and 0.6 M solutions of D-(+)-trehalose dihydrate (Sigma 90210), 0.1 M solution of α,β-trehalose (Sigma T0299), and 1% m/m solution of polyvinyl alcohol (Sigma 360627) dissolved in 10 mM Tris-EDTA (Sigma 93284). Each solution was prepared to contain 0.8 μM dsDNA of OligoTag_1_Ser1 (See Example 1). The control solution was 100% 10 mM Tris-EDTA.

TABLE 10
Oligonucleotide fixing solution suspensions

| | | Molarity | Molarity |
|---|---|---|---|
| | | fixing agent | dsDNA Tag |
| Solution | Notes | (M) | (μM) |
| C1 | Control, 10 mM Tris-EDTA | None | 0.8 |
| T1 | D-(+)-trehalose dihydrate | 0.1 | 0.8 |
| T2 | D-(+)-trehalose dihydrate | 0.3 | 0.8 |
| T3 | D-(+)-trehalose dihydrate | 0.6 | 0.8 |
| Tab | α,β-trehalose | 0.1 | 0.8 |
| PVA | Polyvinyl alcohol | 1% m/m | 0.8 |

The taggant solutions C1, T1, T2, T3, Tab, and PVA (Table 10) were deposited onto 8×12 mm brass plates using an airbrush gun. The deposited layer was less than 50 μm thick, which is well inside the design tolerances of ammunition cartridges. Brass plates were used to simulate the surface of ammunition cartridges. The fixed taggants were exposed to continuous high light (UVA and UVB, 1,000 μmol m⁻² s⁻¹) and high temperature (50° C.) conditions over a four-month period. Taggants were recovered from the plates at day 5, 8, 13, 21, 34, 55 (n=3 per recovery cycle for each fixing solution) and tested for the amount of dsDNA present and taggant amplification viability.

Taggants were recovered from the brass plates by immersing in 500 μL 10 mM Tris-EDTA buffer, heated to 50° C. for 3-4 minutes and vortexed. This step was repeated three times before the brass plates were removed. A 5 μL aliquot of the remaining solution was introduced directly into PCR wells containing a pre-prepared reagents.

The amount of dsDNA remaining on the plates at each time interval was quantified using Qubit fluorometric quantification methodology (Invitrogen, Q32854). To ensure against artefactual readings, the reference sample for each solution contained only the fixing agent suspended in 10 mM Tris-EDTA.

### (F) Ammunition Tagging for Firearms Tracing

Oligonucleotide taggants were suspended in a fixing solution and deposited onto the surface of the .22 caliber, 9 mm and .207 caliber ammunition cartridges. For the Series 1 experiments, four cartridges were marked with each of the 20 taggants given in Example 1. For the Series 2 experiments five .22 caliber and four .207 caliber ammunition cartridges were each marked with each of the 10 taggants given in Table 3. The marked ammunition was fired at a target comprised of a section of pig tissue (supermarket pork belly) from a distance of 15 m. The pig tissue was used as an analog for human tissue and to simulate conditions that may contribute to nuclease-mediated taggant degradation. The target was placed in front of sandbags to facilitate bullet recovery. Taggant recovery was tested at the five points shown in FIGS. 16, 17 and 19. These five points are the: (a) hand of the shooter, (b) firearm, (c) ammunition casing, (d) bullet entry point, and (e) recovered bullet.

### (G) Taggant Recovery:

Three taggant recovery protocols were developed for the substrate classes: (1) soft tissue, (2) hard surfaces and skin, and (3) fragmented material. These protocols were designed to avoid excessive handling (optimised for low copy number tag recovery), to be compatible with direct PCR methodologies, and to optimise taggant recovery from the five-point recovery locations.

Protocol 1: Soft Tissue: Tag Recovery from the Entry Wound.

Two methods of tag recovery from the entry wound were tested: (1) a refined version of the wet swab-dry swab methodology as previous described by Williams et al. (2013, Journal of Forensic Research, 4: 4-6) and (2) the excising of tissue from the entry wound for introduction into direct PCR protocols.

In the first method, buccal swabs (Isohelix, MS-001:1) were moistened with an aerosol of 0.1 mM Tris-EDTA. The swab was rotated around the bullet entry site taking care to make contact with the upper quarter of the swab head only. A second dry buccal swab was used to re-swab the site and surrounding tissue. Samples were placed on ice immediately and stored at −20° C.

To recover DNA taggants from the swab, swabs were re-moistened with an aerosol of 0.1 mM Tris-EDTA and the swab head was inserted into a 100 μL pipette tip to express the liquid. A 5 μL aliquot of the liquid that collected in the tip was introduced into wells containing direct PCR reagents given in Table 3. If PCR amplification failed, the swab tip was cut off and introduced directly into the wells containing direct PCR reagents as a ‘backup’. Dry swabs were tested if both wet swabs failed.

In the second approach, a small amount of tissue was excised from the bullet entry site and placed in a 2 mL Eppendorf tube. The tissue was suspended in 600 μL of 0.1 M Tris-EDTA solution and heated to 82° C. for two minutes and vortexed twice. A 5 μL aliquot of the liquid fraction (supernatant) was introduced into wells containing the direct PCR reagents given in Table 2.

Protocol 2: Hard Surfaces and Skin: Tag Recovery from the Firearm and User

A polyvinylalcohol-based gel was used to recovery taggants from the firearm and the hand of the shooter. The gel was prepared by dissolving PVA (10%) in ethanol (10%) and water at 70° C. for 3-4 hours (or until all PVA crystals dissolved). A thin film of the gel was applied to the sampling area, allowed to set, then peeled off and stored at −20° C.

To recover taggants from the PVA film, the film was dissolved in 0.1 mM Tris-EDTA at 60° C. for two minutes. Approximately 200 μL cm⁻¹ of Tris was typically sufficient to dissolve the film. A 5 μL aliquot of the resulting solution was introduced directly into wells containing PCR reagents.

### Protocol 3: Ammunition Casing and Bullet

Taggant recovery from fragmented material (such as bullet fragments or cartridge casing) was performed by immersion in Tris-EDTA buffer. The immersed material was heated to 50° C. for 2-3 minutes and vortexed briefly. This heating-vortex cycle was repeated three times before the casings or bullet fragments were removed from the solution and stored at −20° C. Note that metallic fragments should not be left in solution for more than 30 minutes as dissolved metal ions may inhibit downstream PCR reactions. For example, when brass cartridges were left in solution overnight, the suspension turned blue indicating a high concentration of dissolved copper ions.

### (H) Nanopore Sequencing Protocol

To recover taggants from the PVA film, the film was dissolved in 0.1 mM Tris-EDTA at 60° C. for two minutes. Approximately 200 μL cm⁻¹ of Tris was typically sufficient to dissolve the film. A 5 μL aliquot of the resulting solution was introduced directly into wells containing PCR reagents.

Samples were prepared and sequenced according to the 1D, 96 PCR barcoding protocol for amplicons (Oxford Nanopore protocol number: SQK-LSK108). PCR barcoding was performed according to this protocol with Phire HotStart II polymerase. In all UniKey-Tag 1 experiments sequencing was carried out using FLO-MIN106 (R9.4) flowcells and samples were decoded with the Needleman-Wunsch algorithm modified for semi-global sequence alignment.

### Results

### Accelerated Degradation of Preserved DNA Taggants

In the accelerated degradation experiment, taggants were suspended in the fixing solutions in Table 10 and deposited onto brass plates. The plates were exposed to sustained high temperature (50° C.) and electromagnetic radiation condition (ie. light including UVA and UVB, 1,000 μmol m⁻² s⁻¹) over a 55 day period. Polyvinyl alcohol was tested as a candidate fixing agent because it has previously been used in DNA storage protocols. Trehalose is also thought to protect against DNA damage in organisms that desiccate, and has also previously been tested as a DNA fixing agent for commercial purposes.

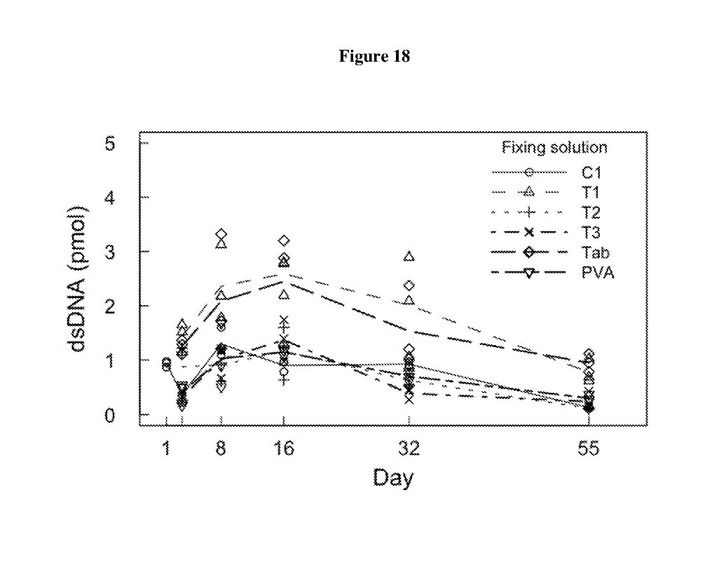

The results presented in FIG. 18 show the amount of DNA recovered at various time intervals over a 55 day period, as determined by Qubit fluorometric quantification. The amount of dsDNA recovered ranged from approximately 0.5-3.0 pmol plate⁻¹ and exhibited similar degradation rates of 0.03 pmol d⁻¹ for all fixing solutions. According to FIG. 18, the performance of fixing solutions at Day 55, in order of most to least dsDNA recovered was: Tab, T1, PVA, T3, T2, and C1. These data, however, do not show a conclusive difference between the capacity of the solutions tested to preserve fragments. Upon closer examination, the data show that the amount of dsDNA recovered from Tab and T1 solutions was higher than for PVA, T3, T2, and C1 solutions from Day 1, and remained proportionally higher over the duration of the experiment. Whilst dsDNA recovery was consistently highest for Tab and T1 solutions, no significant difference was observed between the rate of change of degradation between all the solutions tested, including the C1.

PCR products were successfully obtained from all fixing solutions, including the control, at each sampling time interval. In terms of DNA preservation and PCR compatibility, therefore, no significant differences were observed between the fixing solutions tested and the control. These results are testament to the durability of DNA.

The key finding of this accelerated degradation experiment is that both trehalose and polyvinyl alcohol do not exhibit significant inhibitory effects on Phire Hot Start II polymerase activity. Polyvinyl alcohol was the preferred fixing agent because D-(+)-trehalose dehydrate absorbed moisture from the atmosphere and formed a sticky layer on the plates, and α,β-trehalose was too expensive for practical applications (USD 16,300 g⁻¹).

Ammunition Fingerprinting with the UniKey-Tag 1 System (.22, .207 and 9 mm Firearms) and the UniKey-Tag2 System (9 mm Firearm Only): dsDNA Taggants are Dispersed onto and Recoverable from the User, Firearm, Casing, Entry Point and Bullet after Firing

In this section the results of ammunition tracing experiments with the UniKey-Tag 1 system are presented first. In these experiments the Series 1 taggants (Table 1) were used to mark the 9 mm ammunition cartridges and Series 2 taggants (Table 4) were used to mark the .22 and .207 caliber ammunition cartridges. In accordance with the UniKey-Tag 1 system, the samples were amplified by ATD PCR, sequenced (using nanopore technology), and decoded.

Second, results of the ammunition tracing experiments using the UniKey-Tag 2 systems are presented. These experiments were only conducted with the 9 mm handgun and used variable length Series 1 taggants only (given in Table 1). In accordance with UniKey-Tag 2, samples were amplified by ATD PCR and decoded by fragment length separation.

For both UniKey-Tag 1 and 2 experiments, samples were taken from the hand, firearm, used cases, bullet entry point, and recovered bullets.

(l) UniKey-Tag 1 Ammunition Fingerprinting Experiments with Series 1 Taggants (9 mm Handgun) and Series 2 Taggants (.22 and .207 Caliber Firearms): Decoding by Sequencing.

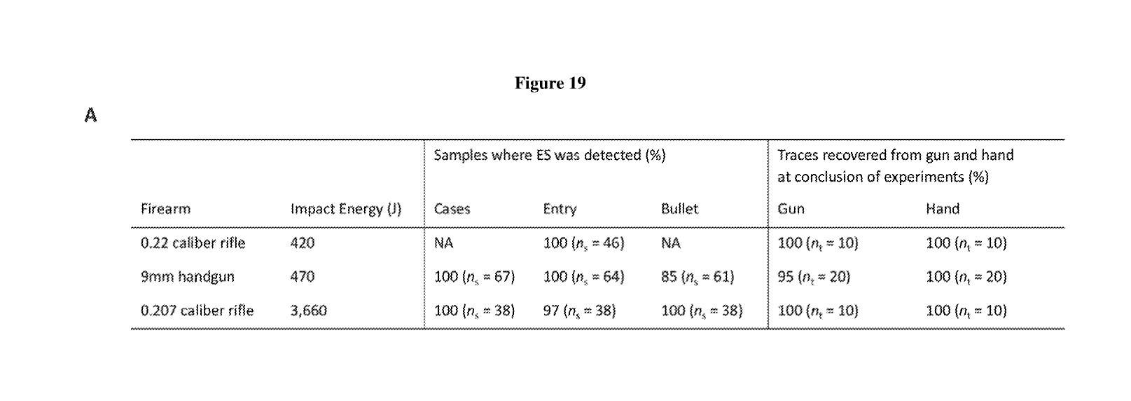

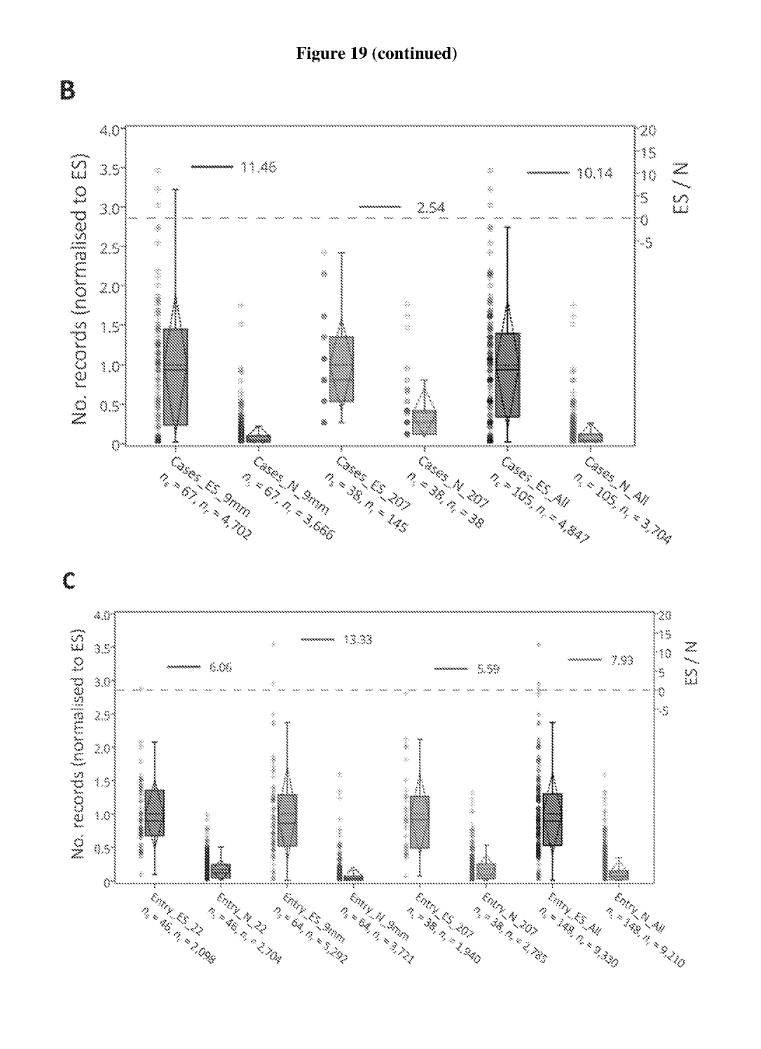

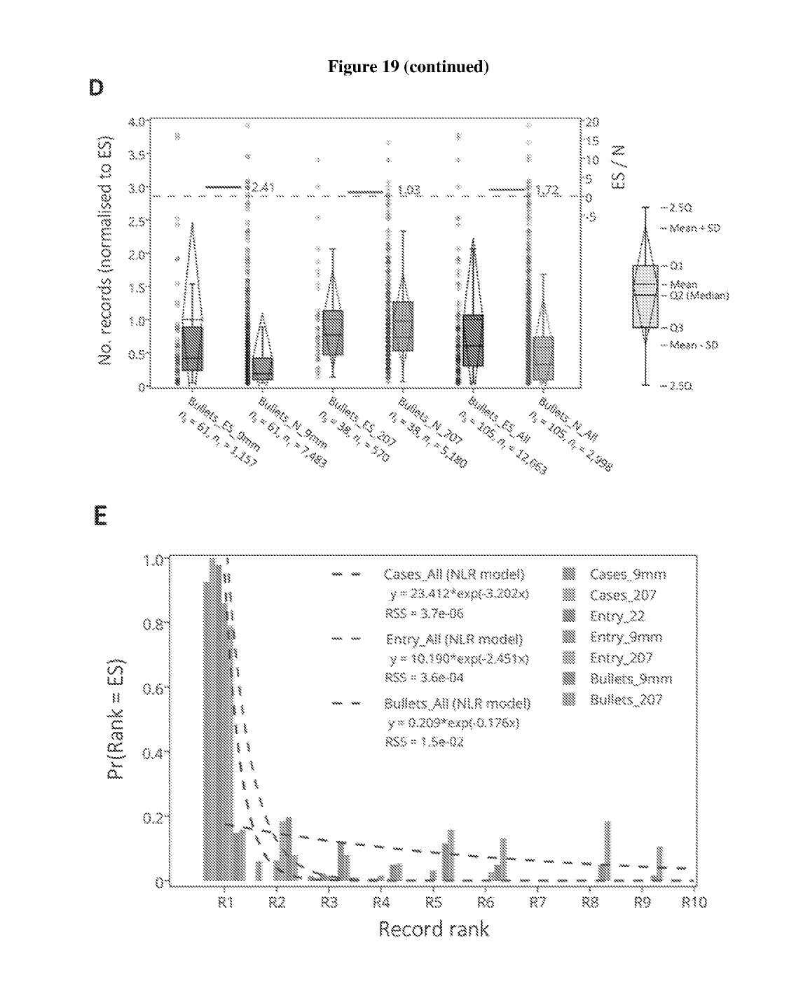

Results of the UniKey-Tag 1 experiments with the 9 mm handgun (Series 1 taggants) and .22 and .207 caliber firearm (Series 2 taggants) are given in FIG. 19 (a-e). In the UniKey-Tag 1 system, the DNA taggants are amplified by ADT PCR sequenced and decoded. In the case of the experiments described here, the samples were amplified using ATD PCR, barcoded with a sample identifier sequence, pooled together and sequenced using portable nanopore technology. The sequenced results were then decoded using the Needleman-Wunsch algorithm modified for semi-global sequence alignment.

Results of the ammunition tracing experiments are shown in FIG. 19(a-e). FIG. 19(a) shows the frequency that the expected DNA trace was detected, (b-d) shows expected signal (ES) and noise (N) records for case, entry and bullet samples respectively, and (e) shows the probability of correct identification as function of record rank. The ES and N metrics are the read count for each fragment record detected in a sample normalized to ES, and the rank is the read count for each record listed from highest to lowest. We chose this approach because the same firearm was used in all trials, which allowed fragment transfer between different sets of labelled ammunition loaded successively into the gun. This experimental design was used to reflect civilian gun use patterns and to test if we could probabilistically link the ES to rank. The results for Pr(Rank=ES) given in (E) include predictive non-linear regression (NLR) models for aggregated case, entry and bullet samples. The rank 1 (R1) value, for example, is the confidence that the highest ranked record, in a sample where multiple records are detected, correctly identifies the ammunition used in that particular trial.

FIG. 19 (a) shows that an unbroken chain of identification was detected in almost all case, entry point and bullet samples. Two exceptions were the .207 entry point (97%) and 9 mm bullet samples (85.2%). At the conclusion of the experiments, samples were recovered from the hand of the shooter and gun. In almost all hand and gun samples, each set of labelled ammunition was detected. The only exception was the 9 mm handgun, where 19 of the 20 sets of labelled ammunition was detected on the gun at the conclusion of the experiments (see FIG. 19(a)). The ES and N values for each trial are given in FIG. 19(b-d).

### Relationship Between Record Rank and Expected Signal

The relationship between the record rank and expected signal is given in FIG. 19 (e). For case samples, the rank 1 (R1) record correctly identified the ammunition cartridge used in a particular trial on 97% (n=67 trials) and 100% (n=100% trails) for the 9 mm and .207 caliber firearms, respectively. Samples from the cases were not taken in the .22 caliber firearms experiment.

For entry point samples, the probability that the R1 record correctly identified the ammunition cartridge used in a particular trial was 0.98 (nₛ=46) for the .22 firearm, 0.86 (nₛ=64) for the 9 mm handgun and 0.79 (nₛ=38) for the .207 firearm. In the case of recovered bullet samples, the probability that the R1 record correctly identified the ammunition cartridge used in a particular trial was 0.15 and 9 mm handgun and 0.16 for the .207 firearm. Bullet samples were not taken in the .22 caliber firearm experiments.

Aggregated across all firearms types, the probability that the correct cartridge was identified in rank 1-3 records (R1-R3), was 0.99 for case samples, 0.96 for entry point samples, and 0.44 for recovered bullet samples. These experiments demonstrate that synthetic DNA is a suitable media for labelling ammunition and tracing gun crime in both low and high muzzle energy (ME) firearms. The ME for the firearms tested ranged from 440 J (.22 caliber firearm) to 3,660 J (.207 caliber firearm).

The results showed that ammunition labelled with synthetic DNA leaves an unbroken chain of identification on the shooter, gun, cases, entry point, and recovered bullet after firing with any one of these points sufficient to determine the origin of the marked ammunition. The signal-to-noise approach demonstrated that the record read count can be used to probabilistically identify the correct ammunition used in a particular trial, even after ammunition marked with different DNA taggants have been used in the firearm in previous trials. These capabilities are critical for civilian firearms tracing and ammunition stockpile management, where different sets of labelled ammunition could be used in the same firearm. The capacity to probabilistically relate the record rank to the ammunition used is therefore a particularly important aspect of the technology disclosed here.

(2) UniKey-Tag 2 Ammunition Fingerprinting Experiments with Series 1 Taggants (9 mm Handgun): Decoding by Fragment Length Separation.

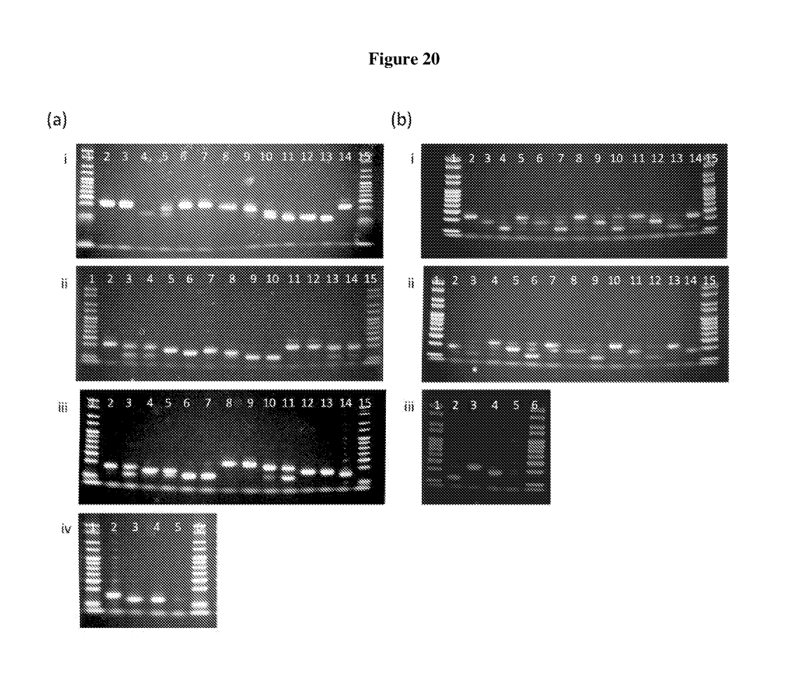

For the UniKey-Tag 2 system, samples were amplified by ATD PCR and decoded by fragment length separation gel electrophoresis. Photographs of the polyacrylamide electrophoresis gels are given in FIGS. 20-22 and the results are summarized in Tables 11 and 12. The ammunition tracing experiments were conducted over two experimental phases (Ser1_a and Ser1_b) using a 9 mm handgun. In experiment (a), two 9 mm cartridges were marked with each of the dsDNA taggants OligoTags_1-20_Ser1 given in Table 1. In the second experiment (b), ten cartridges were marked with OligoTags_4,12,20_Ser1. In Series 1(a) experiments, the five taggant groups of post-amplification length 74, 70, 64, 60, 54 bp were sometimes difficult to resolve using polyacrylamide gel electrophoresis. This was addressed in Series 1(b) experiments where three different sized taggant groups of length 74, 64, and 54 offered better band resolution.

Key results of UniKey-Tag 2 (Series 1, 9 mm) experiments are presented below.

(A) the Feasibility of the UniKey-Tag System and ATD PCR were Demonstrated in the Field

Results of the UniKey-Tag 2 ammunition tracing experiments are summarized in Table 11 and Table 12, and shown in the photographs of electrophoresis gels in FIGS. 20-22. The electrophoresis gels show clear distinct bands with no evidence of cross-fragment hybridisation in both experiment (a) and (b). This is a particularly positive result considering that the Gibbs free energy of reactions (ΔG) between the ssDNA taggants strongly favours cross-fragment hybridisation (ΔG=−44.5 and −37.9 kcal mol⁻¹) over conventional primer annealing (−32.7 kcal mol⁻¹), and is approximately four times more negative than the recommended design limit of −9.0 kcal mol⁻¹. The Gibbs reaction energy between ssDNA taggants were given in Table 2. These results demonstrate the viability of the UniKey-Tag system in the field.

### (B) Synthetic DNA is a Suitable Media for Ammunition Tracing.

The results presented in FIGS. 19-22 show that synthetic DNA remains intact on a bullet after firing, and that synthetic DNA is a suitable media to mark and trace ammunition.

(C) Taggants are Dispersed onto and Recoverable from the User, Firearm, Casing, Entry Point and Bullet after Firing—Electrophoresis Gel Results

The combined results of the UniKey-Tag 2 ammunition tracing feasibility studies (Exp Ser1_a, Ser1_b) are given in Tables 11 and 12 and summarized in FIG. 16. These results show that an unbroken chain of identification was established in almost all trials for the 9 mm handgun (n=70), linking the taggant to the user (80%), gun (100%), casing (100%), bullet (97%) and entry wound (99%), with any one of these recovery points sufficient for user identification. In total, one ammunition case and eight bullets were not recovered, and three bullet entry sites overlapped previous entry sites.

The results in Tables 11 and 12, and FIG. 20-22 show that multiple banding occurred in the cases, entry wound, and bullet samples on 43%, 33%, and 92% of occasions, respectively. Multiple banding indicates either the transfer of taggants onto other bullets when loading the magazine. As only one gun was available, it is almost certain that some transfer would have occurred. This transfer could aid forensic investigations by providing a molecular history of ammunition previously used in the firearm, and is therefore not viewed as a negative aspect of the technology.

The multiple bands in the ammunition case samples are attributed to contact transfer whilst loading the magazine. For samples collected from the bullet entry sites on the biological target material (a section of pig carcass was used to simulate human tissue), multiple banding was only observed when swab recovery techniques were used in Exp. (a). In Exp. (b) a small amount of tissue was excised from the entry wound, incubated in buffer, and introduced directly into the PCR wells. Using this second technique, 100% recovery was achieved with only the expected bands observed in each trial. These experiments showed that nucleases present in biological material did not negatively impact taggant recovery and amplification. Taggant recovery at the entry site was the ultimate goal of this study since firearms crime is invariably accompanied by a bullet hole in something or someone. Being able to identify a user, from the entry site alone, could be a very useful forensic tool.

Taggant recovery from the hand and gun was successful in 80% (n=5) and 100% (n=6) of trials respectively. Note that recovery from the hand and gun was performed at 10-shot intervals. This feasibility study proved the viability of UniKey-Tag technology to trace firearms the field. With further refinements to the recovery protocol, the rate of positive identification from the hand and gun is anticipated to improve significantly.

### (E) PVA Gel Lifts Outperform Conventional Buccal Swabs and Tape-Lift Techniques.

One important result was the successful use of PVA-based gels to recover and amplify taggants from the skin and hard surfaces on the firearm. Conventional forensic protocols use buccal swabs to recover genomic DNA from crime scenes; however, it was found that the success rate of this technique for recovering trace-level DNA taggants under controlled laboratory conditions was around only 50%. Tape-lifts were also trialed, briefly, but the additional steps required to purify DNA from the tape adhesives was deemed unsuitable for low copy number recovery.

The success of PVA-gel lifts over other conventional techniques was attributed to three main factors. Firstly, the application of a liquid gel is thought to permeate fissures and irregularities in surfaces better than tape. Secondly, the mechanical action of tearing the PVA ‘skin’ from hand or gun may improve recovery results (in a similar manner to a tape-lifts). Thirdly, the compatibility of PVA-lifts with direct PCR protocols, and the low inhibitory effect of PVA on polymerases (only Phire Hot Start II was tested), allowed the PVA ‘skin-lift’ to be added directly into the PCR wells. This bypassed the need for several additional purification steps that inevitably incur sample loss.

| | | Post | | | |
|---|---|---|---|---|---|
| | Order | amplification | | | |
| Oligo_Tag | fixed | length (bp) | Fixing agent | Sand type | Shell |
| 1 | 1 | 74 | PVA | Coarse | ***(74) |
| 1 | 1 | 74 | PVA | Coarse | ***(74) |
| 2 | 2 | 74 | TRE | Coarse | ***(74) |
| 2 | 2 | 74 | TRE | Coarse | ***(74) |
| 3 | 3 | 74 | PVA | Fine | ***(74) |
| 3 | 3 | 74 | PVA | Fine | ***(74) |
| 4 | 4 | 74 | TRE | Fine | ***(74) |
| 4 | 4 | 74 | TRE | Fine | ***(74) |
| 5 | 5 | 70 | PVA | Fine | ***(70) |
| 5 | 5 | 70 | PVA | Coarse | ***(70) |
| 6 | 9 | 70 | TRE | Coarse | ***(70), *(54) |
| 6 | 9 | 70 | TRE | Coarse | ***(70), *(54) |
| 7 | 13 | 70 | PVA | Coarse | ***(70), *(54) |
| 7 | 13 | 70 | PVA | Coarse | ***(70), *(54) |
| 8 | 17 | 70 | TRE | Coarse | ***(70), *(54) |
| 8 | 17 | 70 | TRE | Fine | ***(70), ***(54) |
| 9 | 6 | 64 | PVA | Fine | ***(64) |
| 9 | 6 | 64 | PVA | Coarse | ***(64) |
| 10 | 10 | 64 | TRE | Coarse | ***(64) |
| 10 | 10 | 64 | TRE | Coarse | ***(64) |
| 11 | 14 | 64 | PVA | Coarse | ***(64) |
| 11 | 14 | 64 | PVA | Coarse | ***(64), **(54) |
| 12 | 18 | 64 | TRE | Coarse | ***(64) |
| 12 | 18 | 64 | TRE | Fine | ***(64) |
| 13 | 7 | 60 | PVA | Fine | ***(60) |
| 13 | 7 | 60 | PVA | Fine | ***(60) |
| 14 | 11 | 60 | TRE | Fine | ***(60) |
| 14 | 11 | 60 | TRE | Fine | ***(60) |
| 15 | 15 | 60 | PVA | Fine | ***(60) |
| 15 | 15 | 60 | PVA | Fine | ***(60), *(54) |
| 16 | 19 | 60 | TRE | Fine | ***(60) |
| 16 | 19 | 60 | TRE | Fine | ***(60) |
| 17 | 8 | 54 | PVA | Fine | ***(54) |
| 17 | 8 | 54 | PVA | Fine | ***(54) |
| 18 | 12 | 54 | TRE | Fine | ***(54) |
| 18 | 12 | 54 | TRE | Fine | ***(54) |
| 19 | 16 | 54 | PVA | Fine | ***(54) |
| 19 | 16 | 54 | PVA | Fine | ***(54) |
| 20 | 20 | 54 | TRE | Fine | ***(54) |
| 20 | 20 | 54 | TRE | Fine | ***(54) |
| 1, 2, 3, 4 | | 80 only | PVA and TRE | Fine/Coarse | NA |
| 5, 9, 13, 17 | | 70, 64 ,60, 54 | PVA only | Fine/Coarse | NA |
| 6, 10, 14, 18 | | 70, 64 ,60, 54 | TRE only | Fine/Coarse | NA |
| 7 ,11, 16, 19 | | 70, 64 ,60, 54 | PVA only | Fine/Coarse | NA |
| 8, 12, 18, 20 | | 70, 64 ,60, 54 | TRE only | Fine | NA |
| | | Entry | | | |
| | Oligo_Tag | point | Bullet | Gun | Hand |
| | 1 | ***(74) | ***(74), *(54) | NA | NA |
| | 1 | ***(74) | ***(74), *(54) | NA | NA |
| | 2 | ***(74), *(54) | **(74), *(64), *(54) | NA | NA |
| | 2 | ***(74), ***(54) | **(74), *(64), *(54) | NA | NA |
| | 3 | ***(74), *(70), *(54) | **(74), *(64), **(54) | NA | NA |
| | 3 | ***(74), *(70), *(54) | **(74), *(64), **(54) | NA | NA |
| | 4 | ***(74) | **(74), *(64), **(54) | NA | NA |
| | 4 | ***(74), *(54) | **(74), *(64), **(54) | NA | NA |
| | 5 | *(70) | ***(70), *(54) | NA | NA |
| | 5 | *(70), **(60) | ***(70), *(54) | NA | NA |
| | 6 | **(70), *(64), **(60) | ***(70), *(54) | NA | NA |
| | 6 | **(70), *(64), **(60) | ***(70), *(64), *(54) | NA | NA |
| | 7 | **(70), **(54) | ***(70) | NA | NA |
| | 7 | *(70), ***(54) | **(70), *(64), **(54) | NA | NA |
| | 8 | *(70), ***(54) | NA mixed | NA | NA |
| | 8 | NR | NA mixed | NA | NA |
| | 9 | *(70), *(64), ***(60) | *(74), **(70), *(64), **(54) | NA | NA |
| | 9 | ***(64), ***(60) | *(74), **(70), *(64), **(54) | NA | NA |
| | 10 | ***(64), *(60) | *(74), **(70), *(64), **(54) | NA | NA |
| | 10 | ***(64), *(60) | *(74), **(70), *(64), **(54) | NA | NA |
| | 11 | **(64), **(54) | ***(64), *(60), **(54) | NA | NA |
| | 11 | **(64) | ***(64), *(60), **(54) | NA | NA |
| | 12 | ***(64) | NA mixed | NA | NA |
| | 12 | NR | NA mixed | NA | NA |
| | 13 | **(60) | *(70), ***(64), **(60) | NA | NA |
| | 13 | ***(60) | ***(70), *(64), **(60) | NA | NA |
| | 14 | *(74), ***(60), *(54) | **(74), *(64), **(60) | NA | NA |
| | 14 | *(74), ***(60), *(54) | *(74), *(64), **(60) | NA | NA |
| | 15 | ***(60), *(54) | **(74), *(60) | NA | NA |
| | 15 | ***(60), *(54) | **(74), **(60) | NA | NA |
| | 16 | ***(60) | NA mixed | NA | NA |
| | 16 | ***(60) | NA mixed | NA | NA |
| | 17 | ***(54) | ***(74), *(64), **(54) | NA | NA |
| | 17 | ***(54) | ***(74), *(64), **(54) | NA | NA |
| | 18 | *(74), ***(54) | ***(54), *(74) | NA | NA |
| | 18 | *(74), ***(54) | ***(54), *(74) | NA | NA |
| | 19 | ***(54) | **(74), *(64), **(54) | NA | NA |
| | 19 | ***(54) | **(74), *(64), **(54) | NA | NA |
| | 20 | ***(54) | NA mixed | NA | NA |
| | 20 | NR | NA mixed | NA | NA |
| | 1, 2, 3, 4 | NA | NA | ***(74) | ***(74) |
| | 5, 9, 13, 17 | NA | NA | ***(54, 60, 64, 70, 74) | ***(54, 60, 64, 70, 74) |
| | 6, 10, 14, 18 | NA | NA | ***(54, 60, 64, 70, 74) | ***(54, 60, 64, 70, 74) |
| | 7 ,11, 16, 19 | NA | NA | ***(54, 60, 64, 70, 74) | ***(54, 60, 64, 70, 74) |
| | 8, 12, 18, 20 | NA | NA | ***(54, 60, 64, 70, 74) | [symbol] |

[symbol]

indicates data missing or illegible when filed

| | | Post | | | | | | | |
|---|---|---|---|---|---|---|---|---|---|
| | Order | amplification | Fixing | Sand | | Entry | | | |
| Oligo_tag | fixed | length (bp) | agent | type | Casing | point | Bullet | Gun | Hand |
| 4 | 1 | 74 | PVA | Fine | ***(74) | ***(74) | ***(74), **(54) | NA | NA |
| 12 | 2 | 64 | PVA | Fine | **(64) | ***(64) | **(74), ***(64), *(54) | NA | NA |
| 20 | 3 | 54 | PVA | Fine | *(74), *(64), ***(54) | ***(54) | *(74), ***(54) | NA | NA |
| 4 | 4 | 74 | PVA | Fine | ***(74), *(64), *(54) | ***(74) | ***(74), *(54) | NA | NA |
| 12 | 5 | 64 | PVA | Fine | *(74), **(64), *(54) | ***(64) | ***(74), **(64), *(54) | NA | NA |
| 20 | 6 | 54 | PVA | Fine | *(74), **(64), ***(54) | ***(54) | **(74), ***(54) | NA | NA |
| 4 | 7 | 74 | PVA | Fine | ***(74), *(64), *(54) | ***(74) | ***(74), *(54) | NA | NA |
| 12 | 8 | 64 | PVA | Fine | *(74), ***(64), *(54) | ***(64) | ***(74), *(64), **(54) | NA | NA |
| 20 | 9 | 54 | PVA | Fine | **(74), *(64), ***(54) | ***(54) | *(74), ***(54) | NA | NA |
| 4 | 10 | 74 | PVA | Fine | ***(74), *(64), *(54) | ***(74) | ***(74), **(54) | NA | NA |
| 12 | 11 | 64 | PVA | Fine | *(74), ***(64), *(54) | ***(64) | **(74), **(64), *(54) | NA | NA |
| 20 | 12 | 54 | PVA | Fine | *(74), *(64), **(54) | ***(54) | *(74), *(64), ***(54) | NA | NA |
| 4 | 13 | 74 | PVA | Fine | ***(74), *(54) | ***(74) | ***(74) | NA | NA |
| 12 | 14 | 64 | PVA | Fine | **(64) | ***(64) | *(74), ***(64), ***(54) | NA | NA |
| 20 | 15 | 54 | PVA | Fine | *(74), *(64), **(54) | ***(54) | ***(64), ***(54) | NA | NA |
| 4 | 16 | 74 | PVA | Fine | ***(74), *(64), *(54) | ***(74) | *(74), **(64), ***(54) | NA | NA |
| 12 | 17 | 64 | PVA | Fine | *(74), ***(64), *(54) | ***(64) | ***(64), *(54) | NA | NA |
| 20 | 18 | 54 | PVA | Fine | *(74), *(64), ***(54) | ***(54) | **(64), ***(54) | NA | NA |
| 4 | 19 | 74 | PVA | Fine | ***(74), **(64), *(54) | ***(74) | *(74), **(64), **(54) | NA | NA |
| 12 | 20 | 64 | PVA | Fine | *(74), ***(64), *(54) | ***(64) | (74), **(64), ***(54) | NA | NA |
| 20 | 21 | 54 | PVA | Fine | *(74), *(64), ***(54) | ***(54) | *(74), *(64), ***(54) | NA | NA |
| 4 | 22 | 74 | PVA | Fine | ***(74), *(64), *(54) | ***(74) | ***(74), *(64), *(54) | NA | NA |
| 12 | 23 | 64 | PVA | Fine | *(74), **(64), *(54) | ***(64) | **(64), *(54) | NA | NA |
| 20 | 24 | 54 | PVA | Fine | *(74), *(64), **(54) | ***(54) | *(64), ***(54) | NA | NA |
| 4 | 25 | 74 | PVA | Fine | ***(74) | ***(74) | *(64), ***(54) | NA | NA |
| 12 | 26 | 64 | PVA | Fine | **(64) | ***(64) | *(64), **(54) | NA | NA |
| 20 | 27 | 54 | PVA | Fine | *(74), ***(54) | ***(54) | ***(54) | NA | NA |
| 4 | 28 | 74 | PVA | Fine | ***(74) | ***(74) | ***(74) | NA | NA |
| 12 | 29 | 64 | PVA | Fine | **(64) | ***(64) | ***(64), **(54) | NA | NA |
| 20 | 30 | 54 | PVA | Fine | **(54) | ***(54) | **(54) | NA | NA |
| 4, 12, 20 | | | | | NA | NA | NA | **(74), | No **(74), |
| | | | | | | | | **(64), **(54) | **(64), No(54) |

### Example 7—Molecular Taggant Technology for Tracing Counterfeit Pharmaceuticals

The UniKey taggants and ATD PCR with LNA primers are suitable for deep layering applications such as supply chain tracing in the pharmaceuticals industry. The capacity to screen billions of taggants simultaneously allows tagged product precursors to be mixed and decoded from the final product in one reaction. This deep layering capability is unique to the presently claimed invention, and has been illustrated in FIG. 1. The taggants may contain product information such as expiry date, manufacturer, manufacturing facility, batch number, etc. such that the subset of taggants contains this information for all precursors. Alternatively, the taggents may encode a unique serial number that is used to look up product information on a centralized database. Conceptually, the entire industry could use one set of universal primers (i.e. the same library) so that any mixture of pharmaceutical products could be decoded in one reaction. For security reasons, however, it may be desirable to use multiple sets of universal primers.

The UniKey-Tag 1 system was additionally tested for the purpose of labelling pharmaceuticals. In these experiments, Series 1 Ham(8,4,4) encoded taggants (given in Table 4) were used to label five commonly counterfeited drugs: Riamet (malaria anti-parasitic), Isoniazid (tuberculosis antibiotic), Amoxycilin and clavulanic acid (broad-spectrum antibiotic) and Cialis (erectile dysfunction). Different dsDNA taggants were mixed into the drugs at a concentration of 0.001, 0.01, 0.1, 1, and 10 ng g⁻¹ of tablet (see Table 13). Multiple taggants were used to label these drugs to simulate multiple precursor labelling as shown in FIG. 1. The taggants were recovered, sequenced and decoded using the direct PCR protocols described previously for the ammunition UniKey-Tag 1 tracing experiments.

Results of the pharmaceuticals labelling experiments are given in Table 13. For all drug types, and across almost all concentrations tested, the DNA taggants were successfully recovered and decoded.

TABLE 13
Results of the pharmaceutical labeling experiments

| | | Pharmaceuticals tested | | | | |
|---|---|---|---|---|---|---|
| Concentration | Concentration | | | | Amoxycilin | |
| DNA tag | DNA tag | | | | and C- | |
| ng g⁻¹ tablet | nmol g⁻¹ tablet | Riamet | Isoniazid | Rifampicin | acid | Cialis |
| 10 | 0.000175439 | 3/3 | 2/2 | 2/2 | 3/3 | 10/10 |
| 1 | 0.000017543 | 3/3 | 2/2 | 2/2 | 3/3 | 9/10 |
| 0.1 | 0.000001754 | 3/3 | 2/2 | 2/2 | 3/3 | 10/10 |
| 0.01 | 0.000000175 | 3/3 | 2/2 | 2/2 | 3/3 | 9/10 |
| 0.001 | 0.000000017 | 3/3 | 2/2 | 2/2 | NA | 10/10 |

| | | | | | |
|---|---|---|---|---|---|
| Labels, OligoTag (Series 2): | 1, 2, 3 | 4, 5 | 6,7 | 8, 9, 10 | 1-10 |

### Example 8—Molecular Taggant Technology for DNA-Based Archival Storage

The capacity of existing data storage media is lagging behind the rate at which new data is generated. The use of DNA for archival data storage is attractive because it is information dense (10⁹ GB mm⁻³, eight orders of magnitude more dense than tape), has a long half-life (approx. 500 years for 100 bp fragments under most conditions) and is synthesised and sequenced using commercially mature technologies. Conventional optical, magnetic and flash technologies, on the other hand, have a lifespan of 5-30 years and require constant renewal. Accordingly, DNA as a long-term storage media has gained significant interest in view of the limitations of conventional data storage technologies.

DNA-based storage also has the benefit of eternal relevance: as long as there is DNA-based life, there will be strong reasons to read and manipulate DNA. The write process for DNA storage maps digital data into DNA nucleotide sequences (a nucleotide is the basic building block of DNA), synthesizes (manufactures) the corresponding DNA molecules, and stores them away. Reading the data involves sequencing the DNA molecules and decoding the information back to the original digital data. Both synthesis and sequencing are standard practice in biotechnology, from research to diagnostics and therapeutics.

Progress in DNA storage has been rapid since 1999, when DNA-based storage was encoding and recovering a 23 character message⁹. The volume of data that can be synthesized today is limited mostly by the cost of synthesis and sequencing, but growth in the biotechnology industry portends orders of magnitude cost reductions and efficiency improvements.

The paper by Bornholt et al. (2016; A DNA-Based Archival Storage System. APLOS) presents an architecture for a DNA-backed archival storage system, modeled as a key-value store. The paper highlights several challenges that need to be overcome. First, DNA synthesis and sequencing is far from perfect, with error rates on the order of 1%. Sequences can also degrade while stored, further compromising data integrity. A key aspect of DNA storage is to devise appropriate encoding schemes that can tolerate errors by adding redundancy. Existing approaches have focused on redundancy but have ignored density implications. The work proposes a new encoding scheme that offers controllable redundancy, enabling different types of data (e.g., text and images) to have different levels of reliability and density. A second problem identified is that randomly accessing data in DNA-based storage is problematic, resulting in overall read latency that is much longer than write latency. Additionally, as the fragments of DNA used to encode files are stored in a solution the coordinate systems used to access data in conventional media cannot be used. Existing work has provided only large-block access: to read even a single byte from storage the entire DNA pool must be sequenced and decoded.

### Annealing Temperature Discrimination PCR for Random Access Archival Data Recovery

As previously described, annealing temperature discrimination PCR (ATD PCR) allows a subset of taggants to be simultaneously selected and amplified from a pool of taggants without the occurrence of cross-fragment hybridisation. This capability translates perfectly to random access or small scale block access data recovery in DNA-based archival storage systems. For example, FIG. 2 shows three image files that are encoded by a specific library of fragments (Wₐ, W_{b}, and W_{c}). Each library is defined by a specific set of forward and reverse primer sites that are universal to the file (e.g., UPFb, UPRb). The files are archived as a mixed pool of DNA fragments (P) comprising data, in DNA form, for all three pictures. ATD PCR allows random access of a particular picture file in one reaction, without cross fragment hybridisation. ATD PCR also permits much greater encoding flexibility by reducing the incidence of variable region heterodimer formation. This is a particular problem when two different DNA fragments contain the same symbol subsequence in the codeword as described previously (see also FIG. 5). The ATD PCR amplification products are then sent for sequencing and the picture is decoded from the resulting sequence. Files may also be divided into smaller library sets to allow for higher resolution access capability; for example, to access a particular part of a file. Note that fragments (τ) within each library would require an index sequence inside the variable region so that files can be reconstructed. In reality each picture file may be encoded with thousands of fragments and thousands of picture files may be stored together in a mixture.

Although this system does not have rewriting capabilities, a specific part of a file could be changed by simply adding updated sets of fragments to the pool with an additional code in the variable region identifying the fragment as the most up to date version. In any case, rewrite-ability is not viewed as a critical element of archival data storage.

### REFERENCES

1. Bornholt, J. et al. A DNA-based archival storage system. ASPLOS '16—Proc. Twenty-First Int. Conf. Archit. Support Program. Lang. Oper. Syst. 637-649 (2016). doi:10.1145/2872362.2872397.
2. Bystrykh, L. V. Generalized DNA barcode design based on Hamming codes. PLoS One 7, 1-8 (2012).
3. Church, G. M., Gao, Y. & Kosuri, S. Next-Generation Digital Information Storage in DNA. Science (80-.). 399, 533-534 (2013).
4. Goldman, N. et al. Toward practical high-capacity low-maintenance storage of digital information in synthesised DNA. Nature 494, 77-80 (2013).
5. Hamming, R. W. Error detecting and error correcting codes. Bell Syst. Tech. J. 29, (1950).
6. Levenshtein, V. I. Binary codes capable of correcting deletion, insertions and reversals. Sov. Physics-Doklady 10, 707-710 (1966).
7. Reed, I. S. & Solomon, G. Polynomial codes over certain finite fields. J. Soc. Ind. Appl. Math. 8.2 300-304 (1960).
8. Tabatabaei Yazdi, S. M. H., Yuan, Y., Ma, J., Zhao, H. & Milenkovic, O. A Rewritable, Random-Access DNA-Based Storage System. Nature 5, 14138 (2015).
9. Clelland, C. T., Risca, V. & Bancroft, C. Hiding messages in DNA microdots. Nature 399, 533-534 (1999).

1-13. (canceled)

14. A method of tracing a product to its origin, the method comprising:
(a) providing a product to which at least one nucleic acid sequence has been incorporated, wherein the at least one nucleic acid sequence is flanked by a first primer site and a second primer site;
(b) optionally recovering the at least one nucleic acid sequence from the product;
(c) amplifying the at least one nucleic acid sequence by high fidelity amplification comprising thermocycling using a first primer complementary to the first primer site and a second primer complementary to the second primer site, wherein the first and second primers each comprise at least one locked nucleic acid (LNA), wherein the thermocycling comprises a melting phase, an annealing phase and an extension phase, and wherein an elevated temperature is used during the annealing phase of the thermocycling such that, during the annealing phase, there is substantially no annealing of nucleic acid sequences other than of the first and second primers to the first and second primer sites, respectively; and
(d) identifying the at least one nucleic acid sequence amplified in step (c);
wherein the sequence and/or length of the at least one nucleic acid sequence identified in step (d) is indicative of the origin of the product.

15-41. (canceled)

42. A method of tracing a product to its origin, the method comprising:
(a) providing a product to which two or more different nucleic acid sequences have been incorporated, wherein each of the two or more nucleic acid sequences comprises a string of subsequences that encodes non-biological information, and wherein each of the two or more nucleic acid sequences are flanked by a common first primer site and a common second primer site;
(b) optionally recovering the two or more nucleic acid sequences from the product;
(c) amplifying the two or more nucleic acid sequences by high fidelity amplification in a single thermocycling reaction using a first primer complementary to the first primer site and a second primer complementary to the second primer site, wherein the first and second primers each comprise at least one locked nucleic acid (LNA), wherein the thermocycling comprises a melting phase, an annealing phase and an extension phase, and wherein an elevated temperature is used during the annealing phase of the thermocycling such that, during the annealing phase, there is substantially no annealing of nucleic acid sequences other than of the first and second primers to the first and second primer sites, respectively; and
(d) identifying the two or more nucleic acid sequences amplified in step (c);
wherein the sequence and/or length of the two or more nucleic acid sequences identified in step (d) is indicative of the origin of the product.

43. The method of claim 42, wherein the product is selected from the group consisting of a firearm, ammunition, projectile, and firearm residue.

44. The method of claim 42, wherein the product is a pharmaceutical product or a precursor thereof.

45. The method of claim 42, wherein step (d) comprises identifying the amplified at least one nucleic acid sequence by sequencing.

46. The method of claim 42, wherein each of the two or more nucleic acid sequences has a different length.

47. The method of claim 42, wherein step (d) comprises identifying the amplified at least one nucleic acid sequence by size separation.

48. The method of claim 42, wherein the first and second primers each comprises 8 to 30 nucleotides.

49. The method of claim 42, wherein the first and second primers each comprises 15 to 25 nucleotides.

50. The method of claim 42, wherein the temperature used during the annealing phase is at least 5° C. higher than the temperature at which nucleic acid sequences other than the first and second primers would anneal.

51. The method of claim 50, wherein the temperature used during the annealing phase is at least 10° C. higher than the temperature at which nucleic acid sequences other than the first and second primers would anneal.

52. The method of claim 42, wherein the temperature used during the annealing phase is about 50° C. to 72° C.

53. The method of claim 52, wherein the temperature used during the annealing phase is about 67° C. to 72° C.

54. The method of claim 42, wherein each of the first and second primers comprises 1 to 8 LNA.

55. The method of claim 42, wherein the first and/or second primer comprise a nucleic acid sequence selected from SEQ ID NOs: 61 to 68.

56. A method for high fidelity amplification of two or more target nucleic acid sequences in a mixture thereof, wherein each of the two or more target nucleic acid sequences are flanked by a first primer site and a second primer site, wherein the amplification comprises thermocycling comprising a melting phase, an annealing phase and an extension phase, the method comprising using a first primer complementary to each of the first primer sites and a second primer complementary to each of the second primer sites, wherein each of the first and second primers comprise at least one locked nucleic acid (LNA) and wherein an elevated temperature is used during the annealing phase of the thermocyling such that, during the annealing phase, there is substantially no annealing of nucleic acid sequences other than of the first and second primers to the first and second primer sites, respectively,
wherein one or more or all of the following apply,
i) the two or more target nucleic acid sequences are amplified in a single thermocycling reaction;
ii) the two or more target nucleic acid sequences encode non-biological information; or
iii) each of the two or more target nucleic acids are flanked by a common first primer site and a common second primer site.


SEQUENCE LISTING (68 sequences)

SEQ ID NO: 1 - DNA, 80 bp, Artificial Sequence
  Synthetic nucleic acid tag
atcatctgactttcgtatctagcaggatgctgtgcaaggagaagcagctacgtgtcaccatgagaagttcatacacatat

SEQ ID NO: 2 - DNA, 80 bp, Artificial Sequence
  Synthetic nucleic acid tag
atatgtgtatgaacttctcatggtgacacgtagctgcttctccttgcacagcatcctgctagatacgaaagtcagatgat

SEQ ID NO: 3 - DNA, 80 bp, Artificial Sequence
  Synthetic nucleic acid tag
atcatctgactttcgtatctagcacctatcgactcgtgtacgtggattcgttctacaccatgagaagttcatacacatat

SEQ ID NO: 4 - DNA, 80 bp, Artificial Sequence
  Synthetic nucleic acid tag
atatgtgtatgaacttctcatggtgtagaacgaatccacgtacacgagtcgataggtgctagatacgaaagtcagatgat

SEQ ID NO: 5 - DNA, 80 bp, Artificial Sequence
  Synthetic nucleic acid tag
atcatctgactttcgtatctagcacagtcgatccttcgtacctgcttcgatggcaatccatgagaagttcatacacatat

SEQ ID NO: 6 - DNA, 80 bp, Artificial Sequence
  Synthetic nucleic acid tag
atatgtgtatgaacttctcatggattgccatcgaagcaggtacgaaggatcgactgtgctagatacgaaagtcagatgat

SEQ ID NO: 7 - DNA, 80 bp, Artificial Sequence
  Synthetic nucleic acid tag
atcatctgactttcgtatctagcaggttgctgagtacgtgaacggaccacttgcactccatgagaagttcatacacatat

SEQ ID NO: 8 - DNA, 80 bp, Artificial Sequence
  Synthetic nucleic acid tag
atatgtgtatgaacttctcatggagtgcaagtggtccgttcacgtactcagcaacctgctagatacgaaagtcagatgat

SEQ ID NO: 9 - DNA, 76 bp, Artificial Sequence
  Synthetic nucleic acid tag
atcatctgactttcgtatctagcaagctctcgtacagtgacgcactacactcaccatgagaagttcatacacatat

SEQ ID NO: 10 - DNA, 76 bp, Artificial Sequence
  Synthetic nucleic acid tag
atatgtgtatgaacttctcatggtgagtgtagtgcgtcactgtacgagagcttgctagatacgaaagtcagatgat

SEQ ID NO: 11 - DNA, 76 bp, Artificial Sequence
  Synthetic nucleic acid tag
atcatctgactttcgtatctagctcctagacgactgctaccatccaagcgactccatgagaagttcatacacatat

SEQ ID NO: 12 - DNA, 76 bp, Artificial Sequence
  Synthetic nucleic acid tag
atatgtgtatgaacttctcatggagtcgcttggatggtagcagtcgtctaggagctagatacgaaagtcagatgat

SEQ ID NO: 13 - DNA, 76 bp, Artificial Sequence
  Synthetic nucleic acid tag
atcatctgactttcgtatctagccttcagagagcactcttcatgcaggttgcaccatgagaagttcatacacatat

SEQ ID NO: 14 - DNA, 76 bp, Artificial Sequence
  Synthetic nucleic acid tag
atatgtgtatgaacttctcatggtgcaacctgcatgaagagtgctctctgaaggctagatacgaaagtcagatgat

SEQ ID NO: 15 - DNA, 76 bp, Artificial Sequence
  Synthetic nucleic acid tag
atcatctgactttcgtatctagcatctcctgattgcactgctttgcgttgcgaccatgagaagttcatacacatat

SEQ ID NO: 16 - DNA, 76 bp, Artificial Sequence
  Synthetic nucleic acid tag
atatgtgtatgaacttctcatggtcgcaacgcaaagcagtgcaatcaggagatgctagatacgaaagtcagatgat

SEQ ID NO: 17 - DNA, 70 bp, Artificial Sequence
  Synthetic
atcatctgactttcgtatctagctgacctgaacgtggacctcagactccatgagaagttcatacacatat

SEQ ID NO: 18 - DNA, 70 bp, Artificial Sequence
  Synthetic nucleic acid tag
atatgtgtatgaacttctcatggagtctgaggtccacgttcaggtcagctagatacgaaagtcagatgat

SEQ ID NO: 19 - DNA, 70 bp, Artificial Sequence
  Synthetic nucleic acid tag
atcatctgactttcgtatctagcacatccctaccaacgcactaccagccatgagaagttcatacacatat

SEQ ID NO: 20 - DNA, 70 bp, Artificial Sequence
  Synthetic nucleic acid tag
atatgtgtatgaacttctcatggctggtagtgcgttggtagggatgtgctagatacgaaagtcagatgat

SEQ ID NO: 21 - DNA, 70 bp, Artificial Sequence
  Synthetic
atcatctgactttcgtatctagcactcggttagctgcaaggaccactccatgagaagttcatacacatat

SEQ ID NO: 22 - DNA, 70 bp, Artificial Sequence
  Synthetic nucleic acid tag
atatgtgtatgaacttctcatggagtggtccttgcagctaaccgagtgctagatacgaaagtcagatgat

SEQ ID NO: 23 - DNA, 70 bp, Artificial Sequence
  Synthetic nucleic acid tag
atcatctgactttcgtatctagcgttcgttgcaggtctacacgatcaccatgagaagttcatacacatat

SEQ ID NO: 24 - DNA, 70 bp, Artificial Sequence
  Synthetic nucleic acid tag
atatgtgtatgaacttctcatggtgatcgtgtagacctgcaacgaacgctagatacgaaagtcagatgat

SEQ ID NO: 25 - DNA, 66 bp, Artificial Sequence
  Synthetic nucleic acid tag
atcatctgactttcgtatctagcttcctcagcagagtcggagtccatgagaagttcatacacatat

SEQ ID NO: 26 - DNA, 66 bp, Artificial Sequence
  Synthetic
atatgtgtatgaacttctcatggactccgactctgctgaggaagctagatacgaaagtcagatgat

SEQ ID NO: 27 - DNA, 66 bp, Artificial Sequence
  Synthetic nucleic acid tag
atcatctgactttcgtatctagctgagccactagcctctgaacccatgagaagttcatacacatat

SEQ ID NO: 28 - DNA, 66 bp, Artificial Sequence
  Synthetic nucleic acid tag
atatgtgtatgaacttctcatgggttcagaggctagtggctcagctagatacgaaagtcagatgat

SEQ ID NO: 29 - DNA, 66 bp, Artificial Sequence
  Synthetic nucleic acid tag
atcatctgactttcgtatctagcagcgttcactcgaacctacaccatgagaagttcatacacatat

SEQ ID NO: 30 - DNA, 66 bp, Artificial Sequence
  Synthetic nucleic acid tag
atatgtgtatgaacttctcatggtgtaggttcgagtgaacgctgctagatacgaaagtcagatgat

SEQ ID NO: 31 - DNA, 66 bp, Artificial Sequence
  Synthetic nucleic acid tag
atcatctgactttcgtatctagcaccgtgcgagtgtagcaagtccatgagaagttcatacacatat

SEQ ID NO: 32 - DNA, 66 bp, Artificial Sequence
  Synthetic nucleic acid tag
atatgtgtatgaacttctcatggacttgctacactcgcacggtgctagatacgaaagtcagatgat

SEQ ID NO: 33 - DNA, 60 bp, Artificial Sequence
  Synthetic nucleic acid tag
atcatctgactttcgtatctagcaggcagtcgtgcttccatgagaagttcatacacatat

SEQ ID NO: 34 - DNA, 60 bp, Artificial Sequence
  Synthetic nucleic acid tag
atatgtgtatgaacttctcatggaagcacgactgcctgctagatacgaaagtcagatgat

SEQ ID NO: 35 - DNA, 60 bp, Artificial Sequence
  Synthetic nucleic acid tag
atcatctgactttcgtatctagctggacctcgatgctccatgagaagttcatacacatat

SEQ ID NO: 36 - DNA, 60 bp, Artificial Sequence
  Synthetic nucleic acid tag
atatgtgtatgaacttctcatggagcatcgaggtccagctagatacgaaagtcagatgat

SEQ ID NO: 37 - DNA, 60 bp, Artificial Sequence
  Synthetic nucleic acid tag
atcatctgactttcgtatctagcgagtagcaccctgaccatgagaagttcatacacatat

SEQ ID NO: 38 - DNA, 60 bp, Artificial Sequence
  Synthetic nucleic acid tag
atatgtgtatgaacttctcatggtcagggtgctactcgctagatacgaaagtcagatgat

SEQ ID NO: 39 - DNA, 60 bp, Artificial Sequence
  Synthetic nucleic acid tag
atcatctgactttcgtatctagccacctgcttccagaccatgagaagttcatacacatat

SEQ ID NO: 40 - DNA, 60 bp, Artificial Sequence
  Synthetic nucleic acid tag
atatgtgtatgaacttctcatggtctggaagcaggtggctagatacgaaagtcagatgat

SEQ ID NO: 41 - DNA, 92 bp, Artificial Sequence
  Synthetic nucleic acid tag
tttctgttggtgctgatattgcctaatcaacaaggtcttccttcctaattaaccgagccttattccttccgaagatagagcgacaggcaagt

SEQ ID NO: 42 - DNA, 92 bp, Artificial Sequence
  Synthetic nucleic acid tag
acttgcctgtcgctctatcttcggaaggaataaggctcggttaattaggaaggaagaccttgttgattaggcaatatcagcaccaacagaaa

SEQ ID NO: 43 - DNA, 92 bp, Artificial Sequence
  Synthetic nucleic acid tag
tttctgttggtgctgatattgcctatagtaacgatgtcaactggctatgcacgtgttccagacgattgacgaagatagagcgacaggcaagt

SEQ ID NO: 44 - DNA, 92 bp, Artificial Sequence
  Synthetic nucleic acid tag
acttgcctgtcgctctatcttcgtcaatcgtctggaacacgtgcatagccagttgacatcgttactataggcaatatcagcaccaacagaaa

SEQ ID NO: 45 - DNA, 92 bp, Artificial Sequence
  Synthetic nucleic acid tag
tttctgttggtgctgatattgcatatacacaactggctcatcgtgaaccttgagtgagacctgcgtacatgaagatagagcgacaggcaagt

SEQ ID NO: 46 - DNA, 92 bp, Artificial Sequence
  Synthetic nucleic acid tag
acttgcctgtcgctctatcttcatgtacgcaggtctcactcaaggttcacgatgagccagttgtgtatatgcaatatcagcaccaacagaaa

SEQ ID NO: 47 - DNA, 92 bp, Artificial Sequence
  Synthetic nucleic acid tag
tttctgttggtgctgatattgcatctcggagtatatgtattagttgaagaggagtattatggctgtgatggaagatagagcgacaggcaagt

SEQ ID NO: 48 - DNA, 92 bp, Artificial Sequence
  Synthetic nucleic acid tag
acttgcctgtcgctctatcttccatcacagccataatactcctcttcaactaatacatatactccgagatgcaatatcagcaccaacagaaa

SEQ ID NO: 49 - DNA, 92 bp, Artificial Sequence
  Synthetic nucleic acid tag
tttctgttggtgctgatattgccctcttctatcggtcagtctcaagcaaggtctagaagaggcgctatgagaagatagagcgacaggcaagt

SEQ ID NO: 50 - DNA, 92 bp, Artificial Sequence
  Synthetic nucleic acid tag
acttgcctgtcgctctatcttctcatagcgcctcttctagaccttgcttgagactgaccgatagaagagggcaatatcagcaccaacagaaa

SEQ ID NO: 51 - DNA, 92 bp, Artificial Sequence
  Synthetic nucleic acid tag
tttctgttggtgctgatattgcttaattggccacaatgcgtacatgcctcttcttcttctccagttgattgaagatagagcgacaggcaagt

SEQ ID NO: 52 - DNA, 92 bp, Artificial Sequence
  Synthetic nucleic acid tag
acttgcctgtcgctctatcttcaatcaactggagaagaagaagaggcatgtacgcattgtggccaattaagcaatatcagcaccaacagaaa

SEQ ID NO: 53 - DNA, 92 bp, Artificial Sequence
  Synthetic nucleic acid tag
tttctgttggtgctgatattgccttagagcataataccctacgacaactaatcagtaatgtttggctaacgaagatagagcgacaggcaagt

SEQ ID NO: 54 - DNA, 92 bp, Artificial Sequence
  Synthetic nucleic acid tag
acttgcctgtcgctctatcttcgttagccaaacattactgattagttgtcgtagggtattatgctctaaggcaatatcagcaccaacagaaa

SEQ ID NO: 55 - DNA, 92 bp, Artificial Sequence
  Synthetic nucleic acid tag
tttctgttggtgctgatattgctatatgtgtacacatagattagttgtacgcataacaccgtccgtaacagaagatagagcgacaggcaagt

SEQ ID NO: 56 - DNA, 92 bp, Artificial Sequence
  Synthetic nucleic acid tag
acttgcctgtcgctctatcttctgttacggacggtgttatgcgtacaactaatctatgtgtacacatatagcaatatcagcaccaacagaaa

SEQ ID NO: 57 - DNA, 92 bp, Artificial Sequence
  Synthetic nucleic acid tag
tttctgttggtgctgatattgcagctagtcatatacactgttgtaaccacaatgtaatcaacatgacgctgaagatagagcgacaggcaagt

SEQ ID NO: 58 - DNA, 92 bp, Artificial Sequence
  Synthetic nucleic acid tag
acttgcctgtcgctctatcttcagcgtcatgttgattacattgtggttacaacagtgtatatgactagctgcaatatcagcaccaacagaaa

SEQ ID NO: 59 - DNA, 92 bp, Artificial Sequence
  Synthetic nucleic acid tag
tttctgttggtgctgatattgctccttcctggagaagagctctaaggctaactgaggtctcaggtgttacgaagatagagcgacaggcaagt

SEQ ID NO: 60 - DNA, 92 bp, Artificial Sequence
  Synthetic nucleic acid tag
acttgcctgtcgctctatcttcgtaacacctgagacctcagttagccttagagctcttctccaggaaggagcaatatcagcaccaacagaaa

SEQ ID NO: 61 - DNA, 20 bp, Artificial Sequence
  Synthetic primer
atctgactttcgtatctagc

SEQ ID NO: 62 - DNA, 20 bp, Artificial Sequence
  Synthetic primer
tgtgtatgaacttctcatgg

SEQ ID NO: 63 - DNA, 20 bp, Artificial Sequence
  Synthetic primer
  LNA (4)..(4): Locked Nucleic Acid
  LNA (6)..(6): Locked Nucleic Acid
  LNA (10)..(10): Locked Nucleic Acid
  LNA (12)..(12): Locked Nucleic Acid
  LNA (13)..(13): Locked Nucleic Acid
  LNA (16)..(16): Locked Nucleic Acid
  LNA (17)..(17): Locked Nucleic Acid
atctgactttcgtatctagc

SEQ ID NO: 64 - DNA, 20 bp, Artificial Sequence
  Synthetic primer
  LNA (5)..(5): Locked nucleic acid
  LNA (9)..(9): Locked nucleic acid
  LNA (12)..(12): Locked nucleic acid
  LNA (15)..(15): Locked nucleic acid
  LNA (17)..(17): Locked nucleic acid
  LNA (19)..(19): Locked nucleic acid
tgtgtatgaacttctcatgg

SEQ ID NO: 65 - DNA, 22 bp, Artificial Sequence
  Synthetic primer
tttctgttggtgctgatattgc

SEQ ID NO: 66 - DNA, 22 bp, Artificial Sequence
  Synthetic primer
acttgcctgtcgctctatcttc

SEQ ID NO: 67 - DNA, 22 bp, Artificial Sequence
  Synthetic primer
  LNA (5)..(5): Locked nucleic acid
  LNA (8)..(8): Locked nucleic acid
  LNA (9)..(9): Locked nucleic acid
tttctgttggtgctgatattgc

SEQ ID NO: 68 - DNA, 22 bp, Artificial Sequence
  Synthetic primer
  LNA (6)..(6): Locked nucleic acid
  LNA (10)..(10): Locked nucleic acid
  LNA (11)..(11): Locked nucleic acid
acttgcctgtcgctctatcttc